(19) United States
(12) Patent Application Publication
(10) Pub. No.: US 2022/0380413 A1
(43) Pub. Date: Dec. 1, 2022

(54) Adeno-Associated Viral Vectors for Crossing the Human Blood Brain Barrier

(71) Applicant: The Board of Trustees of the Leland Stanford Junior University, Stanford, CA (US)

(72) Inventors: Mark A. Kay, Los Altos, CA (US); Ren Song, Redwood City, CA (US)

(21) Appl. No.: 17/638,509

(22) PCT Filed: Aug. 26, 2020
(86) PCT No.: PCT/US2020/047917

§ 371 Date: Feb. 25, 2022

Related U.S. Application Data
(60) Provisional application No. 62/893,723, filed on Aug. 29, 2019.

(51) Int. Cl. C07K 14/075; C12N 15/113

(52) U.S. Cl. CPC C07K 14/075; C12N 15/113; C12N 2750/14122; C12N 2750/14143

(57) ABSTRACT

The present disclosure provides variant adeno-associated virus (AAV) capsid polypeptides that provide an AAV particle with the ability to traverse the human blood brain barrier (BBB) and transduce cells of the CNS. In some embodiment, a subject variant AAV capsid protein includes an amino acid sequence having 95% or more sequence identity (e.g., 96% or more, 97% or more, 98% or more, 99% or more, 99.5% or more, or 100% sequence identity) with the amino acid sequence set forth in any one of SEQ ID NOs: 1-27. Also provided are nucleic acids, AAV vectors, viral particles, cells, kits, and methods.

### CROSS REFERENCE

This application claims the benefit of U.S. Provisional Patent Application No. 62/893,723 filed Aug. 29, 2019, which application is incorporated herein by reference in its entirety.

### STATEMENT REGARDING FEDERALLY SPONSORED RESEARCH

This invention was made with Government support under contract A1116698 awarded by the National Institutes of Health. The Government has certain rights in the invention.

### INCORPORATION BY REFERENCE OF SEQUENCE LISTING PROVIDED AS A TEXT FILE

A Sequence Listing is provided herewith as a text file, “STAN-1540prv_SeqList_ST25.txt” created on Aug. 29, 2019 and having a size of 260 KB. The contents of the text file are incorporated by reference herein in their entirety.

### I. INTRODUCTION

Genetic disorders caused by absence of or a defect in a desirable gene (loss of function) or expression of an undesirable or defective gene (gain of function) lead to a variety of diseases. At present, adeno-associated virus (AAV) vectors are recognized as the gene transfer vectors of choice for therapeutic applications since they have the best safety and efficacy profile for the delivery of genes in vivo.

Adeno-associated virus (AAV), a member of the Parvovirus family, is a small nonenveloped, icosahedral virus with single-stranded linear DNA genomes of 4.7 kilobases (kb). AAV is assigned to the genus, Dependovirus, because the virus was discovered as a contaminant in purified adenovirus stocks (D. M. Knipe, P. M. Howley, Field's Virology, Lippincott Williams & Wilkins, Philadelphia, ed. Sixth, 2013). In its wild-type state, AAV depends on a helper virus—typically adenovirus—to provide necessary protein factors for replication, as AAV is naturally replication-defective. The 4.7-kb genome of AAV is flanked by two inverted terminal repeats (ITRs) that fold into a hairpin shape important for replication.

Being naturally replication-defective and capable of transducing nearly every cell type in the human body, AAV represents an ideal vector for therapeutic use in gene therapy or vaccine delivery. In its wild-type state, AAV's life cycle includes a latent phase during which AAV genomes, after infection, are site-specifically integrated into host chromosomes and an infectious phase during which, following either adenovirus or herpes simplex virus infection, the integrated genomes are subsequently rescued, replicated, and packaged into infectious viruses. When vectorized, the viral Rep and Cap genes of AAV are removed and provided in trans during virus production, making the ITRs the only viral DNA that remains (A. Vasileva, R. Jessberger, Nature reviews. Microbiology, 3, 837-847 (2005)). Rep and Cap are then replaced with an array of possible transfer vector configurations to perform gene addition or gene targeting. These vectorized recombinant AAVs (rAAVs) transduce both dividing and non-dividing cells, and show robust stable expression in quiescent tissues like skeletal muscle. The number of rAAV gene therapy clinical trials that have been completed or are ongoing to treat various inherited or acquired diseases is increasing dramatically as rAAV-based therapies increase in popularity. Similarly, in the clinical vaccine space, there have been numerous recent preclinical studies and one ongoing clinical trial using rAAV as a vector to deliver antibody expression cassettes in passive vaccine approaches for human/simian immunodeficiency virus (HIV/SIV), influenza virus, henipavirus, and human papilloma virus (HPV).

The properties of non-pathogenicity, broad host range of infectivity, including non-dividing cells, and potential site-specific chromosomal integration make AAV an attractive tool for gene transfer. A variety of published US applications describe AAV vectors and virions, including U.S. Publication Nos. 2015/0176027, 2015/0023924, 2014/0348794, 2014/0242031, and 2012/0164106; all of which are incorporated by reference herein in their entireties.

The development of targeted gene therapy in the central nervous system (CNS) is important for advancing new therapeutic approaches to treat neurological disorders. The non-pathogenic adeno-associated virus (AAV) vector has emerged with high potential for in vivo gene delivery. A recent clinical trial using AAV9 to deliver survival motor neuron gene has shown unprecedented positive results in treating children with spinal muscular atrophy albeit very high dosing is required. Despite these encouraging developments in gene therapy, gene delivery to the CNS is still exceedingly difficult due to the biological transport barriers. For example, the blood-brain barrier (BBB) blocks intravenously injected vectors from entering the CNS, resulting in a large amount of gene transfer into peripheral tissues such as the liver. Furthermore, preclinical modeling with rAAV to determine the best capsid serotypes for transducing target tissues is done in animal models—typically mice—which do not necessarily recapitulate the tissue and cell tropism each rAAV has in humans, nor the transduction capabilities at treatment.

Provided herein are compositions and methods that address these limitations.

### II. SUMMARY

The present disclosure provides variant adeno-associated virus (AAV) capsid polypeptides that provide an AAV particle with the ability to traverse the human blood brain barrier (BBB) and transduce cells of the central nervous system (CNS) (e.g., astrocytes, neurons). In some embodiments the variant AAV capsid protein is referred to as a recombinant variant AAV (rAAV) capsid protein. In some cases, a subject variant AAV capsid protein includes an amino acid sequence having 95% or more sequence identity (e.g., 96% or more, 97% or more, 98% or more, 99% or more, 99.5% or more, or 100% sequence identity) with the amino acid sequence set forth in any one of SEQ ID NOs: 1-27. In some cases, a subject variant AAV capsid protein includes an amino acid sequence having 95% or more sequence identity (e.g., 96% or more, 97% or more, 98% or more, 99% or more, 99.5% or more, or 100% sequence identity) with the amino acid sequence set forth in any one of SEQ ID NOs: 1-4, 6-10, 12-24, and 27. In some cases, a subject variant AAV capsid protein includes an amino acid sequence having 80% or more sequence identity (e.g., 85% or more, 90% or more, 95% or more, 96% or more, 97% or more, 98% or more, 99% or more, or 99.5% or more sequence identity) with the amino acid sequence set forth in any one of SEQ ID NOs: 1-27, and the variant AAV capsid polypeptide includes at least one amino acid difference (e.g., amino acid substitution, amino acid insertion, amino acid deletion) relative to a substantially identical wild type AAV capsid protein.

The present disclosure provides nucleic acids (e.g., AAV vectors) comprising a nucleotide sequence coding a variant AAV capsid polypeptide that provides for (i.e., exhibits) the ability to cross the human BBB. In some embodiments the nucleic acid is an AAV vector and is referred to as a recombinant AAV or rAAV vector. In some cases a subject nucleic acid also includes a nucleotide sequence of interest (e.g., in some cases flanked by inverted terminal repeat sequences (ITRs)). The present disclosure also provides cells that include a subject nucleic acid.

The present disclosure provides recombinant AAV (rAAV) particles that include a subject variant AAV capsid protein and a nucleic acid payload of interest. In some cases the nucleic acid payload of interest encodes a protein (e.g., a genome-editing enzyme, a therapeutic protein, and the like) and in some cases the nucleic acid payload of interest encodes a non-coding RNA (e.g., an shRNA, a miRNA, an aptamer, a ribozyme, an antisense RNA, a CRISPR/Cas guide RNA, and the like). Also provided are cells that include a subject rAAV particle.

The present disclosure provides methods of delivering a payload of interest to the central nervous system of an individual. In some cases such methods include systemically administering (e.g., parenteral administration, intravenous administration, and the like) a subject rAAV particle to the individual.

### III. BRIEF DESCRIPTION OF THE DRAWINGS

FIG. 1. AAV Gene Therapy for Central Nervous System. (top left)-Schematic drawing of human brain and nerves. (top middle) Schematic drawing of the blood brain barrier (BBB). (top right)-Schematic drawing of transwell BBB model system used in the studies described herein. (bottom left graph)-Permeability of 2M MW dextran across hCMEC/D3 cells was not detectable. (bottom right graph)-Permeability of AAV9 in hCMEC/D3 cells is better than AAV2.

FIG. 2a-2b. 18 natural AAV capsids were tested for their permeability in the Transwell blood brain barrier (BBB) Model. These viruses included AAV9 and AAV-rhesus10 (red boxes), which were known to cross the BBB efficiently. The depicted results show the composition of the viruses in the input and the flowthrough crossing the Transwell BBB Model in FIG. 2a and FIG. 2b. 10 of these natural AAV caspids were chosen to create a shuffled capsid library for selection in the Transwell BBB Model.

FIG. 3a-3b. depict viral genomic structure (FIG. 3a) and the assay/selection scheme (FIG. 3b) that was used to test/screen a shuffled capsid AAV library in the Transwell BBB Model.

FIG. 4a-4c. (FIG. 4a) Viral Genome in Astrocytes and Flowthrough after crossing the Transwell BBB Model-5 of them (RS.N8d, RS.R3, RS.R6, RS.R11 and RS.R18) performed better than AAV9 and Rh10 in crossing the transwell, while 2 of them (RS.R4 and RS.R5) were similar to the two controls (AAV9 and rh10). RS.A5a1, RS.A5a2, RS.A5e, RS.A6, RS.A7 and RS.A8) also crossed the transwell at varying levels and transduced astrocytes at higher efficiency than controls AAV3B and LK03. (FIG. 4b and FIG. 4c) Luciferase Activity (a readout for transduction efficiency) in hCMEC/D3 Cells and Astrocytes. All of the tested viruses showed higher transduction efficiency in hCMEC cells and astrocytes than the AAV9 control virus. AAVs selected from astrocytes (A5-A8) showed increased transduction in astrocytes, and reduced transduction in hCMED/D3 cells. The ‘best’ vector was A6 (RS.A6), which performed more than 10-fold better than AAV-3B and LK03 in astrocytes. AAVs selected from neurons (RS.N8d and RS.R5) exhibited low transduction in hCMEC/D3 and astrocytes as anticipated based on the other results presented herein.

FIG. 5a-5e. Testing Transduction in human iPS Neurons and Astrocytes (72hpt). Variant viruses were tested for transduction efficiency in various different cell types: neurons derived from iPS cells (FIG. 5a), astrocytes derived from iPS cells (FIG. 5a), iPS generated neurons (FIG. 5a), 293T cells (FIG. 5b), 2 day old mouse cortex cells (FIG. 5b), non-differentiated (FIG. 5c) (epithelial cell-like) and differentiated (FIG. 5c) (neuron-like) SHSY5Y cells, neurons derived from iPS cells (FIG. 5d), and astrocytes derived from iPS cells (FIG. 5d). The transduction trend was very similar in every cell type: the tested variants performed better than AAV9 (control), but were slightly less efficient than AAV-DJ (control). RS.N8d was not efficient at transducing any of the tested cell types. FIG. 5e includes a summary of results at different multiplicity of infections (MOIs).

FIG. 6a-6b. Testing Transduction in Mice after Retro-Orbital Injection. The results (3 weeks after injection for FIGS. 6a and 30 days after injection for FIG. 6b) showed that none of the variants (with the exception of RS.R6) transduced mouse brain as efficiently as AAV9 control, and variants transduced the mouse brain less than AAV9-PHP.B (which is one of the best currently known in mice). However, this was not surprising because the viruses were selected using a model of the human system—and it was likely that they would not be very efficient in mice. This is similar to AAV9-PHP.B, which was selected in C57BL/6 mice, and is only efficient in C57BL/6 mice and not in other mouse strains or in non-human primates.

FIG. 7. Depicts results from non-human primate antibody neutralization assays. To test which non-human primate (NHP) will be promising/appropriate for pre-clinical NHP virus testing (e.g., won't mount a significant immune response against the introduced virus), the ability for antibodies in NHP serum to neutralize viruses identified in the screens discussed above was tested. The results indicated that in some NHP serum, all viruses (including AAV-DJ and AAV9 controls) were neutralized, and in others none of the viruses were neutralized. Note: these data do not speak to the transduction efficiency, but instead show that in at least some NHP serums the viruses were not neutralized by existing antibodies.

FIG. 8. Depicts a crossover (Xover) analysis of 6 viruses selected from the Astrocytes with Ad5 selection. RS.A5a1, RS.A5a2 and RS.A5e are originally the same virus in the selection, their differences are caused by PCR artifacts during sequencing sample preparation. All A5 viruses as well as A6 and A7 have similar parts in the red box, partially from AAVrh.10 parent and partially from AAV3B/LK03 parent, we hypothesize that this is what is required for the virus to be able to cross the endothelial cells, enter astrocytes, as well as cross in to the flowthough. A8, on the other hand, does not have the AAVrh.10 contribution right before AAV3B/LK03, and thus performs very similar to AAV3B/LK03 in the transwell in entering the astrocytes, but does not cross into the flowthrough.

FIG. 9a-9f. Depict a crossover (Xover) analysis of sequences selected in neurons. (FIG. 9a) This and the following figures show the Xover pattern analysis of the 19 sequences that were found that highly increased in the selection in neurons with replication in a preliminary PacBio sequence analysis. The total sequence actually is 21, but 2 of the sequences have 2 isoforms (labeled as R12a and R12b, and R13a and R13b in the figure) due to PCR artifacts. The sequences are arranged as labeled in FIG. 9a and FIG. 9f) by which parent contributed the most in the C-terminal of the virus sequences. Certain parental contributions appeared with different probability in different parts of the viruses [FIG. 9b: 12/19 had AAV3B at ˜aa530-610; FIG. 9c: 5/19 had AAV2+AAV3B at ˜aa450-610; FIG. 9d: 5/19 had AAVrh10 at ˜aa450-500; FIG. 9e: 6/19 had AAVrh10 at ˜aa200]. FIG. 9f depicts a summary of the previous figures (all 19 viruses selected from Neurons with Ad5 selection). The circles/arrows refer to how they performed in the transwell assay, and boxes show which parent contribution may have been responsible for their phenotype. 8 of the sequences with varying patterns were picked for the analyses described in the previous figures. Conclusions: (1) 4 crossed BBB more efficient than AAV9, 2 crossed BBB similar to AAV9, 1 crossed BBB less efficient AAV9, and 1 did not produce high virus titer; (2) AAV regions that may facilitate crossing BBB: AAV2 (˜aa450-550)+AAV3B (˜aa550-610).

FIG. 10a-10i. Amino acid of the generated/identified variant adeno-associated virus (AAV) capsid proteins that provide the ability to traverse the human blood brain barrier (BBB) and transduce cells of the CNS.

### IV. DEFINITIONS

“AAV” is an abbreviation for adeno-associated virus, and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”).

The term “AAV” includes AAV type 1 (AAV1), AAV type 2 (AAV2), AAV type 3 (AAV3), AAV type 4 (AAV4), AAV type 5 (AAVS), AAV type 6 (AAV6), AAV type 7 (AAV7), AAV type 8 (AAV8), AAV type 9 (AAV9), AAV 9_hu14, avian AAV, bovine AAV, canine AAV, equine AAV, primate AAV, non-primate AAV, and ovine AAV. “Primate AAV” refers to AAV capable of infecting primates, “non-primate AAV” refers to AAV capable of infecting non-primate mammals, “bovine AAV” refers to AAV capable of infecting bovine mammals, etc.

An “AAV vector” as used herein refers to a nucleic acid sequence encoding a variant capsid polypeptide (i.e., the AAV vector comprises a nucleic acid sequence encoding a variant capsid polypeptide, also referred to as a variant AAV capsid protein or variant AAV capsid polypeptide — the terms “polypeptide” and “protein” are used interchangeably herein), wherein the variant AAV capsid polypeptide exhibits (provides for) the ability to traverse the human blood brain barrier (BBB) (e.g., increased traversal of the human BBB as compared to a non-traversing wild type AAV such as AAV2 or AAV3B) and transduce cells of the CNS. The AAV vectors can also comprise a heterologous nucleic acid sequence not of AAV origin (e.g., as part of the nucleic acid insert). This heterologous nucleic acid sequence typically comprises a sequence of interest for the genetic transformation of a cell. In some cases, the heterologous nucleic acid sequence (the “nucleotide sequence of interest”) is flanked by at least one, and generally by two AAV inverted terminal repeat sequences (ITRs).

The phrase “non-variant parent capsid polypeptides” (or “wild type capsid protein”) includes any naturally occurring AAV capsid polypeptides. In some embodiments, the non-variant parent capsid polypeptides include AAV1, AAV2, AAV3, AAV4, AAV5, AAV6, AAV7, AAV8, AAV9, bovine AAV and/or avian AAV capsid polypeptides.

The term “substantially identical” in the context of variant AAV capsid polypeptides and non-variant parent capsid polypeptides refers to sequences with 1 or more amino acid changes. In some embodiments, these changes do not affect the packaging function of the capsid polypeptides. In some embodiments, substantially identical include variant AAV capsid polypeptides about 99%, about 98%, about 97%, about 96%, about 95%, about 94%, about 93%, about 92%, about 91%, or about 90% identical to non-variant parent capsid polypeptides. In some embodiments, the variant AAV capsid polypeptides can be substantially identical to non-variant parent capsid polypeptides over a subregion of the variant AAV capsid polypeptide, such as over about 25%, about 50%, about 75%, or about 90% of the total polypeptide sequence length.

An “AAV virion” or “AAV virus” or “AAV viral particle” or “AAV vector particle” refers to a viral particle composed of at least one AAV capsid polypeptide (including both variant AAV capsid polypeptides and non-variant parent capsid polypeptides) and an encapsidated polynucleotide AAV transfer vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle.

“Packaging” refers to a series of intracellular events resulting in the assembly of AAV virions or AAV particles which encapsidate a nucleic acid sequence and/or other therapeutic molecule. Packaging can refer to encapsidation of nucleic acid sequence and/or other therapeutic molecules into a capsid comprising the variant AAV capsid polypeptides described herein.

The phrase “therapeutic molecule” as used herein can include nucleic acids (including, for example, vectors), polypeptides (including, for example, antibodies), and vaccines, as well as any other therapeutic molecule that could be packaged by the variant AAV capsid polypeptides of the invention.

AAV “rep” and “cap” genes refer to polynucleotide sequences encoding replication and encapsidation proteins of adeno-associated virus (AAV). AAV rep (replication) and cap (capsid) are referred to herein as AAV “packaging genes.”

A “helper virus” for AAV refers to a virus allowing AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used as a helper virus. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome allowing AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virion, virus or viral particle is one comprising a polynucleotide component deliverable into a cell tropic for the viral species. The term does not necessarily imply any replication capacity of the virus. As used herein, an “infectious” virus or viral particle is one that upon accessing a target cell, can infect a target cell, and can express a heterologous nucleic acid in a target cell. Thus, “infectivity” refers to the ability of a viral particle to access a target cell, enter a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA) or fluorescence-activated cell sorting (FACS).

A “replication-competent” virion or virus (e.g. a replication-competent AAV) refers to an infectious phenotypically wild-type virus, and is replicable in an infected cell (i.e. in the presence of a helper virus or helper virus functions). In the case of AAV, replication competence generally requires the presence of functional AAV packaging genes. In some embodiments, AAV vectors, as described herein, lack of one or more AAV packaging genes and are replication-incompetent in mammalian cells (especially in human cells). In some embodiments, AAV vectors lack any AAV packaging gene sequences, minimizing the possibility of generating replication competent AAV by recombination between AAV packaging genes and an incoming AAV vector. In many embodiments, AAV vector preparations as described herein are those containing few if any replication competent AAV (rcAAV, also referred to as RCA) (e.g., less than about 1 rcAAV per 10.sup.2 AAV particles, less than about 1 rcAAV per 10.sup.4 AAV particles, less than about 1 rcAAV per 10.sup.8 AAV particles, less than about 1 rcAAV per 10.sup.12 AAV particles, or no rcAAV).

The terms “polynucleotide” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, IncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, IncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “small interfering” or “short interfering RNA” or siRNA is a RNA duplex of nucleotides targeted to a gene interest (a “target gene”). An “RNA duplex” refers to the structure formed by the complementary pairing between two regions of a RNA molecule. siRNA is “targeted” to a gene and the nucleotide sequence of the duplex portion of the siRNA is complementary to a nucleotide sequence of the targeted gene. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19-25 base pairs in length. The RNA duplex portion of the siRNA can be part of a hairpin structure. In addition to the duplex portion, the hairpin structure may contain a loop portion positioned between the two sequences forming the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

“Recombinant,” as applied to a polynucleotide means the polynucleotide is the product of various combinations of cloning, restriction or ligation steps, and other procedures resulting in a construct distinct and/or different from a polynucleotide found in nature. A recombinant virus is a viral particle encapsidating a recombinant polynucleotide. The terms respectively include replicates of the original polynucleotide construct and progeny of the original virus construct.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription usually downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a sequence of interest (the sequence of interest can also be said to be operatively linked to the promoter) if the promoter helps initiate transcription of the sequence of interest. There may be intervening residues between the promoter and sequence of interest so long as this functional relationship is maintained.

“Heterologous” means derived from a genotypically distinct entity from the rest of the entity to it is being compared too. For example, a polynucleotide introduced by genetic engineering techniques into a plasmid or vector derived from a different species is a heterologous polynucleotide. A promoter removed from its native coding sequence and operatively linked to a coding sequence it is not naturally found linked to a heterologous promoter. For example, an AAV including a heterologous nucleic acid encoding a heterologous gene product is an AAV including a nucleic acid not normally included in a naturally-occurring, wild-type AAV, and the encoded heterologous gene product is a gene product not normally encoded by a naturally-occurring, wild-type AAV. An AAV including a nucleic acid encoding a variant AAV capsid polypeptide includes a heterologous nucleic acid sequence. Once transferred/delivered into a host cell, a heterologous polynucleotide, contained within the virion, can be expressed (e.g., transcribed, and translated if appropriate). Alternatively, a transferred/delivered heterologous polynucleotide into a host cell, contained within the virion, need not be expressed. Although the term “heterologous” is not always used herein in reference to polynucleotides, reference to a polynucleotide even in the absence of the modifier “heterologous” is intended to include heterologous polynucleotides in spite of the omission.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or polynucleotide-liposome complexation. Genetic alteration may also be effected, for example, by transduction or infection with a DNA or RNA virus or viral vector. Generally, the genetic element is introduced into a chromosome or mini-chromosome in the cell; but any alteration changing the phenotype and/or genotype of the cell and its progeny is included in this term.

A cell is said to be “stably” altered, transduced, genetically modified, or transformed with a genetic sequence if the sequence is available to perform its function during an extended period of time (e.g., extended culture of the cell when the cell is in vitro). Such a cell can be “heritably” altered (genetically modified) in that a genetic alteration is introduced and can be inherited by progeny of the altered cell.

The terms “polypeptide,” “peptide,” and “protein” are used interchangeably herein to refer to polymers of amino acids of any length. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of the intended functionality. The terms also encompass a modified amino acid polymer; for example, disulfide bond formation, glycosylation, lipidation, phosphorylation, methylation, carboxylation, deamidation, acetylation, or conjugation with a labeling component. Polypeptides such as anti- angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, retaining the desired biochemical function of the intact protein.

An “isolated” plasmid, nucleic acid, vector, virus, virion, host cell, or other substance refers to a preparation of the substance devoid of at least some of the other components present where the substance or a similar substance naturally occurs or from which it is initially prepared. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

By the term “highly conserved” is meant at least about 80% identity, preferably at least 90% identity, and more preferably, over about 97% identity. Identity is readily determined by one of skill in the art by resort to algorithms and computer programs known by those of skill in the art.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The terms “pharmaceutically acceptable” and “physiologically acceptable” mean a biologically acceptable formulation, gaseous, liquid or solid, or mixture thereof, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering an AAV vector or AAV virion as disclosed herein, or transformed cell to a subject.

The phrase a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. AAV vectors or AAV virions, and pharmaceutical compositions thereof can be packaged in single or multiple unit dosage form for ease of administration and uniformity of dosage.

A “therapeutically effective amount” will fall in a relatively broad range determinable through experimentation and/or clinical trials. For example, for in vivo injection, e.g., injection directly into the tissue of a subject (for example, muscle tissue), a therapeutically effective dose will be on the order of from about 10⁶ to about 10¹⁵ of the AAV virions per kilogram bodyweight of the subject. In some embodiments, a therapeutically effective dose will be on the order of from about 10⁸ to 10¹² AAV virions per kilogram bodyweight of the subject. Other effective dosages can be readily established by one of ordinary skill in the art through routine trials establishing dose response curves.

An “effective amount” or “sufficient amount” refers to an amount providing, in single or multiple doses, alone or in combination, with one or more other compositions (therapeutic agents such as a drug), treatments, protocols, or therapeutic regimens agents (including, for example, vaccine regimens), a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is also a satisfactory outcome.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (protein), or producing an aberrant, partially functional or non-functional gene product (protein), leading to disease; and subjects screening positive for an aberrant, or defective (mutant) gene product (protein) leading to disease, even though such subjects do not manifest symptoms of the disease.

The phrases “tropism” and “transduction” are interrelated, but there are differences. The term “tropism” as used herein refers to the ability of an AAV vector or virion to infect one or more specified cell types, but can also encompass how the vector functions to transduce the cell in the one or more specified cell types; i.e., tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). As used herein, the term “transduction” refers to the ability of an AAV vector or virion to infect one or more particular cell types; i.e., transduction refers to entry of the AAV vector or virion into the cell and the transfer of genetic material contained within the AAV vector or virion into the cell to obtain expression from the vector genome. In some cases, but not all cases, transduction and tropism may correlate.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 110%, 125%, 150%, 175%, or 200% or more of the transduction or tropism, respectively, of the control). Suitable controls will depend on a variety of factors including the desired tropism profile. Similarly, it can be determined if a capsid and/or virus “does not efficiently transduce” or “does not have efficient tropism” for a target tissue, or similar terms, by reference to a suitable control.

Unless indicated otherwise, “efficient traversal” of the BBB, or similar terms, can be determined by reference to a suitable control (e.g., at least about 50%, 60%, 70%, 80%, 85%, 90%, 95%, 100%, 110%, 125%, 150%, 175%, or 200% or more of the traversal, respectively, of the control). Suitable controls will depend on a variety of factors including the desired traversal profile. Similarly, it can be determined if a capsid and/or virus “does not efficiently traverse” or “does not traverse” the human BBB, by reference to a suitable control. In some cases, the control will be a wild type AAV with a wild type AAV capsid protein, where the wild type AAV is considered to NOT traverse the BBB—one such example is AAV2. In some cases, the control will be a wild type AAV with a wild type AAV capsid protein, where the wild type AAV can traverse the BBB—one such example is AAV9. Thus, a subject variant AAV capsid protein provides for increased traversal of the human BBB compared to AAV2. In some cases, a subject variant AAV capsid protein provides for traversal of the human BBB, but the traversal is comparable to (e.g., from 80%-120% of, 80% of, 100% of, or 120% of) the traversal of a control such as AAV9. In some cases, a subject variant AAV capsid protein provides for increased traversal of the human BBB (e.g., 1.1-fold or more, 1.2-fold or more, 1.5-fold or more, 1.7 fold or more, 2-fold or more, 2.5-fold or more, 5-fold or more, 10-fold or more, etc.), even when compared to a control wild type (e.g., AAV9) that also provides for traversal of the human BBB.

### V. DETAILED DESCRIPTION

As noted above, the present disclosure provides variant adeno-associated virus (AAV) capsid polypeptides that provide an AAV particle with the ability to traverse the human blood brain barrier (BBB) and transduce cells of the central nervous system. In some embodiments the variant AAV capsid protein is referred to as a recombinant variant AAV (rAAV) capsid protein. In some cases, a subject variant AAV capsid protein includes an amino acid sequence having 95% or more sequence identity (e.g., 96% or more, 97% or more, 98% or more, 99% or more, 99.5% or more, or 100% sequence identity) with the amino acid sequence set forth in any one of SEQ ID NOs: 1-27. In some cases, a subject variant AAV capsid protein includes an amino acid sequence having 80% or more sequence identity (e.g., 85% or more, 90% or more, 95% or more, 96% or more, 97% or more, 98% or more, 99% or more, or 99.5% or more sequence identity) with the amino acid sequence set forth in any one of SEQ ID NOs: 1-27, and the variant AAV capsid polypeptide includes at least one amino acid difference (e.g., amino acid substitution, amino acid insertion, amino acid deletion) relative to a substantially identical wild type AAV capsid protein.

The present disclosure provides nucleic acids (e.g., AAV vectors) comprising a nucleotide sequence coding a variant AAV capsid polypeptide that provides for (i.e., exhibits) the ability to cross the human BBB and transduce cells of the central nervous system. In some embodiments the nucleic acid is an AAV vector and is referred to as a recombinant AAV or rAAV vector. In some cases a subject nucleic acid also includes a nucleotide sequence of interest (e.g., in some cases flanked by inverted terminal repeat sequences (ITRs)). The present disclosure also provides cells that include a subject nucleic acid.

The present disclosure provides recombinant AAV (rAAV) particles that include a subject variant AAV capsid protein and a nucleic acid payload of interest. In some cases the nucleic acid payload of interest encodes a protein (e.g., a genome-editing enzyme, a therapeutic protein, and the like) and in some cases the nucleic acid payload of interest encodes a non-coding RNA (e.g., an shRNA, a miRNA, an aptamer, a ribozyme, an antisense RNA, a CRISPR/Cas guide RNA, and the like). Also provided are cells that include a subject rAAV particle.

The present disclosure provides methods of delivering a payload of interest to the central nervous system of an individual. In some cases such methods include systemically administering (e.g., parenteral administration, intravenous administration, and the like) a subject rAAV particle to the individual.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

AAV Capsid AND Vector Features

AAV vectors and proteins of the present disclosure have numerous features. In some embodiments, a subject vector comprises a nucleic acid sequence encoding a variant AAV capsid polypeptide. Such AAV vectors and their features are described in detail below.

A subject variant AAV capsid protein provides an AAV viral particle (an rAAV particle) with the ability to traverse (cross) the human blood brain barrier (BBB) (e.g., after systemic administration). In some cases, such a subject rAAV particle can then transduce neurons (e.g., neurons in the brain). In some cases, such a subject rAAV particle can then transduce astrocytes (e.g., astrocytes in the brain). In some cases, such a subject rAAV particle can transduce neurons and astrocytes (e.g., neurons and astrocytes in the brain).

In some cases, the ability to traverse the human BBB that is provided by a subject variant AAV capsid protein can be compared to that of a control AAV capsid protein. The control protein can be a wild type AAV capsid protein, where the wild type AAV is considered to NOT traverse the BBB—one such example is AAV2. In some cases, the control can be a wild type wild type AAV capsid protein, where the wild type AAV (having that capsid protein) can traverse the BBB—one such example is AAV9. A subject variant AAV capsid protein provides for increased traversal of the human BBB compared to a wild type capsid protein (such as the AAV2 capsid protein). In some cases, a subject variant AAV capsid protein provides for traversal of the human BBB, but the traversal is comparable to (e.g., from 80%-120% of, 80% of, 100% of, or 120% of) the traversal of a control wild type AAV that can traverse the human BBB (e.g., such as AAV9). In some cases, a subject variant AAV capsid protein provides for increased traversal of the human BBB (e.g., 1.1-fold or more, 1.2-fold or more, 1.5-fold or more, 1.7 fold or more, 2-fold or more, 2.5-fold or more, 5-fold or more, 10-fold or more, etc.), when compared to a control wild type (e.g., AAV9) that provides for traversal of the human BBB.

An example AAV vector of the present disclosure includes a nucleic acid encoding a variant AAV capsid protein differing in amino acid sequence by at least one amino acid from a wild-type (non-variant parent) capsid protein. The amino acid difference(s) can be located in a solvent accessible site in the capsid, e.g., a solvent-accessible loop, or in the lumen (i.e., the interior space of the AAV capsid). In some embodiments, the lumen includes the interior space of the AAV capsid. For example, the amino acid substitution(s) can be located in a GH loop in the AAV capsid polypeptide. In some embodiments, the variant AAV capsid polypeptide comprises an amino acid substitution in AAV1, AAV2, AAV3, AAV4, AAVS, AAV6, AAV7, AAV8, or AAV9 capsid polypeptides. In some cases, the variant AAV capsid protein is a shuffled variant, meaning that the variant AAV capsid protein resulted from the shuffling of multiple parent capsid protein sequences—and thus such a variant AAV capsid protein include stretches of wild type sequence, but the capsid protein sequence as a whole does not occur in nature.

In some embodiments, the present disclosure provides a nucleic acid comprising a nucleotide sequence that encodes a variant adeno-associated virus (AAV) capsid protein that comprises an amino acid sequence having at least about 85% at least about 90%, at least about 95%, at least about 98%, or at least about 99% amino acid sequence identity with a non-variant (wild type) capsid amino acid sequence, and provides a viral particle with the ability to traverse the human BBB.

The present disclosure provides a nucleic acid comprising a nucleotide sequence that encodes a variant adeno-associated virus (AAV) capsid protein (e.g., any of the variants described herein). In some embodiments, the variant AAV capsid polypeptide is a shuffled capsid protein that includes one or more regions or sub-portions from non-variant (wild type) parent capsid polypeptide sequences from AAV serotypes 1, 2, 3, 6, 8, and 9 (i.e., AAV1, AAV2, AAV3, AAV6, AAV8, and AAV9).

In some embodiments, a subject variant adeno-associated virus (AAV) capsid protein provides a viral particle with the ability to traverse the human blood brain barrier (BBB) and transduce cells of the central nervous system (CNS), where the variant AAV capsid protein comprises an amino acid sequence having AAV2 sequence in a region (e.g., amino acids 417-533, 447-519, 435-519, 447-533, 432-532, 417-524, about 415 to about 535, about 420 to about 530, about 445 to about 525, or about 450 to about 520) corresponding to amino acid position 445 to 518 of AAV2 (SEQ ID NO: 28) and AAV3B sequence in a region (e.g., amino acids 520-726, 534-602, 520-602, 534-726, 532-639, 525-725, about 515 to about 730, about 520 to about 730, about 520 to about 725, about 530 to about 600, about 530 to about 605, about 535 to about 600, or about 535 to about 605) corresponding to position 533 to 603 of AAV3B (SEQ ID NO: 29). As an illustrative example, for RS.R3 of the working examples: amino acids 435-519 come from AAV2 amino acids 434-518, and amino acids 520-602 come from AAV3B amino acids 520-602. For RS.R6 of the working examples: amino acids 447-533 come from AAV2 amino acids 445-531, and amino acids 534-726 come from AAV3B amino acids 533-725. For RS.R11 of the working examples: amino acids 432-532 come from AAV2 amino acids 431-531, and amino acids 532-639 come from AAV3B amino acids 532-639. For RS.R12a/b of the working examples: amino acids 417-524 come from AAV2 amino acids 416-523, and amino acids 525-725 come from AAV3B amino acids 525-725. Thus, the overlapping parts for these viruses is AAV2 amino acids 445-518 and AAV3B amino acids 533-602.

In some embodiments, a subject variant adeno-associated virus (AAV) capsid protein provides a viral particle with the ability to traverse the human blood brain barrier (BBB) and transduce cells of the central nervous system (CNS), where the variant AAV capsid protein comprises an amino acid sequence having AAV2 sequence in a region (e.g., amino acids 417-533, 447-519, 435-519, 447-533, 432-532, 417-524, about 415 to about 535, about 420 to about 530, about 445 to about 525, or about 450 to about 520) identical to amino acid position 445 to 518 of AAV2 (SEQ ID NO: 28) and AAV3B sequence in a region (e.g., amino acids 520-726, 534-602, 520-602, 534-726, 532-639, 525-725, about 515 to about 730, about 520 to about 730, about 520 to about 725, about 530 to about 600, about 530 to about 605, about 535 to about 600, or about 535 to about 605) identical to position 533 to 603 of AAV3B (SEQ ID NO: 29).

Many amino acids are shared between the parental AAVs—the unique amino acids in these regions are, for AAV2: P451, T456, 461Q, A467, D469, I470, S492, A493, and Y500; and for AAV3B: H538, N540, T549, E554, N582, T592, R594, and D598. Thus, in some embodiments, a subject variant adeno-associated virus (AAV) capsid protein provides a viral particle with the ability to traverse the human blood brain barrier (BBB) and transduce cells of the central nervous system (CNS), where the variant AAV capsid protein comprises an amino acid sequence that includes: (1) P451, T456, 461Q, A467, D469, I470, S492, A493, and Y500 from AAV2 (SEQ ID NO: 28) in a corresponding region (e.g., amino acids 417-533, 447-519, 435-519, 447-533, 432-532, 417-524, about 415 to about 535, about 420 to about 530, about 445 to about 525, or about 450 to about 520); and (2) H538, N540, T549, E554, N582, T592, R594, and D598 from AAV3B (SEQ ID NO: 29) in a corresponding region (e.g., amino acids 520-726, 534-602, 520-602, 534-726, 532-639, 525-725, about 515 to about 730, about 520 to about 730, about 520 to about 725, about 530 to about 600, about 530 to about 605, about 535 to about 600, or about 535 to about 605). In some cases, the variant AAV capsid protein comprises an amino acid sequence having AAV2 sequence in the region from about amino acid 450 to amino acid 550, and AAV3B sequence in the region from about amino acid 550 to amino acid 610.

In some cases, a variant AAV capsid protein comprises an amino acid sequence having 80% or more sequence identity (e.g., 85% or more, 90% or more, 95% or more, 96% or more, 97% or more, 98% or more, 99% or more, or 99.5% or more sequence identity) with the amino acid sequence set forth in any one of SEQ ID NOs: 1-27, and the variant AAV capsid polypeptide includes at least one amino acid difference (e.g., amino acid substitution, amino acid insertion, amino acid deletion) relative to a substantially identical wild type AAV capsid protein. In some cases, the variant AAV capsid protein comprises an amino acid sequence having 90% or more sequence identity (e.g., 95% or more, 96% or more, 97% or more, 98% or more, 99% or more, or 99.5% or more sequence identity) with the amino acid sequence set forth in any one of SEQ ID NOs: 1-27, and the variant AAV capsid polypeptide includes at least one amino acid difference (e.g., amino acid substitution, amino acid insertion, amino acid deletion) relative to a substantially identical wild type AAV capsid protein.

In some embodiments, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 1-27. For example, in some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 1-14. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 1-6. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 7-14. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 8-14. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 7 and 9-14. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 9-14. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 3, 4, and 11. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 15-27.

In some embodiments, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 1-27. For example, in some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 1-14. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 1-6. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 7-14. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 8-14. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 7 and 9-14. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 9-14. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 3, 4, and 11. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 15-27.

In some embodiments, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 1-27. For example, in some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 1-14. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 1-6. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 7-14. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 8-14. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 7 and 9-14. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 9-14. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 3, 4, and 11. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 15-27.

In some embodiments, the variant AAV capsid protein comprises an amino acid sequence having 98% or more (e.g., 99% or more, 99.5% or more, or 100%) sequence identity with the amino acid sequence set forth in any one of SEQ ID NOs: 1-27. For example, in some cases, the variant AAV capsid protein comprises an amino acid sequence having 98% or more (e.g., 99% or more, 99.5% or more, or 100%) sequence identity with the amino acid sequence set forth in any one of SEQ ID NOs: 1-14. In some cases, the variant AAV capsid protein comprises an amino acid sequence having 98% or more (e.g., 98% or more, 99% or more, 99.5% or more, or 100%) sequence identity with the amino acid sequence set forth in any one of SEQ ID NOs: 1-6. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 7-14. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 8-14. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 7 and 9-14. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 9-14. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 3, 4, and 11. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 15-27.

In some embodiments, the variant AAV capsid protein comprises an amino acid sequence having 99% or more (e.g., 99.5% or more, or 100%) sequence identity with the amino acid sequence set forth in any one of SEQ ID NOs: 1-27. For example, in some cases, the variant AAV capsid protein comprises an amino acid sequence having 99% or more (e.g., 99.5% or more, or 100%) sequence identity with the amino acid sequence set forth in any one of SEQ ID NOs: 1-14. In some cases, the variant AAV capsid protein comprises an amino acid sequence having 99% or more (e.g., 99.5% or more, or 100%) sequence identity with the amino acid sequence set forth in any one of SEQ ID NOs: 1-6. In some cases, the variant AAV capsid protein comprises an amino acid sequence having 99% or more (e.g., 99.5% or more, or 100%) sequence identity with the amino acid sequence set forth in any one of SEQ ID NOs: 7-14. In some cases, the variant AAV capsid protein comprises an amino acid sequence having 99% or more (e.g., 99.5% or more, or 100%) sequence identity with the amino acid sequence set forth in any one of SEQ ID NOs: 8-14. In some cases, the variant AAV capsid protein comprises an amino acid sequence having 99% or more (e.g., 99.5% or more, or 100%) sequence identity with the amino acid sequence set forth in any one of SEQ ID NOs: 7 and 9-14. In some cases, the variant AAV capsid protein comprises an amino acid sequence having 99% or more (e.g., 99.5% or more, or 100%) sequence identity with the amino acid sequence set forth in any one of SEQ ID NOs: 9-14. In some cases, the variant AAV capsid protein comprises an amino acid sequence having 99% or more (e.g., 99.5% or more, or 100%) sequence identity with the amino acid sequence set forth in any one of SEQ ID NOs: 3, 4, and 11. In some cases, the variant AAV capsid protein comprises an amino acid sequence having 99% or more (e.g., 99.5% or more, or 100%) sequence identity with the amino acid sequence set forth in any one of SEQ ID NOs: 15-27.

In some embodiments, the variant AAV capsid protein comprises the amino acid sequence set forth in any one of SEQ ID NOs: 1-27. For example, in some cases, the variant AAV capsid protein comprises the amino acid sequence set forth in any one of SEQ ID NOs: 1-14. In some cases, the variant AAV capsid protein comprises the amino acid sequence set forth in any one of SEQ ID NOs: 1-6. In some cases, the variant AAV capsid protein comprises the amino acid sequence set forth in any one of SEQ ID NOs: 7-14. In some cases, the variant AAV capsid protein comprises the amino acid sequence set forth in any one of SEQ ID NOs: 8-14. In some cases, the variant AAV capsid protein comprises the amino acid sequence set forth in any one of SEQ ID NOs: 7 and 9-14. In some cases, the variant AAV capsid protein comprises the amino acid sequence set forth in any one of SEQ ID NOs: 9-14. In some cases, the variant AAV capsid protein comprises the amino acid sequence set forth in any one of SEQ ID NOs: 3, 4, and 11. In some cases, the variant AAV capsid protein comprises the amino acid sequence set forth in any one of SEQ ID NOs: 15-27.

In some embodiments, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 8-9, 12-14, 15-24, and 27. For example, in some cases the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 8-9 and 12-14. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 9 and 12-14. In some cases, the variant AAV capsid protein comprises an amino acid sequence having 86% or more (e.g., 88% or more, 90% or more, 92% or more, 95% or more, 98% or more, 99% or more, 99.5% or more, or 100%) sequence identity with the amino acid sequence set forth in any one of SEQ ID NOs: 15-24, and 27.

In some embodiments, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 8-9, 12-14, 15-24, and 27. For example, in some cases the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 8-9 and 12-14. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 9 and 12-14. In some cases, the variant AAV capsid protein comprises an amino acid sequence having 95% or more (e.g., 98% or more, 99% or more, 99.5% or more, or 100%) sequence identity with the amino acid sequence set forth in any one of SEQ ID NOs: 15-24, and 27.

In some embodiments, the variant AAV capsid protein comprises an amino acid sequence having 95% or more (e.g., 96% for more, 97% or more, 98% or more, 99% or more, 99.5% or more, or 100%) sequence identity with the amino acid sequence set forth in any one of SEQ ID NOs: 8-9, 12-14, 15-24, and 27. For example, in some cases the variant AAV capsid protein comprises an amino acid sequence having 95% or more (e.g., 96% for more, 97% or more, 98% or more, 99% or more, 99.5% or more, or 100%) sequence identity with the amino acid sequence set forth in any one of SEQ ID NOs: 8-9 and 12-14. In some cases, the variant AAV capsid protein comprises an amino acid sequence having 95% or more (e.g., 96% for more, 97% or more, 98% or more, 99% or more, 99.5% or more, or 100%) sequence identity with the amino acid sequence set forth in any one of SEQ ID NOs: 9 and 12-14. In some cases, the variant AAV capsid protein comprises an amino acid sequence having 95% or more (e.g., 96% for more, 97% or more, 98% or more, 99% or more, 99.5% or more, or 100%) sequence identity with the amino acid sequence set forth in any one of SEQ ID NOs: 15-24, and 27.

In some embodiments, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 8-9, 12-14, 15-24, and 27. For example, in some cases the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 8-9 and 12-14. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 9 and 12-14. In some cases, the variant AAV capsid protein comprises an amino acid sequence having 97% or more (e.g., 98% or more, 99% or more, 99.5% or more, or 100%) sequence identity with the amino acid sequence set forth in any one of SEQ ID NOs: 15-24, and 27.

In some embodiments, the variant AAV capsid protein comprises an amino acid sequence having 98% or more (e.g., 99% or more, 99.5% or more, or 100%) sequence identity with the amino acid sequence set forth in any one of SEQ ID NOs: 8-9, 12-14, 15-24, and 27. For example, in some cases the variant AAV capsid protein comprises an amino acid sequence having 98% or more (e.g., 99% or more, 99.5% or more, or 100%) sequence identity with the amino acid sequence set forth in any one of SEQ ID NOs: 8-9 and 12-14. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 9 and 12-14. In some cases, the variant AAV capsid protein comprises an amino acid sequence having 98% or more (e.g., 99% or more, 99.5% or more, or 100%) sequence identity with the amino acid sequence set forth in any one of SEQ ID NOs: 15-24, and 27.

In some embodiments, the variant AAV capsid protein comprises an amino acid sequence having 99% or more (e.g., 99.5% or more, or 100%) sequence identity with the amino acid sequence set forth in any one of SEQ ID NOs: 8-9, 12-14, 15-24, and 27. For example, in some cases the variant AAV capsid protein comprises an amino acid sequence having 99% or more (e.g., 99.5% or more, or 100%) sequence identity with the amino acid sequence set forth in any one of SEQ ID NOs: 8-9 and 12-14. In some cases, the variant AAV capsid protein comprises an amino acid sequence having 99% or more (e.g., 99.5% or more, or 100%) sequence identity with the amino acid sequence set forth in any one of SEQ ID NOs: 9 and 12-14. In some cases, the variant AAV capsid protein comprises an amino acid sequence having 99% or more (e.g., 99.5% or more, or 100%) sequence identity with the amino acid sequence set forth in any one of SEQ ID NOs: 15-24, and 27.

In some embodiments, the variant AAV capsid protein comprises the amino acid sequence set forth in any one of SEQ ID NOs: 8-9, 12-14, 15-24, and 27. For example, in some cases the variant AAV capsid protein comprises the amino acid sequence set forth in any one of SEQ ID NOs: 8-9 and 12-14. In some cases, the variant AAV capsid protein comprises the amino acid sequence set forth in any one of SEQ ID NOs: 9 and 12-14. In some cases, the variant AAV capsid protein comprises the amino acid sequence set forth in any one of SEQ ID NOs: 15-24, and 27.

TABLE 1
Identified variant adeno-associated virus (AAV) capsid proteins (see the
working examples below) that provide viral particles with the ability to
traverse the human blood brain barrier (BBB) and transduce cells of the
CNS.

| | AAV | SEQ ID NO | |
|---|---|---|---|
| Isolate from: | variant | Protein | DNA |
| Astrocytes | RS.A5a1 | 1 | 31 |
| | RS.A5a2 | 2 | 32 |
| | RS.A5e | 3 | 33 |
| | RS.A6 | 4 | 34 |
| | RS.A7 | 5 | 35 |
| | RS.A8 | 6 | 36 |
| Neurons | RS.N2 | 7 | 37 |
| | RS.N8d | 8 | 38 |
| | RS.R3 | 9 | 39 |
| | RS.R4 | 10 | 40 |
| | RS.R5 | 11 | 41 |
| | RS.R6 | 12 | 42 |
| | RS.R11 | 13 | 43 |
| | RS.R18 | 14 | 44 |
| | RS.N1 | 15 | 45 |
| | RS.R9 | 16 | 46 |
| | RS.R10 | 17 | 47 |
| | RS.R12a | 18 | 48 |
| | RS.R12b | 19 | 49 |
| | RS.R13a | 20 | 50 |
| | RS.R13b | 21 | 51 |
| | RS.R14 | 22 | 52 |
| | RS.R15 | 23 | 53 |
| | RS.R17 | 24 | 54 |
| | RS.R19 | 25 | 55 |
| | RS.R22 | 26 | 56 |
| | RS.R24 | 27 | 57 |

Nucleotide Sequence of Interest

In some cases a subject nucleic acid, in addition to including a sequence that encodes a variant AAV capsid protein, also encodes a nucleic acid insert (also referred to as a heterologous nucleotide sequence or the “nucleotide sequence of interest”). Likewise, in some cases a subject rAAV particle, in addition to including a variant AAV capsid protein, also includes (e.g., encapsidates) a nucleic acid payload of interest (which includes a nucleotide sequence of interest). The “nucleotide sequence of interest can be operably linked to control elements directing the transcription or expression thereof once the sequence is present inside of a cell (e.g., in some cases integrated into the cell's genome). Such control elements can comprise control sequences normally associated with the selected gene (e.g., endogenous cellular control elements). Alternatively, heterologous control sequences can be employed. Useful heterologous control sequences generally include those derived from sequences encoding mammalian or viral genes. Examples include, but are not limited to, the SV40 early promoter, mouse mammary tumor virus long terminal repeat (LTR) promoter; adenovirus major late promoter (Ad MLP); a herpes simplex virus (HSV) promoter, an endogenous cellular promoter heterologous to the gene of interest, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, can also be used. Such promoter sequences are commercially available from, e.g., Stratagene (San Diego, Calif.).

In some embodiments, a cell type-specific or a tissue-specific promoter can be operably linked to the nucleotide sequence of interest and allowing for selective or preferential expression in a particular cell type(s) or tissue(s). Thus, in some embodiments, an inducible promoter can be operably linked to the nucleotide sequence of interest.

In some embodiments, a nucleic acid payload is packaged with the variant AAV capsid polypeptides of the disclosure. In some embodiments, the nucleic acid payload is at least 50, 100, 200, 300, 400, 500, 600, 700, 800, 900, 1000, 1100, 1200, 1300, 1400, or 1500 nucleotides (nt) in length. In some embodiments, the nucleic acid payload is 50 nucleotides to 4000 nucleotides long (e.g., 50-3000, 50-2000, 50-1500, 50-1200, 50-1000, 50-900, 50-750, 50-500, 100-4000, 100-3000, 100-2000, 100-1500, 100-1200, 100-1000, 100-900, 100-750, 100-500, 300-4000, 300-3000, 300-2000, 300-1500, 300-1200, 300-1000, 300-900, 300-750, 300-500, 500-4000, 500-3000, 500-2000, 500-1500, 500-1200, 500-1000, or 500-900 nt long). In some embodiments, the nucleotide sequence of interest is at least 50, 100, 200, 300, 400, 500, 600, 700, 800, 900, 1000, 1100, 1200, 1300, 1400, or 1500 nucleotides (nt) in length. In some embodiments, the nucleotide sequence of interest is 50 nucleotides to 4000 nucleotides long (e.g., 50-3000, 50-2000, 50-1500, 50-1200, 50-1000, 50-900, 50-750, 50-500, 100-4000, 100-3000, 100-2000, 100-1500, 100-1200, 100-1000, 100-900, 100-750, 100-500, 300-4000, 300-3000, 300-2000, 300-1500, 300-1200, 300-1000, 300-900, 300-750, 300-500, 500-4000, 500-3000, 500-2000, 500-1500, 500-1200, 500-1000, or 500-900 nt long).

In some embodiments, an AAV vector packaged by a variant AAV capsid polypeptide is at least about 2000 nucleotides in total length and up to about 5000 nucleotides in total length. In some embodiments, an AAV vector packaged by the variant AAV capsid polypeptides is about 2000 nucleotides, about 2400 nucleotides, about 2800 nucleotides, about 3000 nucleotides, about 3200 nucleotides, about 3400 nucleotides, about 3600 nucleotides, about 3800 nucleotides, about 4000 nucleotides, about 4200 nucleotides, about 4400 nucleotides, about 4600 nucleotides, about 4700 nucleotides, or about 4800 nucleotides. In some embodiments, an AAV vector packaged by the variant AAV capsid polypeptides is between about 2000 nucleotides (2 kb) and about 5000 nucleotides (5 kb). In some embodiments, an AAV vector packaged by the variant AAV capsid polypeptides is between about 2400 nucleotides (2.4 kb) and about 4800 nucleotides (4.8 kb). In some embodiments, an AAV vector packaged by the variant AAV capsid polypeptides is between about 3000 nucleotides (3 kb) and about 5000 nucleotides (5 kb). In some embodiments, an AAV vector packaged by the variant AAV capsid polypeptides is between about 3000 nucleotides (3 kb) and about 4000 nucleotides (4 kb).

The AAV vectors or AAV virions disclosed herein can also include conventional control elements operably linked to the nucleic acid insert (also referred to as a heterologous nucleotide sequence or a “nucleotide sequence of interest”) in a manner permitting transcription, translation and/or expression in a cell transfected with the AAV vector or infected with the AAV virion produced according to the present disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters selected from native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the beta-actin promoter, the phosphoglycerol kinase (PGK) promoter, and the EF1 promoter (Invitrogen). Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clonetech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., (1996) Proc. Natl. Acad. Sci. USA, 93:3346-3351), the tetracycline-repressible system (Gossen et al., (1992) Proc. Natl. Acad. Sci. USA, 89:5547-5551), the tetracycline-inducible system (Gossen et al., (1995) Science, 268:1766-1769, see also Harvey et al., (1998) Curr. Opin. Chem. Biol., 2:512-518), the RU486-inducible system (Wang et al., (1997) Nat. Biotech., 15:239-243 and Wang et al., (1997) Gene Ther., 4:432-441) and the rapamycin-inducible system (Magari et al., (1997) J. Clin. Invest., 100:2865-2872). Other types of inducible promoters useful in this context are those regulated by a specific physiological state, e.g., temperature, acute phase, a particular differentiation state of the cell, or in replicating cells only.

In some cases a nucleotide sequence of interest is operably linked to a tissue-specific promoter. For instance, if expression in skeletal muscle is desired, a promoter active in muscle should be used. These include the promoters from genes encoding skeletal .beta.-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., (1997) J. Virol., 71:5124-32; hepatitis B virus core promoter, Sandig et al., (1996) Gene Ther., 3:1002-9; alpha-fetoprotein (AFP), Arbuthnot et al., (1996) Hum. Gene Ther., 7:1503-14), bone osteocalcin (Stein et al., (1997) Mol. Biol. Rep., 24:185-96); bone sialoprotein (Chen et al., (1996) J. Bone Miner. Res., 11:654-64), lymphocytes (CD2, Hansal et al., (1998) J. Immunol., 161:1063-8; immunoglobulin heavy chain; T cell receptor chain), neuronal such as neuron-specific enolase (NSE) promoter (Andersen et al., (1993) Cell. Mol. Neurobiol., 13:503-15), neurofilament light-chain gene (Piccioli et al., (1991) Proc. Natl. Acad. Sci. USA, 88:5611-5), and the neuron-specific vgf gene (Piccioli et al., (1995) Neuron, 15:373-84), among others.

In various embodiments, AAV vectors or AAV virions carrying one or more therapeutically useful nucleic acid inserts (also referred to as a heterologous nucleotide sequences or “nucleotide sequences of interest”) also include selectable markers or reporter genes, e.g., sequences encoding geneticin, hygromycin or puromycin resistance, among others. Selectable reporters or marker genes can be used to signal the presence of the plasmids/vectors in bacterial cells, including, for example, examining ampicillin resistance. Other components of the plasmid may include an origin of replication. Selection of these and other promoters and vector elements are conventional and many such sequences are available (see, e.g., Sambrook et al., and references cited therein).

In some cases a subject nucleotide sequence of interest encodes a non-coding RNA (e.g., a CRISPR/Cas guide RNA, an antisense RNA, a ribozyme, an shRNA, a microRNA, an aptamer). In some cases a subject nucleotide sequence of interest encodes a protein (e.g., a therapeutic protein meant to alleviate a disease and/or its symptoms, a genome-editing enzyme such as a CRISPR/Cas effector protein, TALEN, Zinc Finger nuclease, etc.—meant to provide for targeted genome editing, etc.). Examples of peptide or polypeptides envisioned as having a therapeutic activity for the multicellular organism in which they are expressed (e.g., via a nucleic acid encoding the peptide or polypeptide) include, but are not limited to: factor VIII, factor IX, β-globin, a CRISPR/Cas effector protein (e.g., Cas9, Cpf1, and the like), a low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, PDGF, VEGF, EGF, TGFα, TGBβ, FGF, TNF, IL-1, IL-2, IL-6, IL-8, endothelium derived growth factor (EDGF), ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase; a neuroprotective factor, e.g. a neurotrophin (e.g. NGF, BDNF, NT-3, NT-4, CNTF), Kifap3, Bcl-xl, collapsin response mediator protein 1, Chkβ, calmodulin 2, calcyon, NPT1, Eef1a1, Dhps, Cd151, Morf412, CTGF, LDH-A, Atl1, NPT2, Ehd3, Cox5b, Tuba1a, [symbol] -actin, Rpsa, NPG3, NPG4, NPG5, NPG6, NPG7, NPG8, NPG9, NPG10, dopamine, interleukins, cytokines, small peptides, the genes/proteins listed in Table 1 (see below: BCKDH complex (E1a, E1b and E2 subunits); Methylmalonyl-CoA Mutase; Propionyl-CoA Carboxylase (Alpha and Beta subunits); Isovaleryl CoA dehydrogenase; HADHA; HADHB; LCHAD; ACADM; ACADVL; G6PC (GSD1a); G6PT1(GSD1b); SLC17A3; SLC37A4 (GSD1c); Acid alpha-glucosidase; OCTN2; CPT1; CACT; CPT2; CPS1; ARG1; ASL; OTC; UGT1A1; FAH; COL7A1; COL17A1; MMP1; KRT5; LAMA3; LAMB3; LAMC2; ITGB4; and/or ATP7B), and the like. The above list of proteins refers to mammalian proteins, and in many embodiments human proteins, where the nucleotide and amino acid sequences of the above proteins are generally known to those of skill in the art.

Nonlimiting examples of targeted nucleases (genome-editing enzymes) include naturally occurring and recombinant nucleases, e.g. restriction endonucleases, meganucleases homing endonucleases, CRISPR/Cas effector proteins (e.g., CRISPR/Cas endonucleases such as Cas9, Cas12, Cas13, and the like). Any targeted nuclease(s) that are specific for the integration site of interest and promote the cleavage of an integration site may be encoded by a nucleotide sequence of interest. any examples of nucleases are known in the art, including Zinc finger nucleases (ZFNs), Transcription Activator-Like Effector Nucleases (TALENs), CRISPR/Cas effector proteins, meganucleases, homing endonucleases, restriction endonucleases, and the like (e.g., RecBCD endonuclease, T7 endonuclease, T4 endonuclease IV, Bal 31 endonuclease, Endonuclease I (endo I), Endonuclease II (endo VI, exo III), Micrococcal nuclease, Neurospora endonuclease, S1-nuclease, P1-nuclease, Mung bean nuclease I, Ustilago nuclease, Dnase I, AP endonuclease, EndoR, etc.).

In various embodiments, the disclosure provides variant AAV capsid polypeptides capable of forming capsids capable of packaging a variety of therapeutic molecules, including nucleic acids and polypeptides. In various embodiments, the disclosure provides for AAV vectors capable of containing nucleic acid inserts, including for example, transgene inserts or other nucleic acid inserts. This allows for vectors capable of expressing polypeptides. Such nucleic acids can comprise heterologous nucleic acid, nucleic acid gene products, and polypeptide gene products.

In some embodiments, the nucleotide sequence of interest encodes a non-coding RNA, encodes a protein coding sequence, is an expression cassette, is a multi-expression cassette, is a sequence for homologous recombination, is a genomic gene targeting cassette, and/or is a therapeutic expression cassette. In some embodiments, the expression cassette is a CRISPR/CAS expression system (e.g., including a CRISPR/Cas guide RNA and a CRISPR/Cas effector protein such as Cas9 or Cpf1. In some embodiments, a nucleic acid insert comprises a heterologous nucleic acid comprising a nucleotide sequence encoding a heterologous gene product, e.g., a nucleic acid gene product or a polypeptide gene product. As noted above, in some embodiments, the gene product is an interfering RNA (e.g., shRNA, siRNA, miRNA). In some embodiments, the gene product is an aptamer. The gene product can be a self-complementary nucleic acid. In some embodiments, the gene product is a polypeptide-coding RNA (e.g., an mRNA).

Suitable heterologous gene product includes interfering RNA, antisense RNA, ribozymes, and aptamers. Where the gene product is an interfering RNA (RNAi), suitable RNAi include RNAi that decrease the level of a target polypeptide in a cell.

In some embodiments, exemplary polypeptides include neuroprotective polypeptides and/or anti-angiogenic polypeptides (both of which are therapeutic polypeptides). Suitable polypeptides include, but are not limited to, glial derived neurotrophic factor (GDNF), fibroblast growth factor 2 (FGF-2), neurturin, ciliary neurotrophic factor (CNTF), nerve growth factor (NGF; e.g., nerve growth factor-.beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Flt-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic products encoded by the heterologous nucleic acid sequence include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGF.alpha., activins, inhibins, or any of the bone morphogenic proteins (BMP) BMPs 1-15, any one of the heregulin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful heterologous nucleic acid sequence products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present disclosure. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful heterologous nucleic acid sequence products include any one of the receptors for the hormones, growth factors, cytokines, lymphokines, regulatory proteins and immune system proteins. Useful heterologous nucleic acid sequences also include receptors for cholesterol regulation and/or lipid modulation, including the low density lipoprotein (LDL) receptor, high density lipoprotein (HDL) receptor, the very low density lipoprotein (VLDL) receptor, and scavenger receptors. The disclosure also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4 C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful heterologous nucleic acid sequence products include, carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, porphobilinogen deaminase, cystathionine beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence. Still other useful gene products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding .beta.-glucuronidase (GUSB)).

In some embodiments, useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, used to reduce overexpression of a target.

Host Cells and Packaging

Host cells are necessary for generating infectious AAV vectors as well as for generating AAV virions based on the disclosed AAV vectors. Accordingly, the present disclosure provides host cells for generation and packaging of AAV virions based on the AAV vectors of the present disclosure. A variety of host cells are known in the art and find use in the methods of the present disclosure. Any host cells described herein or known in the art can be employed with the compositions and methods described herein.

The present disclosure provides host cells, e.g., comprising a subject rAAV particle (virion) and/or a subject nucleic acid. A subject host cell can be an isolated cell, e.g., a cell in in vitro culture. In some cases, the cell is in vivo. A subject host cell can be useful for producing a subject AAV vector or AAV virion, as described below. Where a subject host cell is used to produce a subject AAV virion, it is referred to as a “packaging cell.” In some embodiments, a subject host cell is stably genetically modified with a subject AAV vector. In other embodiments, a subject host cell is transiently genetically modified with a subject AAV vector.

In some embodiments, a subject nucleic acid is introduced stably or transiently into a host cell, using established techniques, including, but not limited to, electroporation, calcium phosphate precipitation, liposome-mediated transfection, baculovirus infection, and the like. For stable transformation, a subject nucleic acid will generally further include a selectable marker, e.g., any of several well-known selectable markers such as neomycin resistance, and the like.

In some embodiments, the host cell for use in generating infectious virions can be selected from any biological organism, including prokaryotic (e.g., bacterial) cells, and eukaryotic cells, including, insect cells, yeast cells and mammalian cells. A subject host cell is generated by introducing a subject nucleic acid (i.e., AAV vector) into any of a variety of cells, e.g., mammalian cells, including, e.g., murine cells, and primate cells (e.g., human cells). Particularly desirable host cells are selected from among any mammalian species. In some embodiments, cells include without limitation, cells such as A549, WEHI, 10T1/2, BHK, MDCK, COS 1, COS 7, BSC 1, BSC 40, BMT 10, WI38, HeLa, CHO, 293, Vero, NIH 3T3, PC12, Huh-7 Saos, C2C12, RAT1, Sf9, L cells, HT1080, human embryonic kidney (HEK), human embryonic stem cells, human adult tissue stem cells, pluripotent stem cells, induced pluripotent stem cells, reprogrammed stem cells, organoid stem cells, bone marrow stem cells, HLHepG2, HepG2 and primary fibroblast, hepatocyte and myoblast cells derived from mammals including human, monkey, mouse, rat, rabbit, and hamster. The selection of the mammalian species providing the cells is not a limitation of this disclosure; nor is the type of mammalian cell, i.e., fibroblast, hepatocyte, tumor cell, etc. The requirement for the cell used is it is capable of infection or transfection by an AAV vector. In some embodiments, the host cell is one that has Rep and Cap stably transfected in the cell, including in some embodiments a variant AAV capsid polypeptide as described herein. In some embodiments, the host cell expresses a variant AAV capsid polypeptide of the disclosure or part of an AAV vector as described herein, such as a heterologous nucleic acid sequence contained within the AAV vector.

In some embodiments, the preparation of a host cell according to the disclosure involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods providing the desired nucleotide sequence.

In some embodiments, introduction of the AAV vector into the host cell may also be accomplished using techniques known to the skilled artisan and as discussed throughout the specification. In a preferred embodiment, standard transfection techniques are used, e.g., CaPO₄ transfection or electroporation, and/or infection by hybrid adenovirus/AAV vectors into cell lines such as the human embryonic kidney cell line HEK293 (a human kidney cell line containing functional adenovirus E1 genes providing trans-acting E1 proteins).

In some embodiments, a subject genetically modified host cell includes, in addition to a nucleic acid comprising a nucleotide sequence encoding a variant AAV capsid protein, as described above, a nucleic acid that comprises a nucleotide sequence encoding one or more AAV Rep proteins. In other embodiments, a subject host cell further comprises an AAV vector. An AAV virion can be generated using a subject host cell. Methods of generating an AAV virion are described in, e.g., U.S. Patent Publication No. 2005/0053922 and U.S. Patent Publication No. 2009/0202490.

In addition to an AAV vector, in some cases the host cell contains the sequences driving expression of the AAV capsid polypeptide (including variant AAV capsid polypeptides and non-variant parent capsid polypeptides) in the host cell and Rep sequences of the same serotype as the serotype of the AAV Inverted Terminal Repeats (ITRs) found in the nucleic acid insert (also referred to as a heterologous nucleotide sequence or the “nucleotide sequence of interest”), or a cross-complementing serotype. The AAV Cap and Rep sequences may be independently obtained from an AAV source and may be introduced into the host cell in any manner known to one of skill in the art or as described herein. Additionally, when pseudotyping an AAV vector in an AAV8 capsid for example, the sequences encoding each of the essential Rep proteins may be supplied by AAV8, or the sequences encoding the Rep proteins may be supplied by different AAV serotypes (e.g., AAV1, AAV2, AAV3, AAV4, AAVS, AAV6, AAV7, and/or AAV9).

In some embodiments, the host cell stably contains the capsid protein under the control of a suitable promoter (including, for example, the variant AAV capsid polypeptides of the disclosure), such as those described above. In some embodiments, the capsid protein is expressed under the control of an inducible promoter. In some embodiments, the capsid protein is supplied to the host cell in trans. When delivered to the host cell in trans, the capsid protein may be delivered via a plasmid containing the sequences necessary to direct expression of the selected capsid protein in the host cell. In some embodiments, when delivered to the host cell in trans, the vector encoding the capsid protein (including, for example, the variant AAV capsid polypeptides of the disclosure) also carries other sequences required for packaging the AAV, e.g., the Rep sequences.

In some embodiments, the host cell stably contains the Rep sequences under the control of a suitable promoter, such as those described above. In some embodiments, the essential Rep proteins are expressed under the control of an inducible promoter. In another embodiment, the Rep proteins are supplied to the host cell in trans. When delivered to the host cell in trans, the Rep proteins may be delivered via a plasmid containing the sequences necessary to direct expression of the selected Rep proteins in the host cell. In some embodiments, when delivered to the host cell in trans, the vector encoding the capsid protein (including, for example, the variant AAV capsid polypeptides of the disclosure) also carries other sequences required for packaging the AAV vector, e.g., the Rep sequences.

In some embodiments, the Rep and Cap sequences may be transfected into the host cell on a single nucleic acid molecule and exist stably in the cell as an unintegrated episome. In another embodiment, the Rep and Cap sequences are stably integrated into the chromosome of the cell. Another embodiment has the Rep and Cap sequences transiently expressed in the host cell. For example, a useful nucleic acid molecule for such transfection comprises, from 5′ to 3′, a promoter, an optional spacer interposed between the promoter and the start site of the Rep gene sequence, an AAV Rep gene sequence, and an AAV Cap gene sequence.

Although the molecule(s) providing Rep and capsid can exist in the host cell transiently (i.e., through transfection), in some embodiments, one or both of the Rep and capsid proteins and the promoter(s) controlling their expression be stably expressed in the host cell, e.g., as an episome or by integration into the chromosome of the host cell. The methods employed for constructing embodiments of the disclosure are conventional genetic engineering or recombinant engineering techniques such as those described in the references above.

In some embodiments, the packaging host cell can require helper functions in order to package the AAV vector of the disclosure into an AAV virion. In some embodiments, these functions may be supplied by a herpesvirus. In some embodiments, the necessary helper functions are each provided from a human or non-human primate adenovirus source, and are available from a variety of sources, including the American Type Culture Collection (ATCC), Manassas, Va. (US). In some embodiments, the host cell is provided with and/or contains an E1a gene product, an E1b gene product, an E2a gene product, and/or an E4 ORF6 gene product. In some embodiments, the host cell may contain other adenoviral genes such as VAI RNA. In some embodiments, no other adenovirus genes or gene functions are present in the host cell.

Methods for Generating an AAV Virion

In various embodiments, the disclosure provides a method for generating an AAV virion of the disclosure. A variety of methods for generating AAV virions are known in the art and can be used to generate AAV virions comprising the AAV vectors described herein. Generally, the methods involve inserting or transducing an AAV vector of the disclosure into a host cell capable of packaging the AAV vector into an AAV virion. Exemplary methods are described and referenced below; however, any method known to one of skill in the art can be employed to generate the AAV virions of the disclosure.

An AAV vector comprising a heterologous nucleic acid and used to generate an AAV virion can be constructed using methods that are well known in the art. See, e.g., Koerber et al. (2009) Mol. Ther., 17:2088; Koerber et al. (2008) Mol Ther., 16: 1703-1709; as well as U.S. Pat. Nos. 7,439,065, 6,951,758, and 6,491,907. For example, the heterologous sequence(s) can be directly inserted into an AAV genome with the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce AAV virions, an AAV vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome-mediated gene transfer (Mannino et al. (1988) BioTechniques 6:682-690), lipid-mediated transduction (Feigner et al. (1987) Proc. Natl. Acad. Sci. USA 84:7413-7417), and nucleic acid delivery using high-velocity microprojectiles (Klein et al. (1987) Nature 327:70-73).

Suitable host cells for producing AAV virions include any species and/or type of cell that can be, or have been, used as recipients of a heterologous AAV DNA molecule, and can support the expression of required AAV production cofactors from helper viruses. Such host cells can include but are not limited to microorganisms, yeast cells, insect cells, and mammalian cells, that can be, or have been, used as recipients of a heterologous DNA molecule. The term includes the progeny of the original cell transfected. Thus, a “host cell” as used herein generally refers to a cell transfected with an exogenous DNA sequence. Cells from the stable human cell line, HEK293 (readily available through, e.g., the American Type Culture Collection under Accession Number ATCC CRL1573) can be used. The human cell line HEK293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The HEK293 cell line is readily transfected, and provides a convenient platform in which to produce AAV virions.

Methods of producing an AAV virion in insect cells are known in the art, and can be used to produce a subject AAV virion. See, e.g., U.S. Patent Publication No. 2009/0203071; U.S. Pat. No. 7,271,002; and Chen (2008) Mol. Ther. 16:924.

In some embodiments, the AAV virion or AAV vector is packaged into an infectious virion or virus particle, by any of the methods described herein or known in the art.

In some embodiments, the variant AAV capsid polypeptide allows for similar packaging as compared to a non-variant parent capsid polypeptide. In some embodiments, an AAV vector packaged with the variant AAV capsid polypeptides transduce into cells in vivo better than a vector packaged from non-variant parent capsid polypeptides. In some embodiments, the AAV vector packaged with the variant AAV capsid polypeptides transduce into cells in vitro better than a vector packaged from non-variant parent capsid polypeptides. In some embodiments, the variant AAV capsid polypeptides result in nucleic acid expression higher than a nucleic acid packaged from non-variant parent capsid polypeptides. In some embodiments, the AAV vector packaged with said variant AAV capsid polypeptides result in transgene expression better than a transgene packaged from non-variant parent capsid polypeptides.

Pharmaceutical Compositions & Dosing

The present disclosure provides pharmaceutical compositions useful in treating subjects according to the methods of the disclosure as described herein. Further, the present disclosure provides dosing regimens for administering the described pharmaceutical compositions. The present disclosure provides pharmaceutical compositions comprising: a) a subject AAV vector or AAV virion, as described herein as well as therapeutic molecules packaged by or within capsids comprising variant polypeptides as described herein; and b) a pharmaceutically acceptable carrier, diluent, excipient, or buffer. In some embodiments, the pharmaceutically acceptable carrier, diluent, excipient, or buffer is suitable for use in a human.

Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro, (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7.sup.th ed., Lippincott, Williams, & Wilkins; and Handbook of Pharmaceutical Excipients (2000) A. H. Kibbe et al., eds., 3.sup.rd ed. Amer. Pharmaceutical Assoc.

A subject composition can comprise a liquid comprising a subject variant AAV capsid polypeptide of the disclosure or AAV virion comprising a variant AAV capsid polypeptide in solution, in suspension, or both. As used herein, liquid compositions include gels. In some cases, the liquid composition is aqueous. In some embodiments, the composition is an in situ gellable aqueous composition, e.g., an in situ gellable aqueous solution. Aqueous compositions have ophthalmically compatible pH and osmolality.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound. Preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Useful carriers include Vaseline.RTM., lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Pharmaceutical compositions and delivery systems appropriate for the AAV vector or AAV virion and methods and uses of are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

Doses can vary and depend upon whether the treatment is prophylactic or therapeutic, the type, onset, progression, severity, frequency, duration, or probability of the disease treatment is directed to, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

Methods and uses of the disclosure as disclosed herein can be practiced within about 1 hour to about 2 hours, about 2 hours to about 4 hours, about 4 hours to about 12 hours, about 12 hours to about 24 hours or about 24 hours to about 72 hours after a subject has been identified as having the disease targeted for treatment, has one or more symptoms of the disease, or has been screened and is identified as positive as set forth herein even though the subject does not have one or more symptoms of the disease. In some embodiments, the disclosure as disclosed herein can be practiced within about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 36 hours, about 48 hours, or about 72 hours or more. Of course, methods and uses of the disclosure can be practiced about 1 day to about 7 days, about 7 days to about 14 days, about 14 days to about 21 days, about 21 days to about 48 days or more, months or years after a subject has been identified as having the disease targeted for treatment, has one or more symptoms of the disease, or has been screened and is identified as positive as set forth herein. In some embodiments, the disclosure as disclosed herein can be practiced within about 1 day, about 2 days, about 3 days, about 4 days, about 5 days, about 6 days, about 7 days, about 8 days, about 9 days, about 10 days, about 11 days, about 12 days, about 14 days, about 21 days, about 36 days, or about 48 days or more.

In some embodiments, the present disclosure provides kits with packaging material and one or more components therein. A kit typically includes a label or packaging insert including a description of the components or instructions for use in vitro, in vivo, or ex vivo, of the components therein. A kit can contain a collection of such components, e.g., a variant AAV capsid polypeptide, an AAV vector, a nucleic acid encoding a variant AAV protein, and/or an AAV virion (in any combination thereof) and optionally a second active ingredient, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying the manufacturer, lot numbers, manufacturer location and date, expiration dates. Labels or inserts can include information identifying manufacturer information, lot numbers, manufacturer location and date. Labels or inserts can include information on a disease a kit component may be used for. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another incompatible treatment protocol or therapeutic regimen and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Method of Treating a Disease

The present disclosure provides methods for delivering a payload of interest to the central nervous system of an individual (e.g., methods of treating a disease in a subject by administering the AAV vectors and/or nucleic acids of the present disclosure), where AAV virus, vectors and/or nucleic acids described herein comprising one or more variant AAV capsid polypeptides of the present disclosure are administered to the individual. In an example embodiment, the disclosure provides a method of administering a pharmaceutical composition of the disclosure to a subject in need thereof to treat a disease of a subject. In various embodiments, the subject is not otherwise in need of administration of a composition of the disclosure.

In some embodiments, the variant AAV capsid polypeptides package a therapeutic expression cassette comprised of a heterologous nucleic acid comprising a nucleotide sequence encoding a heterologous gene product, such as for example a therapeutic protein. In some embodiments, the AAV virion or AAV vector comprises a therapeutic expression cassette comprised of a heterologous nucleic acid comprising a nucleotide sequence encoding a heterologous gene product, such as for example a therapeutic protein.

In some embodiments, the variant AAV capsid polypeptides of the disclosure are employed as part of vaccine delivery. Vaccine delivery can include delivery of any of the therapeutic proteins as well as nucleic acids described herein. In some embodiments, variant AAV capsid polypeptides of the disclosure are employed as part of a vaccine regimen and dosed according to the methods described herein.

In some embodiments, the variant AAV capsid polypeptides, the AAV virions, or AAV vectors of the disclosure are used in a therapeutic treatment regimen.

In some embodiments, the variant AAV capsid polypeptides, the AAV virions, or AAV vectors of the disclosure are used for therapeutic polypeptide production.

In some cases, a subject variant AAV capsid polypeptides or AAV vector, when introduced into the cells of a subject, provides for high level production of the heterologous gene product packaged by the variant AAV capsid polypeptides or encoded by the AAV vector. For example, a heterologous polypeptide packaged by the variant AAV capsid polypeptides or encoded by the AAV can be produced.

In some cases, subject variant AAV capsid polypeptides, AAV virion, or AAV vector, when introduced into a subject, provide for production of the heterologous gene product packaged by the variant AAV capsid polypeptides or encoded by the AAV vector in at least about 10%, at least about 15%, at least about 20%, at least about 25%, at least about 30%, at least about 35%, at least about 40%, at least about 50% at least about 60%, at least about 70%, at least about 80%, or more than 80%, of the target cells.

In some embodiments, the present disclosure provides a method of treating a disease, the method comprising administering to an individual in need thereof an effective amount of a therapeutic molecule packaged by the variant AAV capsid polypeptides or subject AAV vector as described above.

Subject variant AAV capsid polypeptides or subject AAV vectors can be administered systemically, regionally or locally, or by any route, for example, by injection, infusion, orally (e.g., ingestion or inhalation), or topically (e.g., transdermally). Possible delivery and administration methods can include parenteral, intravenous, intramuscular, intraperitoneal, intradermal, subcutaneous, intracavity, intracranial, transdermal (topical), transmucosal and rectal administration. Example administration and delivery routes include intravenous, intraperitoneal, intrarterial, parenteral, subcutaneous, intra-pleural, topical, dermal, intradermal, transdermal, transmucosal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic. In some cases the delivery route is systemic (e.g., parenteral, intravenous).

In some cases, a therapeutically effective amount of a therapeutic molecule packaged by the variant AAV capsid polypeptides or a subject AAV vectors is an amount that, when administered to an individual in one or more doses, is effective to slow the progression of the disease or disorder in the individual, or is effective to ameliorate symptoms. For example, a therapeutically effective amount of a therapeutic molecule packaged by the variant AAV capsid polypeptides or a subject AAV vectors can be an amount that, when administered to an individual in one or more doses, is effective to slow the progression of the disease by at least about 15%, at least about 20%, at least about 25%, at least about 30%, at least about 40%, at least about 50%, at least about 60%, at least about 70%, at least about 80%, or more than about 80%, compared to the progression of the disease in the absence of treatment with the therapeutic molecule packaged by the variant AAV capsid polypeptides or AAV vectors.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, or complication of a disease. Thus, a satisfactory clinical endpoint is achieved when there is an incremental improvement or a partial reduction in an adverse symptom, disorder, illness, or complication caused by or associated with a disease, or an inhibition, decrease, reduction, suppression, prevention, limit or control of worsening or progression of one or more adverse symptoms, disorders, illnesses, or complications caused by or associated with the disease, over a short or long duration (hours, days, weeks, months, etc.).

Improvement of clinical symptoms can also be monitored by one or more methods known to the art, and used as an indication of therapeutic effectiveness. Clinical symptoms may also be monitored by anatomical or physiological means, such as indirect ophthalmoscopy, fundus photography, fluorescein angiopathy, optical coherence tomography, electroretinography (full-field, multifocal, or other), external eye examination, slit lamp biomicroscopy, applanation tonometry, pachymetry, autorefraction, or other measures of functional vision. In some embodiments, a therapeutic molecule (including, for example, nucleic acid that includes a nucleotide sequence of interest) packaged by the variant AAV capsid polypeptides, a subject AAV vector, or AAV virus, when introduced into a subject, provides for production of a heterologous gene product (e.g., non-coding or coding RNA, a protein) for a period of time from about 2 days to about 6 months, e.g., from about 2 days to about 7 days, from about 1 week to about 4 weeks, from about 1 month to about 2 months, or from about 2 months to about 6 months. In some embodiments, therapeutic molecules packaged by the variant AAV capsid polypeptides, a subject AAV vector or virus, when introduced into a subject provides for production of the heterologous gene product for a period of time of more than 6 months, e.g., from about 6 months to 20 years or more, or greater than 1 year, e.g., from about 6 months to about 1 year, from about 1 year to about 2 years, from about 2 years to about 5 years, from about 5 years to about 10 years, from about 10 years to about 15 years, from about 15 years to about 20 years, or more than 20 years.

Multiple doses of a subject AAV virion can be administered to an individual in need thereof. Where multiple doses are administered over a period of time, an active agent is administered once a month to about once a year, from about once a year to once every 2 years, from about once every 2 years to once every 5 years, or from about once every 5 years to about once every 10 years, over a period of time. For example, a subject AAV virion is administered over a period of from about 3 months to about 2 years, from about 2 years to about 5 years, from about 5 years to about 10 years, from about 10 years to about 20 years, or more than 20 years. The actual frequency of administration, and the actual duration of treatment, depends on various factors. In some embodiments, the administration regimen is part of a vaccination regimen.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (e.g., RNA or protein), and the stability of the expressed molecule. One skilled in the art can readily determine a virion dose range to treat a patient having a particular disease or disorder based on the aforementioned factors, as well as other factors. Generally, doses will range from at least about, or more, for example, 1×10⁹, 1×10¹⁰, 1×10¹¹, 1×10¹², 1×10¹³, or 1×10¹⁴, or more, vector genomes per kilogram (vg/kg) of the weight of the subject, to achieve a therapeutic effect.

An effective amount or a sufficient amount can, but need not be, provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol.

An effective amount or a sufficient amount need not be effective in each and every subject treated, or a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use. Thus, appropriate amounts will depend upon the condition treated, the therapeutic effect desired, as well as the individual subject (e.g., the bioavailability within the subject, gender, age, etc.).

With regard to a disease or symptom thereof, or an underlying cellular response, a detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

Thus, a successful treatment outcome can lead to a “therapeutic effect,” or “benefit” of decreasing, reducing, inhibiting, suppressing, limiting, controlling or preventing the occurrence, frequency, severity, progression, or duration of a disease, or one or more adverse symptoms or underlying causes or consequences of the disease in a subject. Treatment methods and uses affecting one or more underlying causes of the disease or adverse symptoms are therefore considered to be beneficial. A decrease or reduction in worsening, such as stabilizing the disease, or an adverse symptom thereof, is also a successful treatment outcome.

A therapeutic benefit or improvement therefore need not be complete ablation of the disease, or any one, most or all adverse symptoms, complications, consequences or underlying causes associated with the disease. Thus, a satisfactory endpoint is achieved when there is an incremental improvement in a subject's disease, or a partial decrease, reduction, inhibition, suppression, limit, control or prevention in the occurrence, frequency, severity, progression, or duration, or inhibition or reversal, of the disease (e.g., stabilizing one or more symptoms or complications), over a short or long duration of time (hours, days, weeks, months, etc.). Effectiveness of a method or use, such as a treatment that provides a potential therapeutic benefit or improvement of a disease, can be ascertained by various methods.

Disclosed methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents and drugs. Such biologics (proteins), agents, drugs, treatments and therapies can be administered or performed prior to, substantially contemporaneously with or following any other method or use of the disclosure.

The compound, agent, drug, treatment or other therapeutic regimen or protocol can be administered as a combination composition, or administered separately, such as concurrently or in series or sequentially (prior to or following) delivery or administration of an AAV vector or AAV virion as described herein. The disclosure therefore provides combinations where a method or use of the disclosure is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of an AAV vector or AAV virion as described herein, to a subject. Specific non-limiting examples of combination embodiments therefore include the foregoing or other compound, agent, drug, therapeutic regimen, treatment protocol, process, remedy or composition.

Methods and uses of the disclosure also include, among other things, methods and uses that result in a reduced need or use of another compound, agent, drug, therapeutic regimen, treatment protocol, process, or remedy. For example, for a blood clotting disease, a method or use of the disclosure has a therapeutic benefit if in a given subject a less frequent or reduced dose or elimination of administration of a recombinant clotting factor protein to supplement for the deficient or defective (abnormal or mutant) endogenous clotting factor in the subject. Thus, in accordance with the disclosure, methods and uses of reducing need or use of another treatment or therapy are provided.

The disclosure is useful in animals including veterinary medical applications. Suitable subjects therefore include mammals, such as humans, as well as non-human mammals such as non-human primates. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases and others known to those of skill in the art.

In some embodiments, a method or use of the disclosure includes: (a) providing an AAV virion whose capsid comprises a variant AAV capsid polypeptide (e.g., prepared as described herein), wherein the AAV virion comprises a heterologous nucleic acid sequence (e.g., in some cases operably linked to an expression control element conferring transcription of said nucleic acid sequence); and (b) administering an amount of the AAV virion to the mammal such that said heterologous nucleic acid is expressed in the mammal.

In some embodiments, a method or use of the disclosure includes: (a) providing a therapeutic molecule packaged by variant AAV capsid polypeptides (e.g., prepared as described herein), wherein the therapeutic molecule comprises a heterologous nucleic acid sequence (e.g., which can in some cases be operably linked to an expression control element conferring transcription of said nucleic acid sequence); and (b) administering an amount of the therapeutic molecule (including, for example, a vaccine) packaged by variant AAV capsid polypeptides to the mammal such that said heterologous nucleic acid is expressed in the mammal.

In some embodiments, a method or use of the disclosure includes delivering or transferring a heterologous polynucleotide sequence into a mammal or a cell of a mammal, by administering a heterologous polynucleotide packaged by the variant AAV capsid polypeptides, a plurality of heterologous polynucleotides packaged by variant AAV capsid polypeptides, an AAV virion prepared as described herein, or a plurality of AAV virions comprising the heterologous nucleic acid sequence to a mammal or a cell of a mammal, thereby delivering or transferring the heterologous polynucleotide sequence into the mammal or cell of the mammal. In some embodiments, the heterologous nucleic acid sequence encodes a protein expressed in the mammal, or where the heterologous nucleic acid sequence encodes an inhibitory sequence or protein that reduces expression of an endogenous protein in the mammal.

In some embodiments, a method or use of the disclosure includes is a method of delivering a payload of interest to the central nervous system of an individual, and includes administering to the individual a nucleic acid or a recombinant AAV (rAAV) particle as described herein (e.g., where the nucleic acid is a viral vector that encodes a variant AAV capsid protein and includes a nucleotides sequence of interest, where the rAAV particle comprises a variant AAV particle and a payload nucleic acid that includes a nucleotides sequence of interest).

### VI. EXAMPLE NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A variant adeno-associated virus (AAV) capsid protein that provides a viral particle with the ability to traverse the human blood brain barrier (BBB) and transduce cells of the CNS, wherein the variant AAV capsid protein comprises an amino acid sequence having 97% or more sequence identity with the amino acid sequence set forth in any one of SEQ ID NOs: 1-27.
2. The variant AAV capsid protein of aspect 1, wherein the variant AAV capsid protein comprises an amino acid sequence having 97% or more sequence identity with the amino acid sequence set forth in any one of SEQ ID NOs: 1-14.
3. The variant AAV capsid protein of aspect 1, wherein the variant AAV capsid protein comprises an amino acid sequence having 97% or more sequence identity with the amino acid sequence set forth in any one of SEQ ID NOs: 1-6.
4. The variant AAV capsid protein of aspect 1, wherein the variant AAV capsid protein comprises an amino acid sequence having 97% or more sequence identity with the amino acid sequence set forth in any one of SEQ ID NOs: 7-14.
5. The variant AAV capsid protein of aspect 1, wherein the variant AAV capsid protein comprises an amino acid sequence having 97% or more sequence identity with the amino acid sequence set forth in any one of SEQ ID NOs: 8-14.
6. The variant AAV capsid protein of aspect 1, wherein the variant AAV capsid protein comprises an amino acid sequence having 97% or more sequence identity with the amino acid sequence set forth in any one of SEQ ID NOs: 7 and 9-14.
7. The variant AAV capsid protein of aspect 1, wherein the variant AAV capsid protein comprises an amino acid sequence having 97% or more sequence identity with the amino acid sequence set forth in any one of SEQ ID NOs: 9-14.
8. The variant AAV capsid protein of aspect 1, wherein the variant AAV capsid protein comprises an amino acid sequence having 97% or more sequence identity with the amino acid sequence set forth in any one of SEQ ID NOs: 3, 4, and 11.
9. The variant AAV capsid protein of aspect 1, wherein the variant AAV capsid protein comprises an amino acid sequence having 97% or more sequence identity with the amino acid sequence set forth in any one of SEQ ID NOs: 15-27.
10. A variant adeno-associated virus (AAV) capsid protein that provides a viral particle with the ability to traverse the human blood brain barrier (BBB) and transduce cells of the CNS, wherein the variant AAV capsid protein comprises an amino acid sequence having 95% or more sequence identity with the amino acid sequence set forth in any one of SEQ ID NOs: SEQ ID NOs: 1-4, 6-10, 12-24, and 27.
11. The variant AAV capsid protein of aspect 10, wherein the variant AAV capsid protein comprises an amino acid sequence having 95% or more sequence identity with the amino acid sequence set forth in any one of SEQ ID NOs: 8-9 and 12-14.
12. The variant AAV capsid protein of aspect 10, wherein the variant AAV capsid protein comprises an amino acid sequence having 95% or more sequence identity with the amino acid sequence set forth in any one of SEQ ID NOs: 9 and 12-14.
13. The variant AAV capsid protein of aspect 10, wherein the variant AAV capsid protein comprises an amino acid sequence having 95% or more sequence identity with the amino acid sequence set forth in any one of SEQ ID NOs: 15-24, and 27.
14. A nucleic acid comprising a nucleotide sequence that encodes the variant AAV capsid protein of any one of aspects 1-13.
15. The nucleic acid of aspect 14, further comprising a nucleotide sequence of interest flanked by inverted terminal repeat sequences (ITRs).
16. The nucleic acid of aspect 15, wherein the nucleotide sequence of interest encodes a polypeptide.
17. The nucleic acid of aspect 15, wherein the nucleotide sequence of interest encodes a non-coding RNA.
18. The nucleic acid of any one of aspects 14-17, wherein the nucleic acid is an AAV vector.
19. A cell comprising the nucleic acid of any one of aspects 14-18.
20. A recombinant AAV particle comprising:
   (a) the variant AAV capsid protein of any one of aspects 1-13; and
   (b) a nucleic acid payload of interest.
21. The recombinant AAV particle of aspect 20, wherein the nucleic acid payload of interest encodes a polypeptide.
22. The recombinant AAV particle of aspect 21, wherein the polypeptide is a genome-editing enzyme.
23. The recombinant AAV particle of aspect 22, wherein the genome-editing enzyme is a CRISPR/Cas effector protein, a zinc finger nuclease, or a TALEN.
24. The recombinant AAV particle of aspect 21, wherein the polypeptide is a therapeutic protein.
25. The recombinant AAV particle of aspect 20, wherein the nucleic acid payload of interest is a non-coding RNA or encodes said non-coding RNA.
26. The recombinant AAV particle of aspect 25, wherein the non-coding RNA is a short hairpin RNA (shRNA) or an aptamer.
27. The recombinant AAV particle of aspect 25, wherein the non-coding RNA is a CRISPR/Cas guide RNA.
28. A cell comprising the recombinant AAV particle of any one of aspects 20-27.
29. A method of delivering a payload of interest to the central nervous system of an individual, the method comprising administering to the individual the nucleic acid of 18 or the recombinant AAV particle of any one of aspects 19-27.
30. The method of aspect 29, wherein said administering comprises parenteral administration of the recombinant AAV particle.

The following examples are offered by way of illustration and not by way of limitation.

### VII. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

In the studies disclosed below, an in vitro (human) model of the blood brain barrier (BBB) grown in transwells was used to screen a shuffled AAV capsid library to select for rAAVs that cross the blood brain barrier and transduce astrocytes and/or neurons. A number of rAAVs were generated that exhibit robust ability to cross the BBB. Both neurons and astrocytes can be targeted/transduced using rAAVs that were generated.

### Example 1: Developing an Assay to Screen for Novel Adeno-Associated Virus Vectors for the Delivery of Gene Therapy to the Central Nervous System

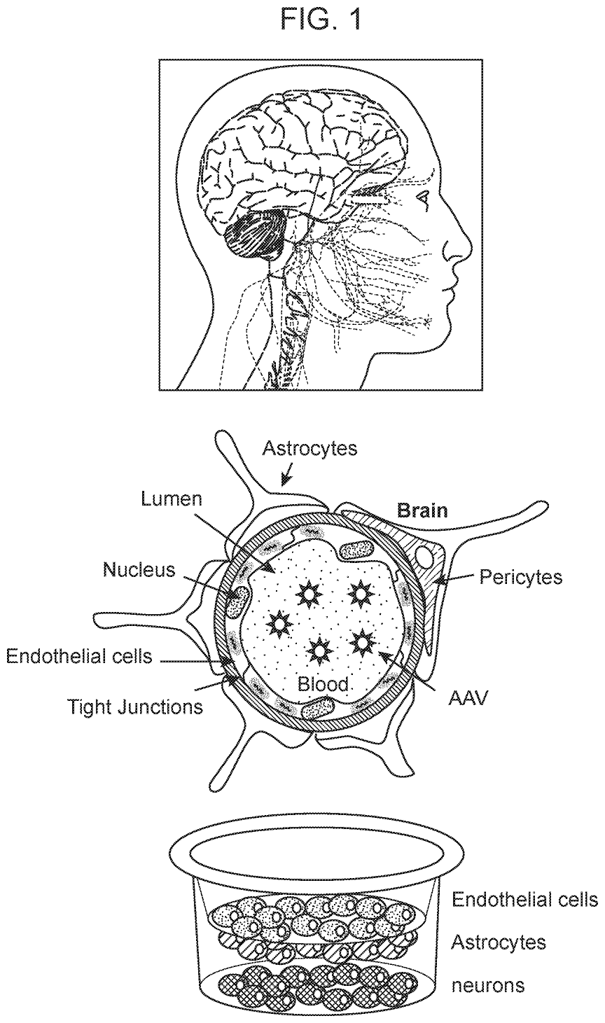

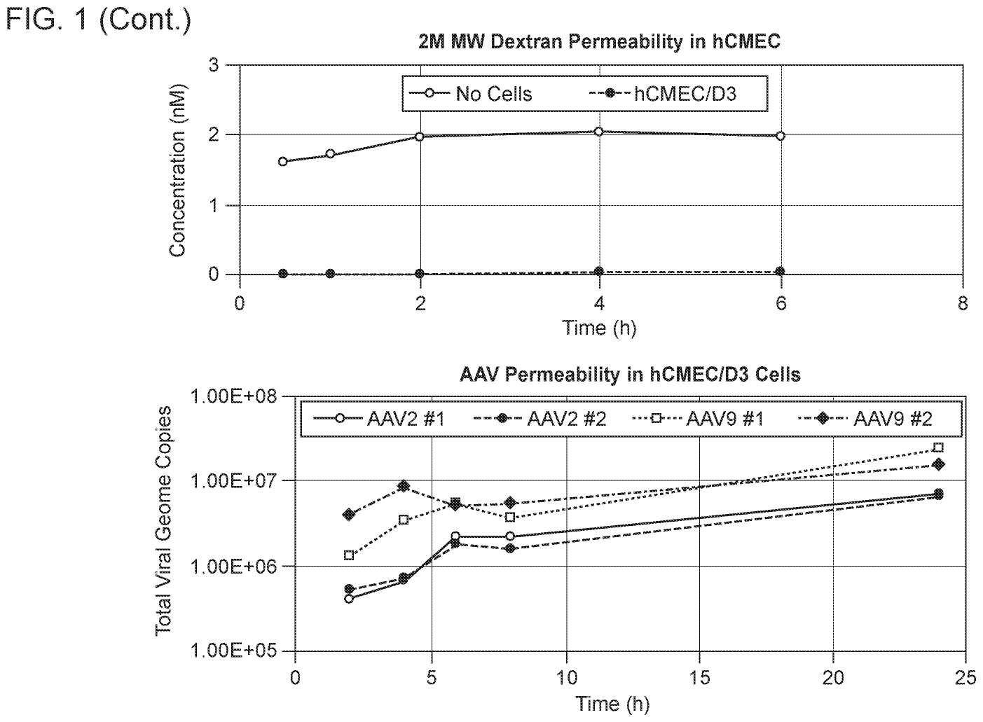

To overcome the species and cell-type limits of the AAV vectors, AAV vectors were selected from a shuffled library using a human model of the BBB that has previously been used to study BBB structure and function. The BBB consists of endothelial cells that form the walls of capillaries in the brain, and astrocytes and pericytes that directly associate with the endothelial cells (FIG. 1, top middle). To mimic the human BBB in culture, a well-studied transwell system (FIG. 1, top right) was used: a confluent layer of human cerebral endothelial cell line (hCMEC/D3) was cultured on the top of a transwell membrane, while a layer of human primary astrocytes was cultured on the bottom of the membrane. Furthermore, a layer of target cells (neurons) was cultured on the bottom of the dish that holds the transwell.

The confluent mono-layer of hCMEC/D3 cells formed tight junctions which prevented the diffusion of dextran molecules of 2,000,000 MW (approximately half the size of AAV) across the transwell (FIG. 1, bottom left). The transwell BBB model allowed a higher amount of AAV9 (which has been previously reported to cross BBB more efficiently than other natural AAV serotypes) to cross than AAV2 (FIG. 1, bottom right).

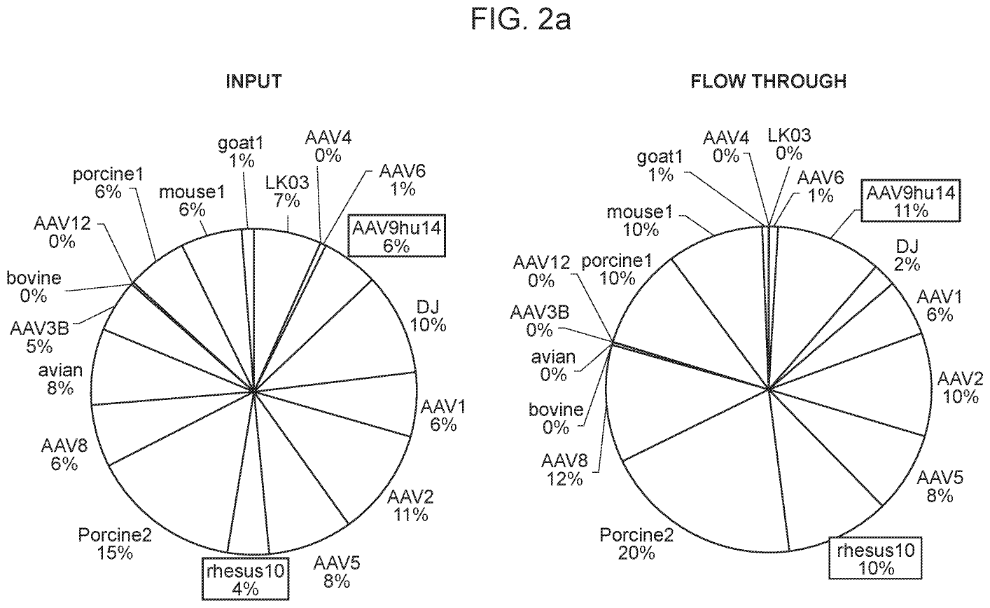

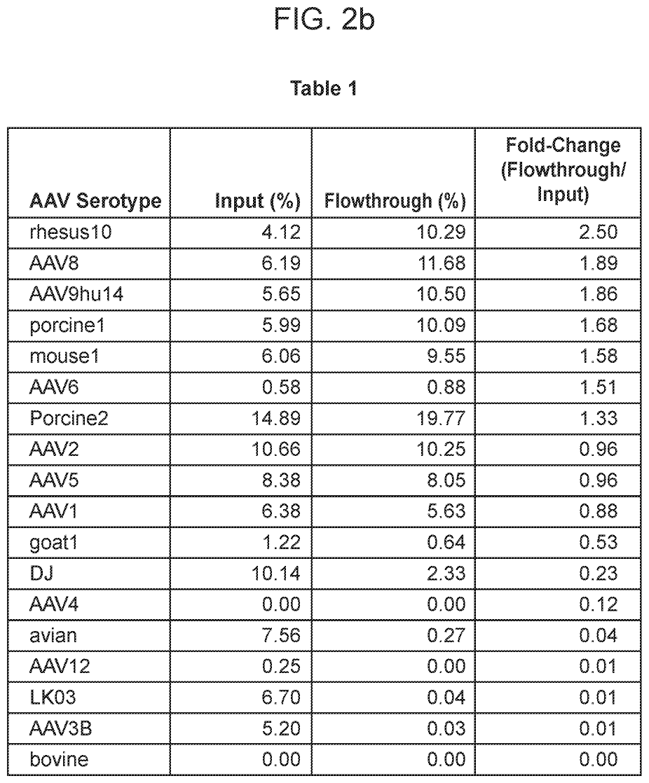

A mock selection in the human BBB transwell model was performed using 18 naturally isolated AAV serotypes, including AAV9 and AAV.rhesus10, which were known to have high efficiency in crossing the BBB. All viruses were barcoded for easy identification using high throughput sequencing. The virus composition in the input and flowthrough of the transwell was compared. AAV.rhesus10 and AAV9 were at top 1 and 3 of the viruses whose proportion increased in the flowthrough compared to input, 2.5- and 1.86-fold, respectively (see FIGS. 2a and 2b). While flowthough proportions of AAV2 and AAV-DJ, which are known to cross the BBB less efficiently, decreased by 0.96- and 0.23-fold in comparison to the input. These data indicated that the transwell BBB system was indeed useful for selecting AAV vectors that cross the BBB efficiently.

### Example 2: Identifying Novel Adeno-Associated Virus Vectors for the Delivery of Gene Therapy to the Central Nervous System

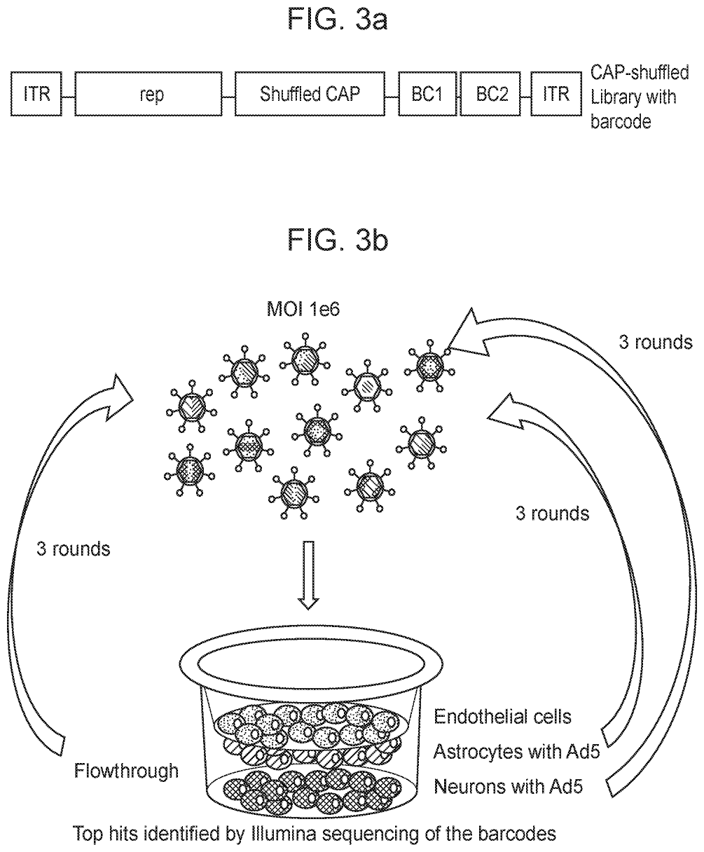

A selection of the barcoded and capsid-shuffled AAV library was performed in the human BBB transwell model for viruses that enter selectively cross the BBB and enter the astrocytes or neurons (FIG. 3). A barcoded capsid-shuffled-AAV library was placed on the top of the transwell and the viruses were allowed to pass through the endothelial cells and supporting cells. AAVs that traversed the BBB were isolated for several more rounds of selection. The barcodes of the top AAVs were identified by Illumina sequencing. PacBio sequencing was also performed to acquire large scale capsid sequences that facilitated prediction of the regions of the viruses required for crossing the BBB and/or transduce astrocytes and neurons.

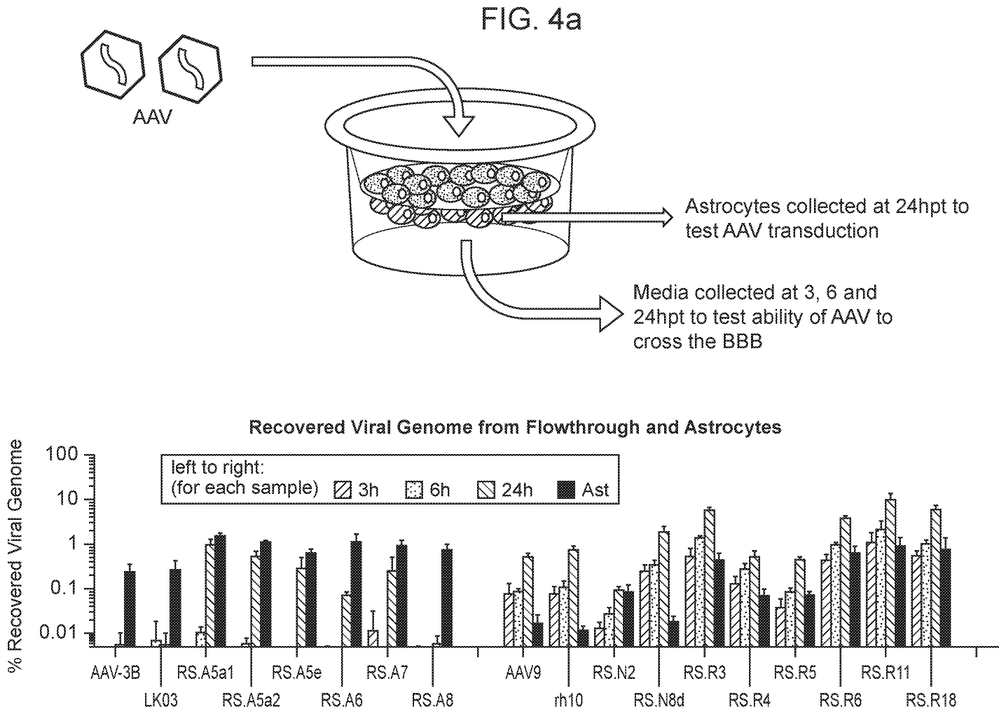

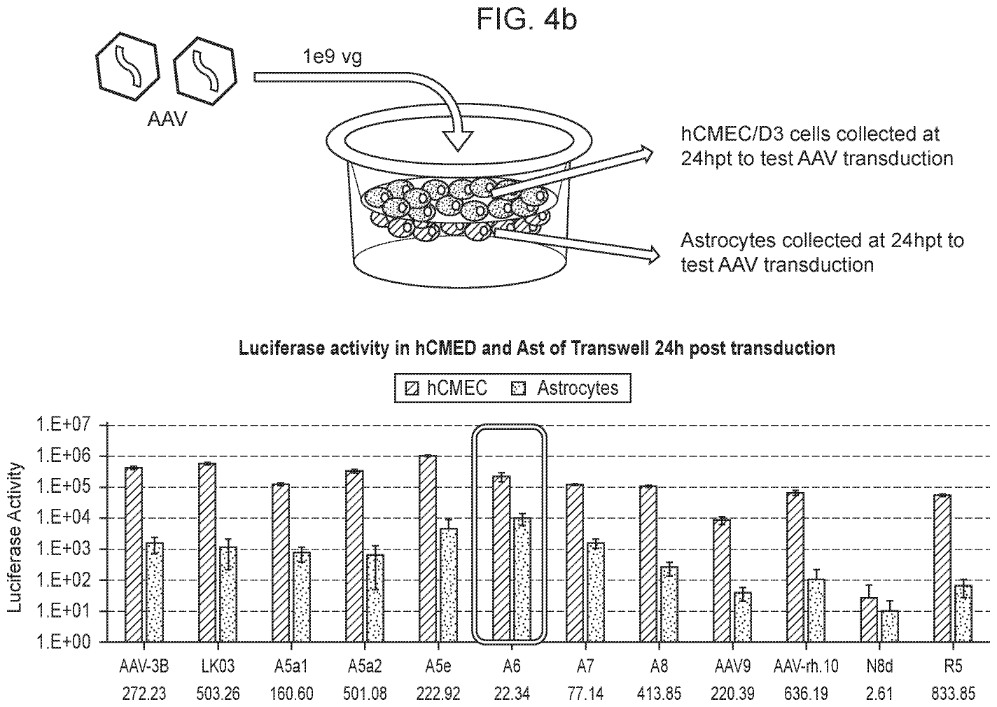

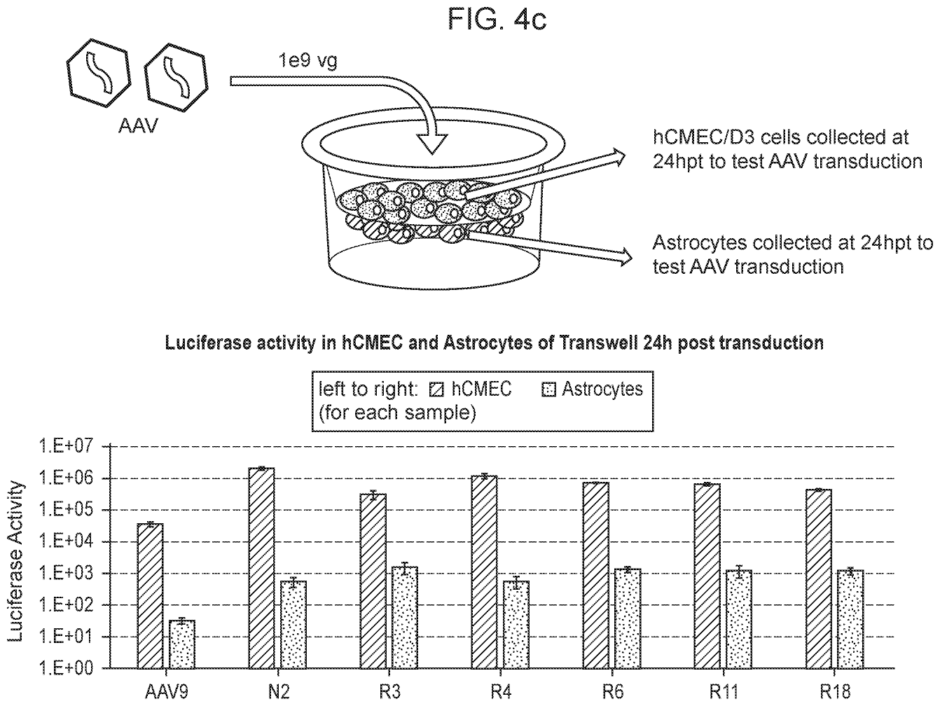

14 AAV capsid sequences were vectorized (6 for astrocytes and 8 for neurons) and their ability to cross the BBB and transduce endothelial cells and astrocytes was tested in the transwell BBB model (FIG. 4). All AAV vectors selected from astrocytes (RS.A5a1, RS.A5a2, RS.A5e, RS.A6, RS.A7 and RS.A8) transduced astrocytes at higher efficiency than controls AAV3B and LK03 (FIG. 4). The AAVs selected from neurons were compared to AAV9 and AAV.rhesus10: 5 of them (RS.N8d, RS.R3, RS.R6, RS.R11 and RS.R18) showed higher efficiency in crossing the BBB, 2 of them (RS.R4 and RS.R5) showed similar efficiency to the controls, and RS.N2 was the only one that was less efficient than the controls, although it was still more efficient than the AAV selected for astrocytes.

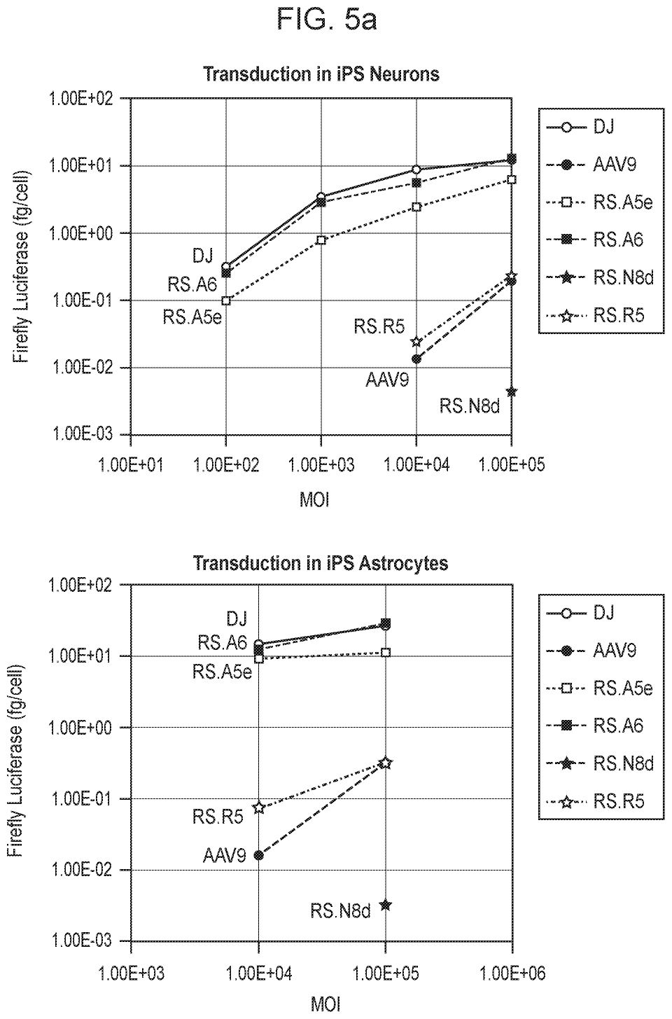

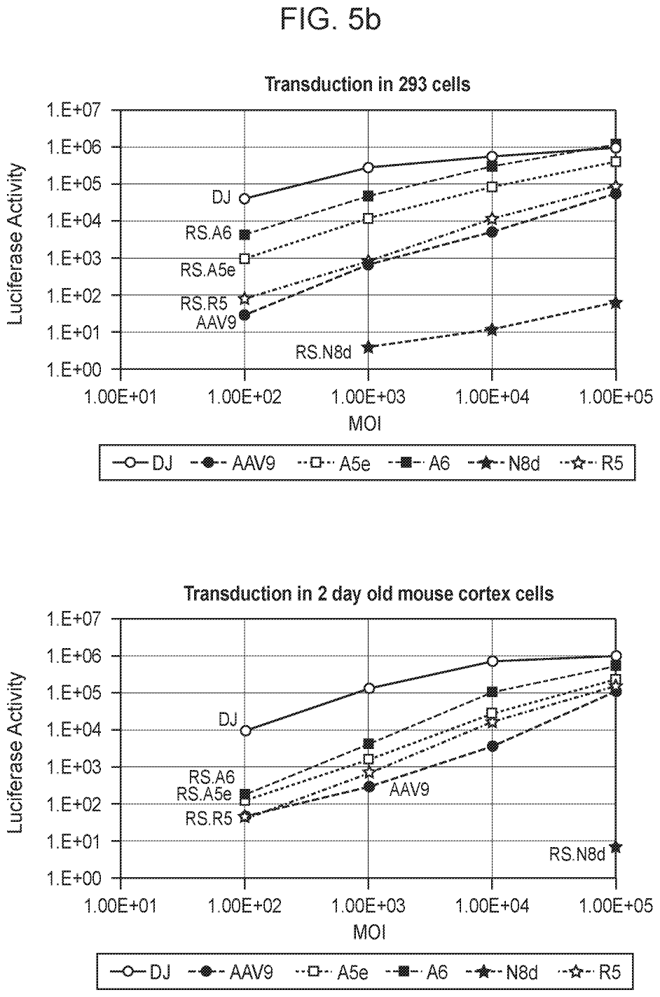

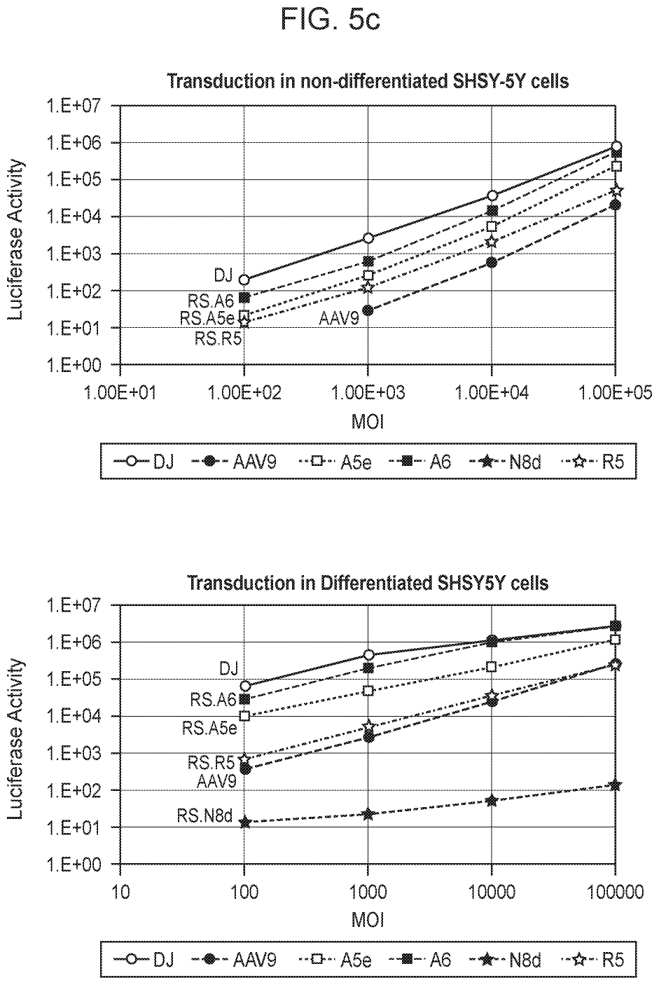

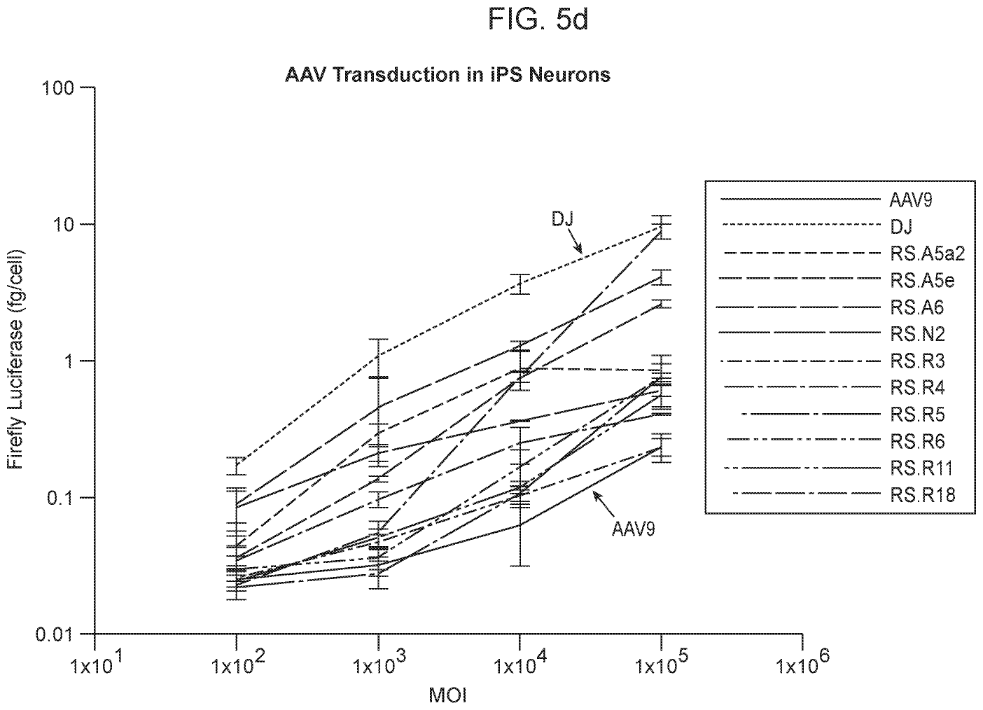

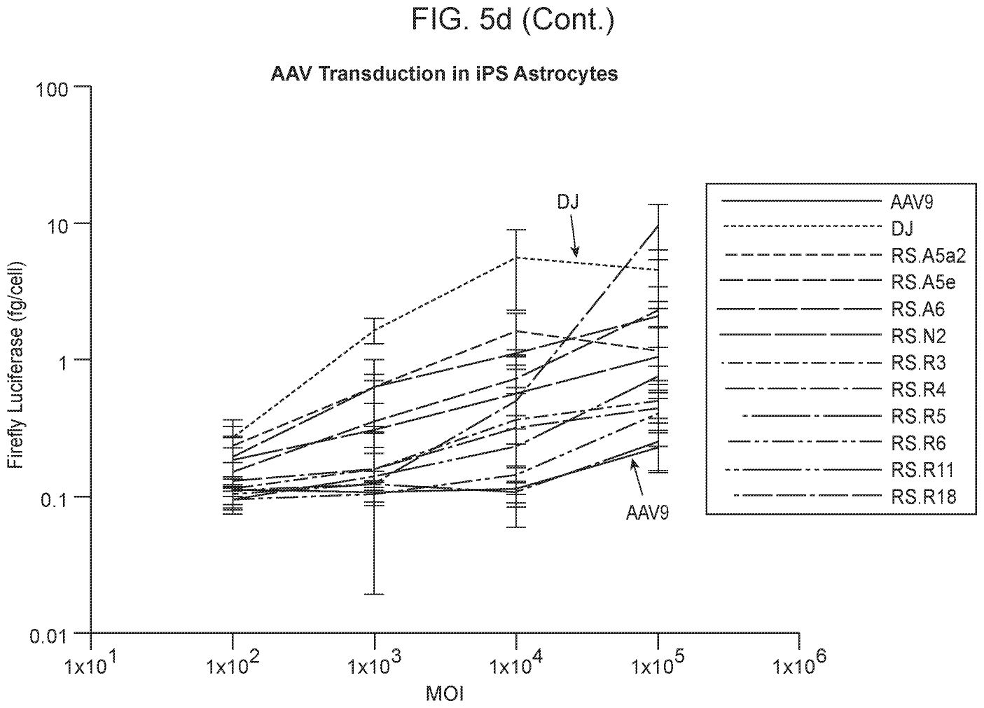

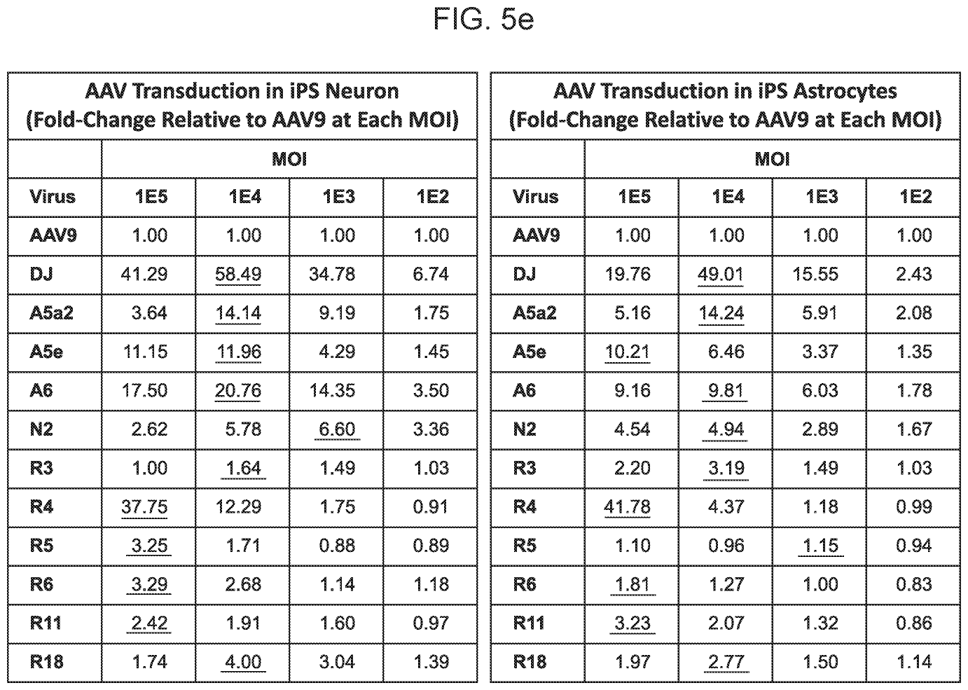

Transduction efficiency of selected rAAVs was also tested in iPS derived neurons and astrocytes, as well as 293 cells, 2 day old mouse cortex cells, and non-differentiated and differentiated SHSY5Y cells (FIG. 5). All of the AAVs except for RS.N8d transduced at higher or similar efficiency as AAV9 at low or high multiplicity of infection (MOI).

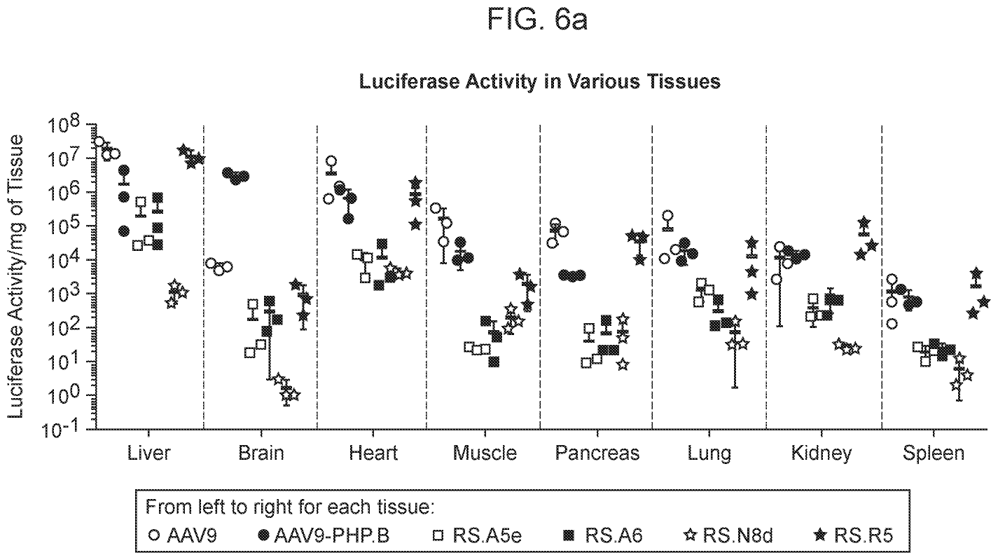

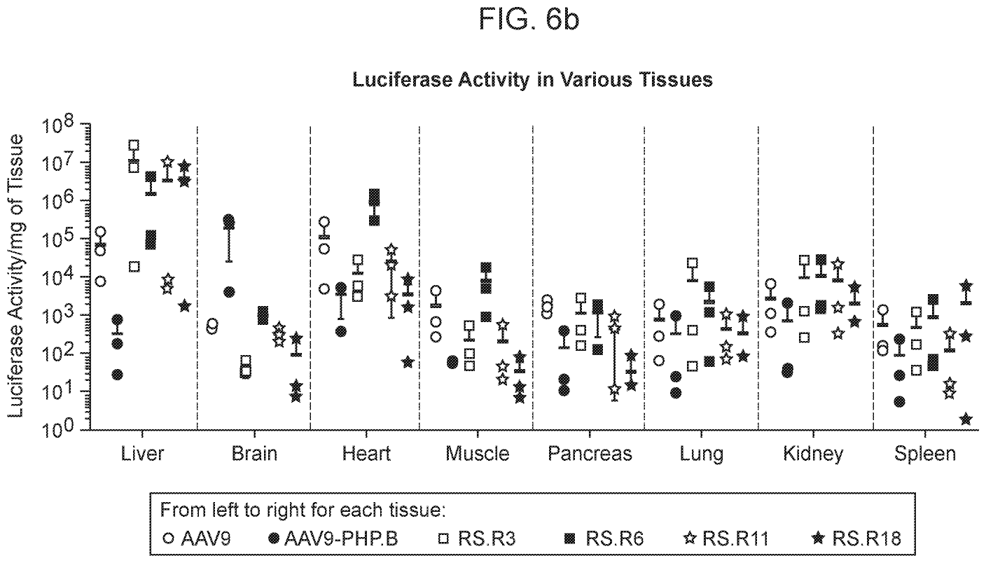

Viruses RS.A5e, RS.A6, RS.N8d, RS.R5, RS.R3, RS.R6, RS.R11 and RS.R18 as well as viruses A5e, A6, N8d and R5 were tested for transduction efficiency in vivo in mice 21 days or 30 days after retro-orbital injection (FIG. 6). The results showed that only RS.R6 transduced mouse brain as efficiently as AAV9 control, all others were less efficient. All variants were also less efficient than AAV9-PHP.B, one of the best currently known in C57BL/6 mice. However, this was not surprising because the viruses were selected using a model of the human system—and it was likely that they would not be very efficient in mice. This is similar to AAV9-PHP.B, which was selected in C57BL/6 mice, and is only efficient in C57BL/6 mice and not in other mouse strains or in non-human primates.

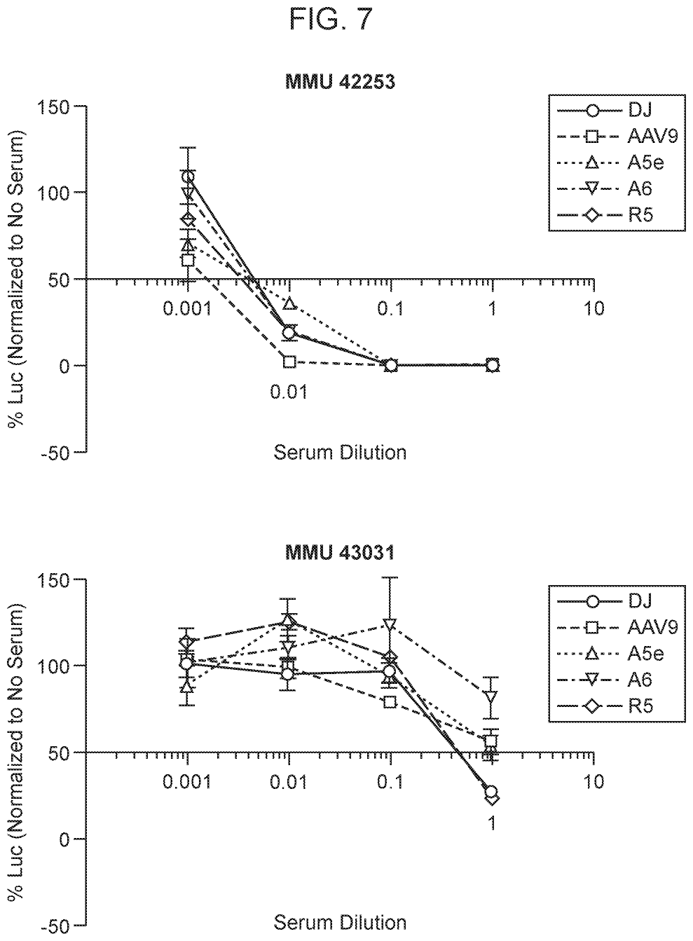

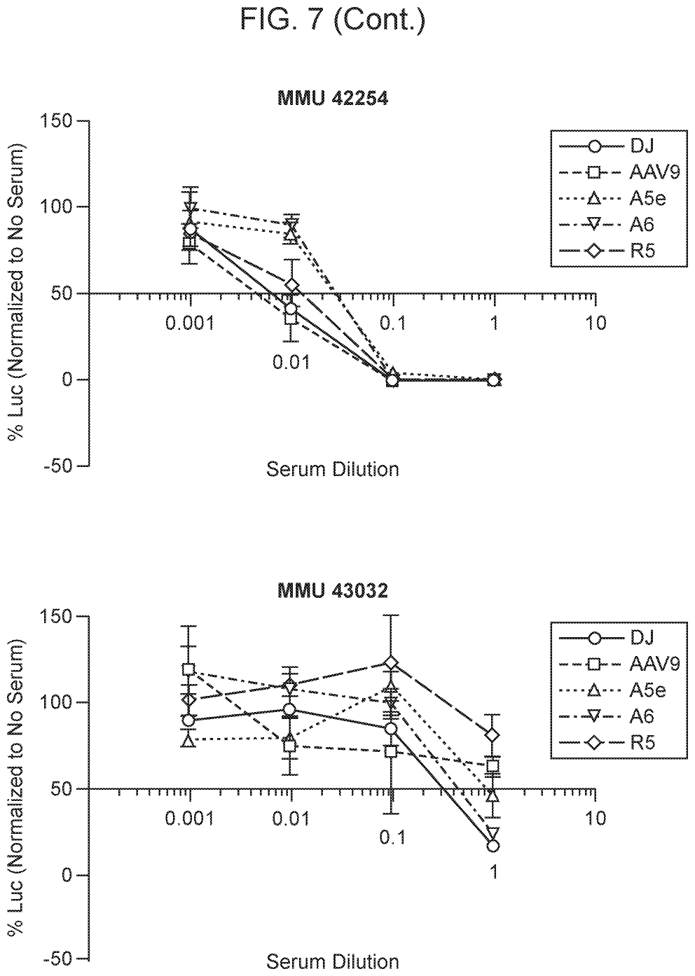

Non-human primate antibody neutralization assays were performed to test which non-human primate (NHP) will be promising/appropriate for pre-clinical NHP virus testing (e.g., won't mount a significant immune response against the introduced virus) (FIG. 7). The ability for antibodies in NHP serum to neutralize viruses identified in the screens discussed above was tested. The results indicated that in some NHP serum, all viruses (including AAV-DJ and AAV9 controls) were neutralized, and in others none of the viruses were neutralized. Note: these data do not speak to the transduction efficiency, but instead show that in at least some NHP serums the viruses were not neutralized by existing antibodies.

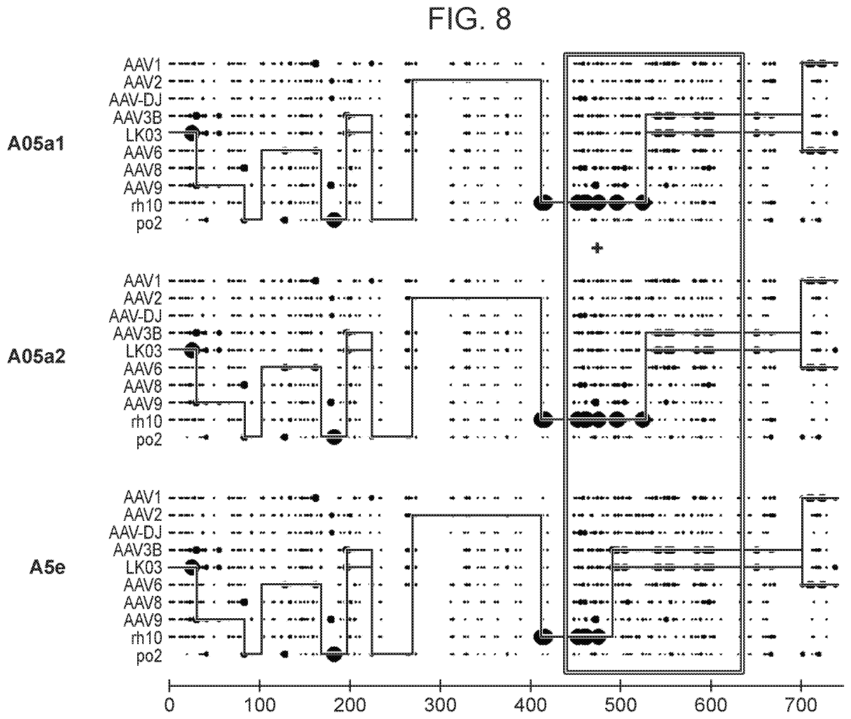

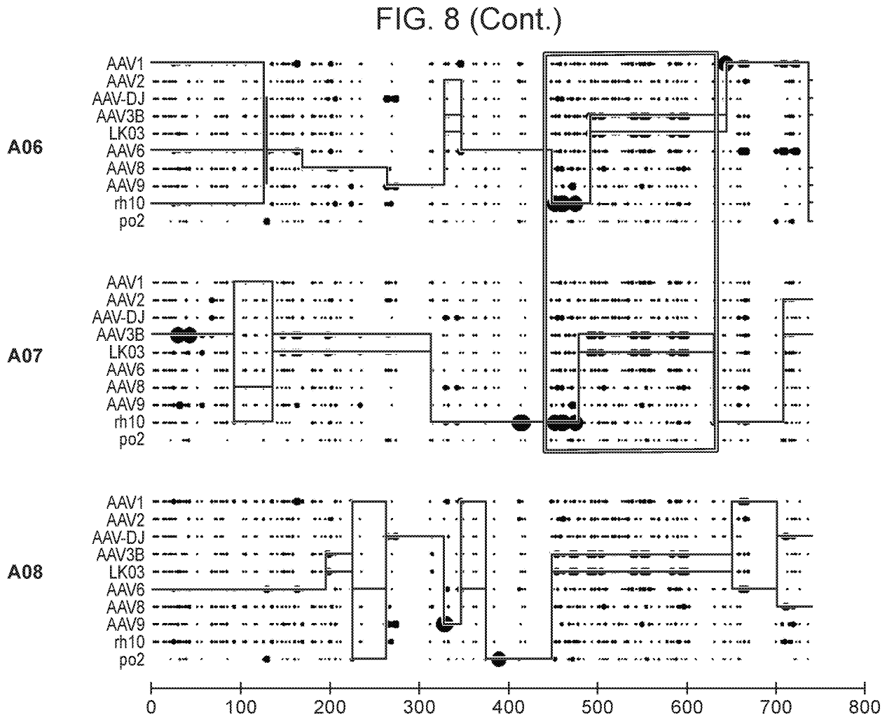

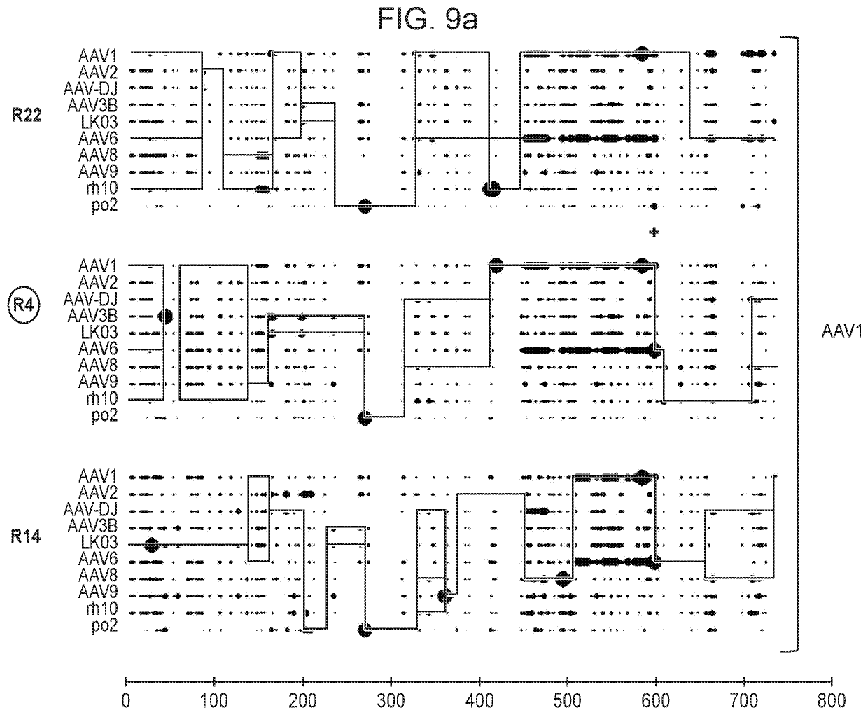

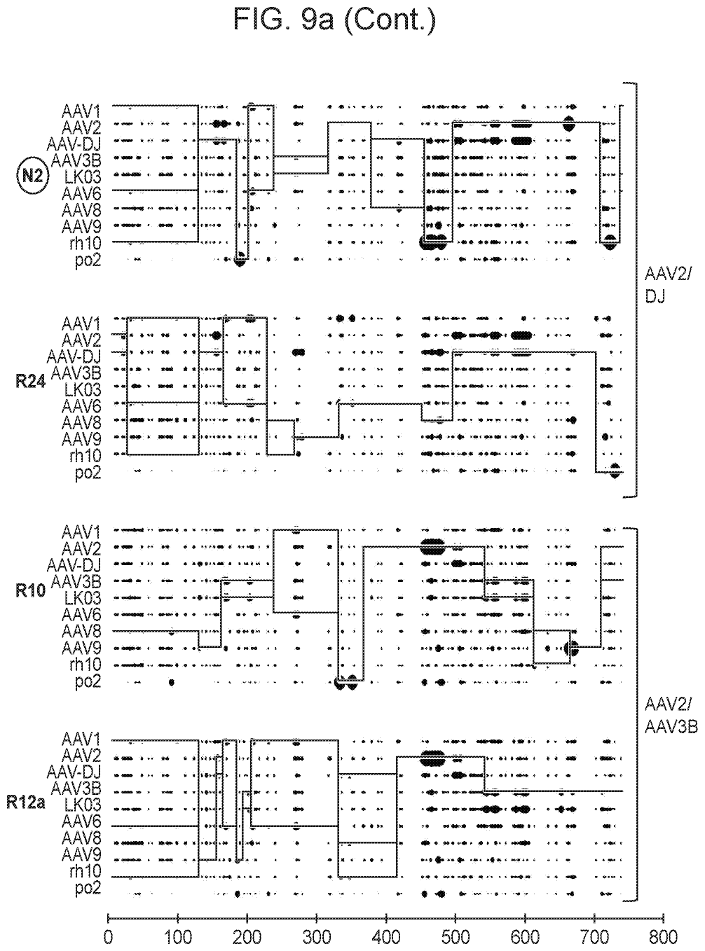

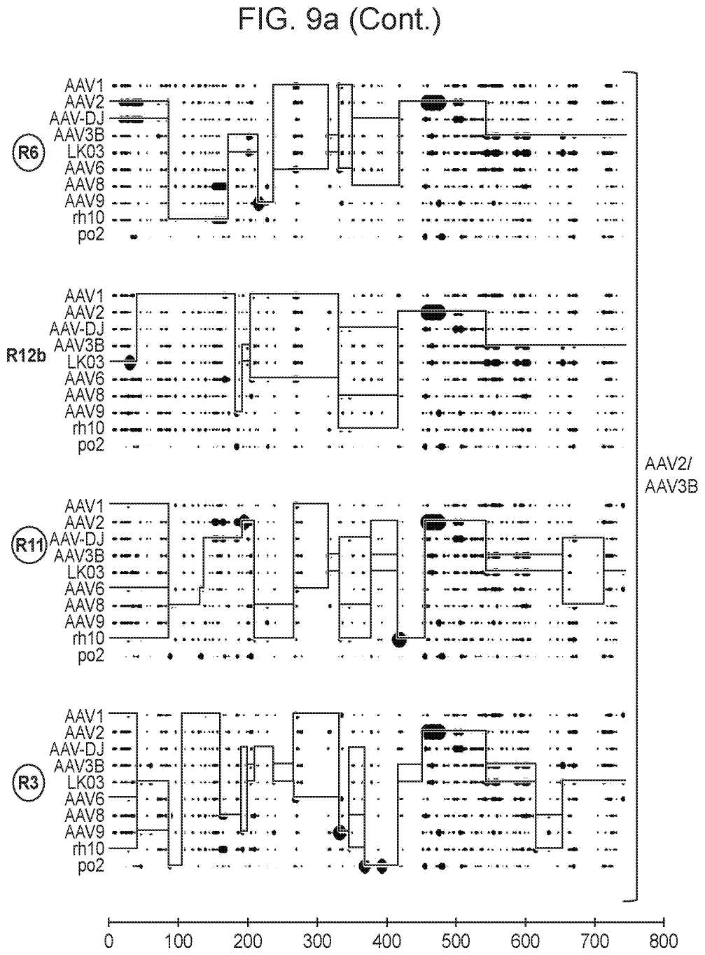

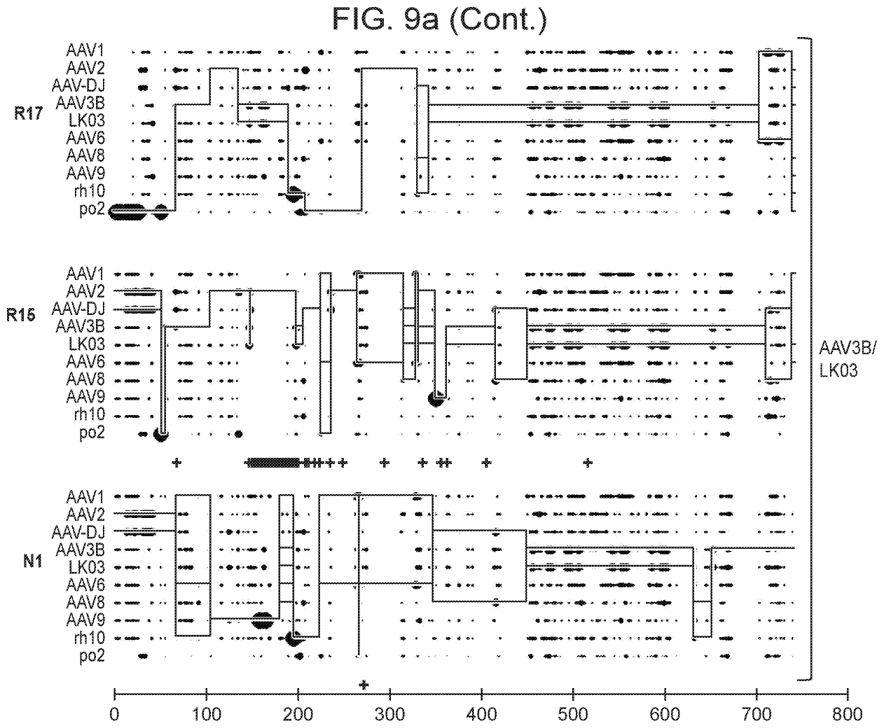

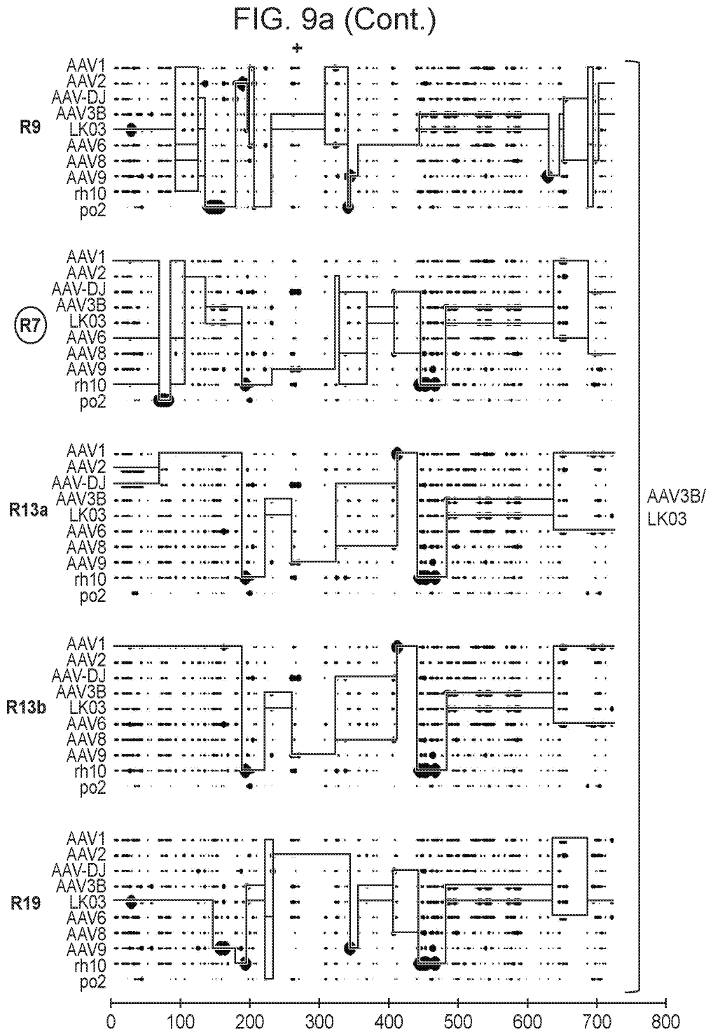

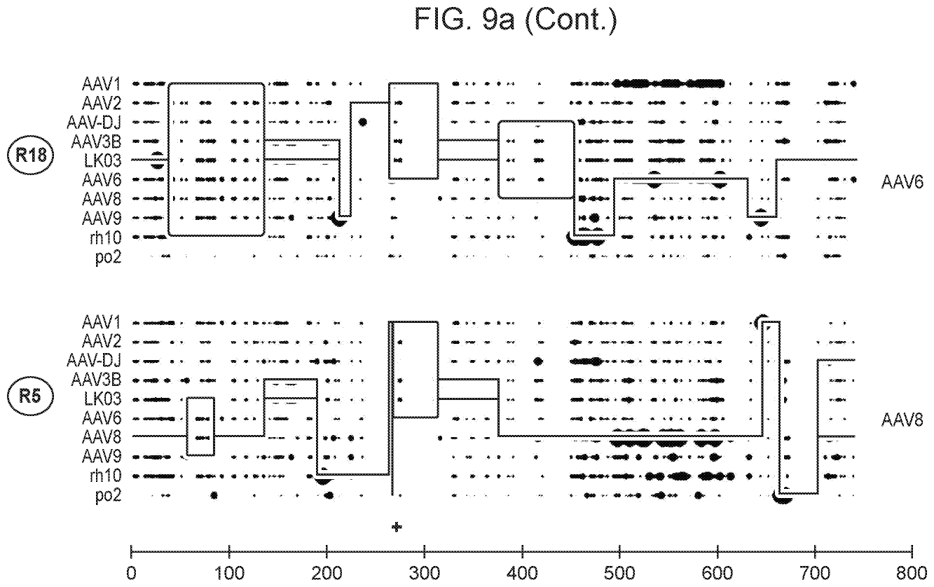

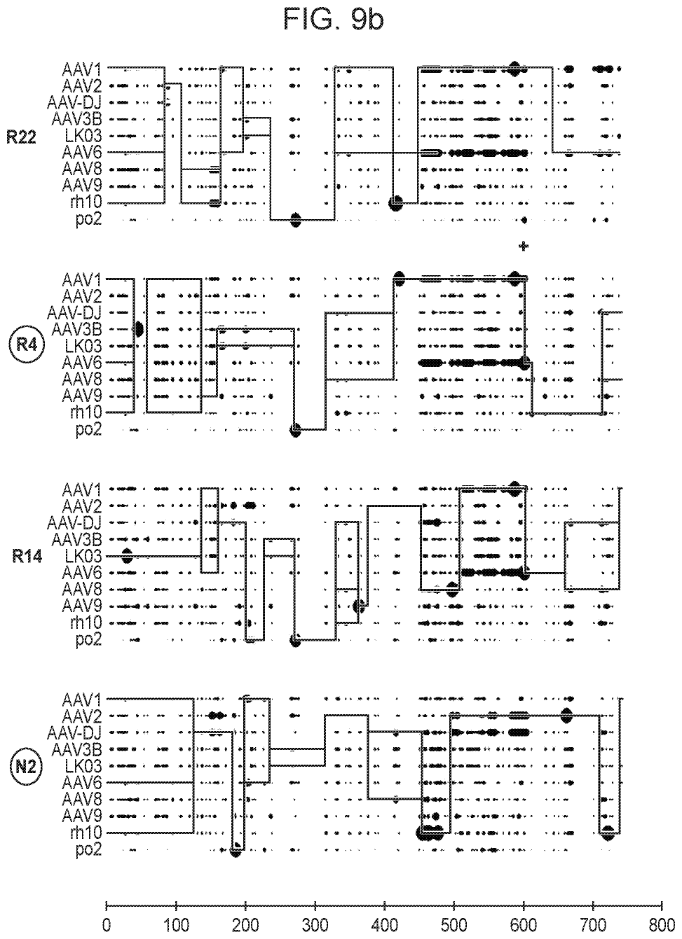

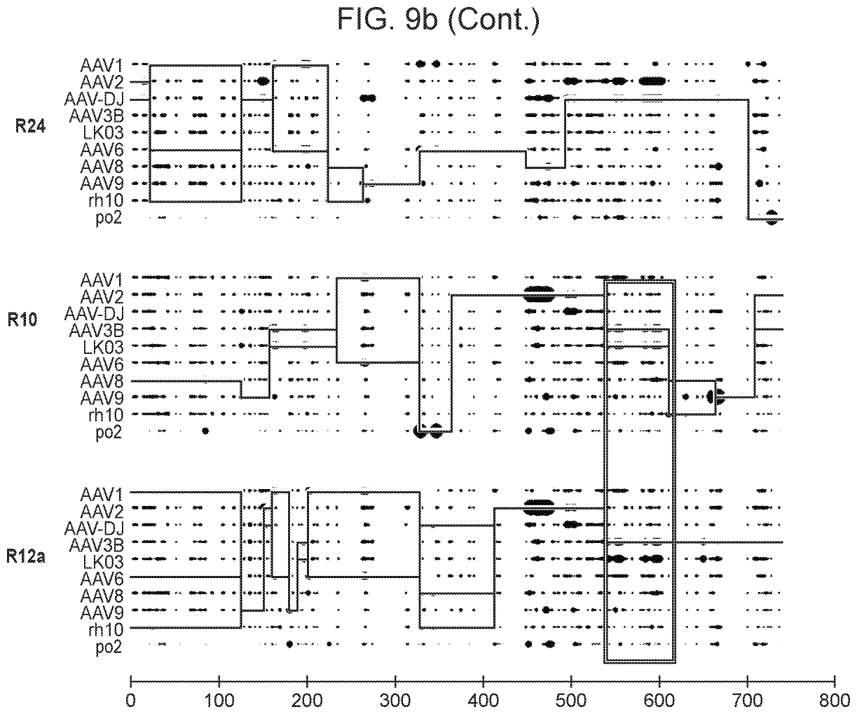

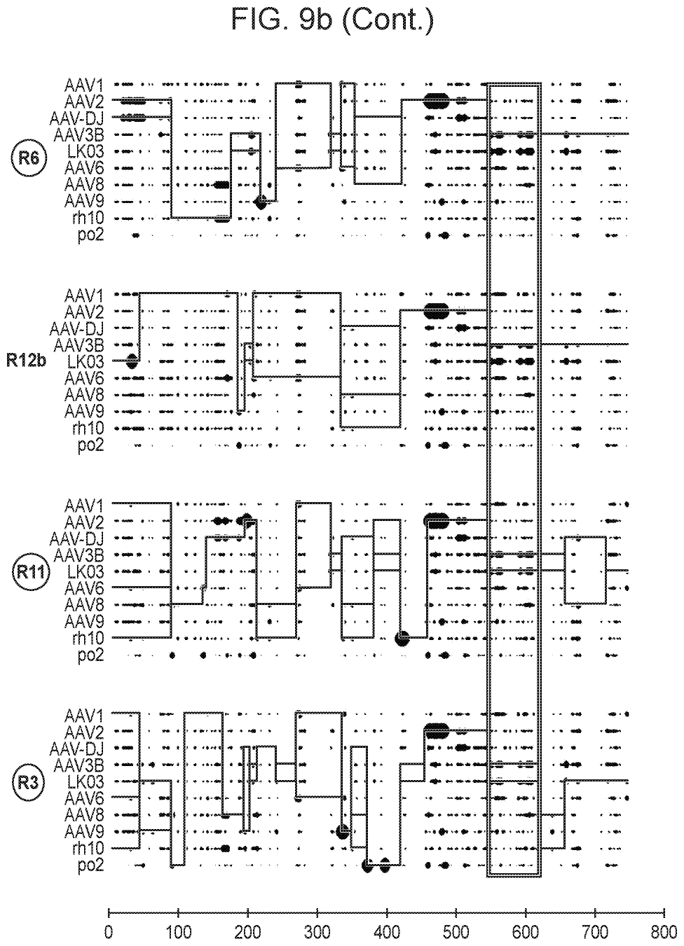

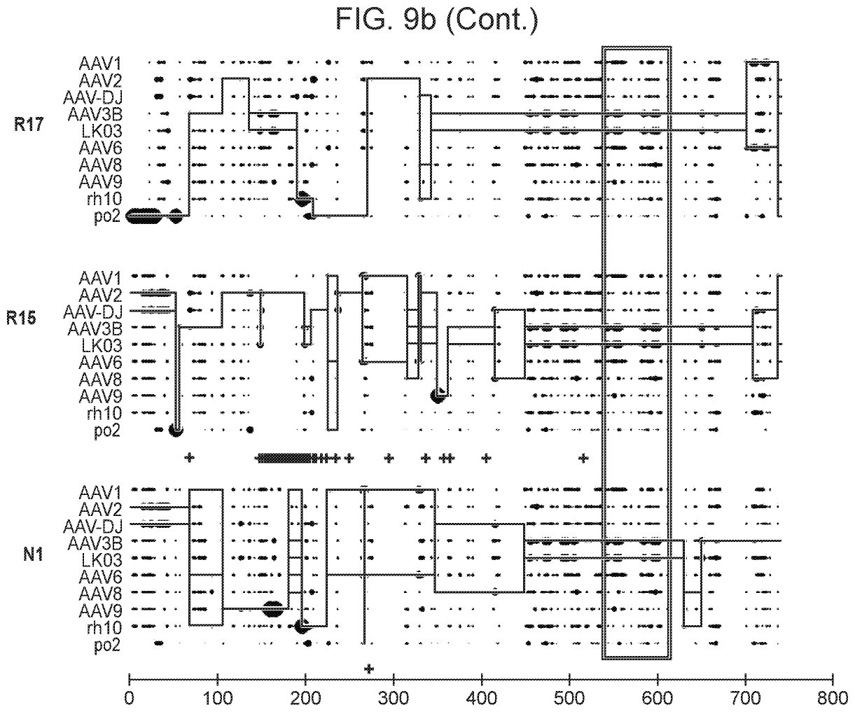

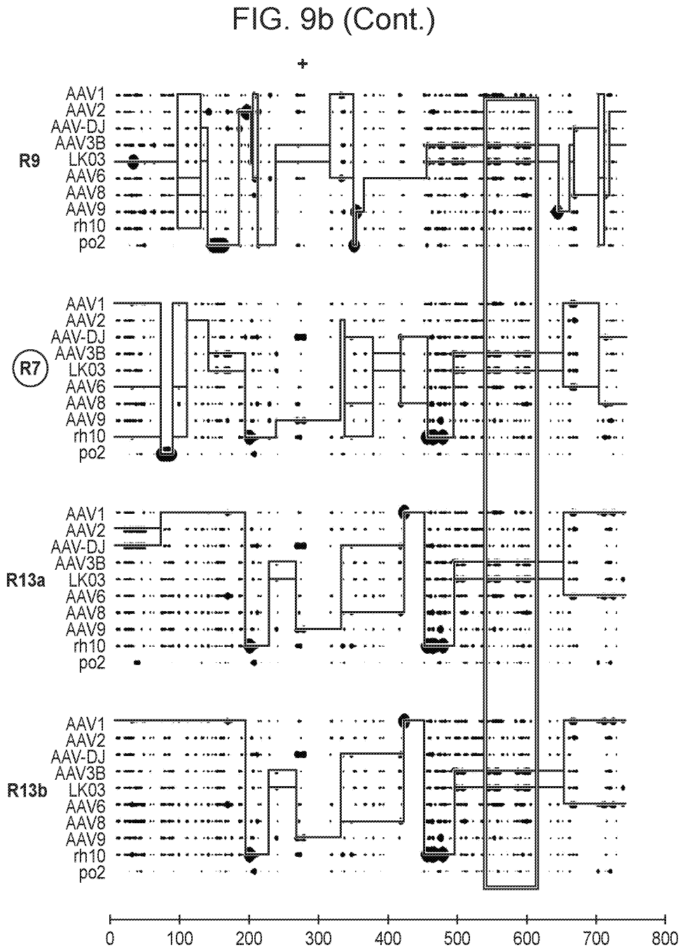

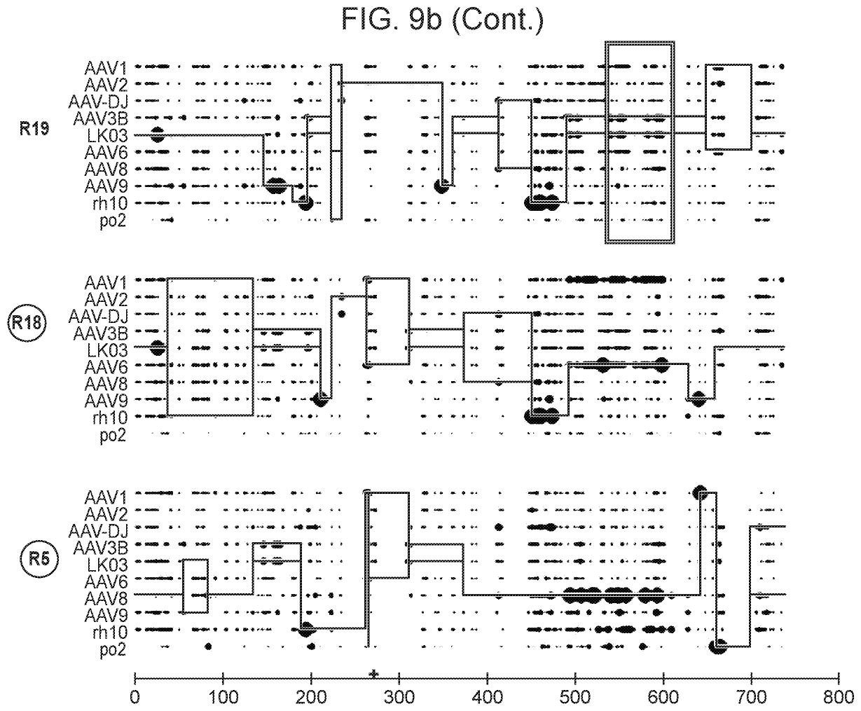

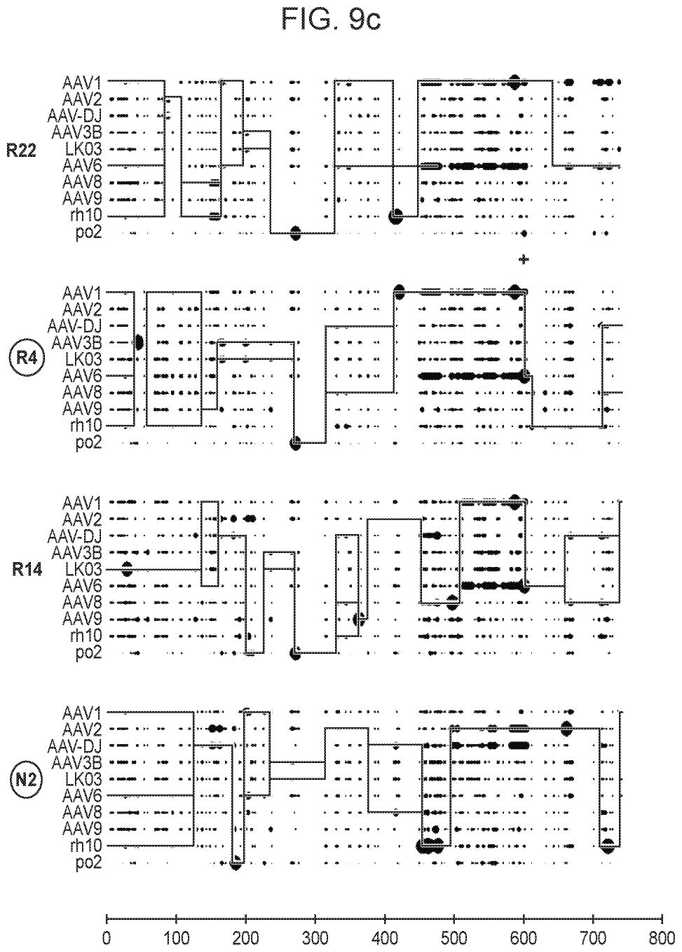

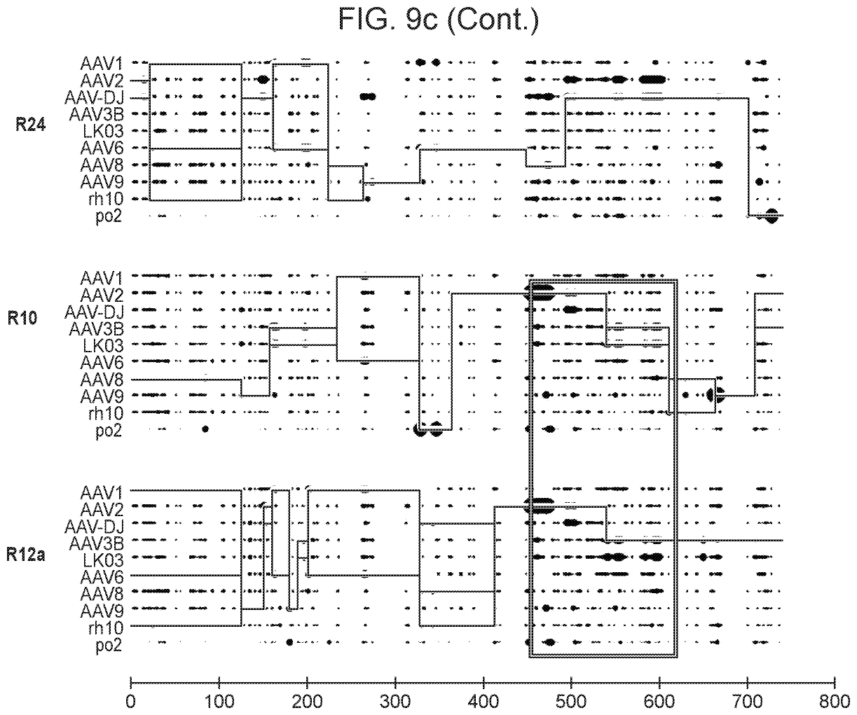

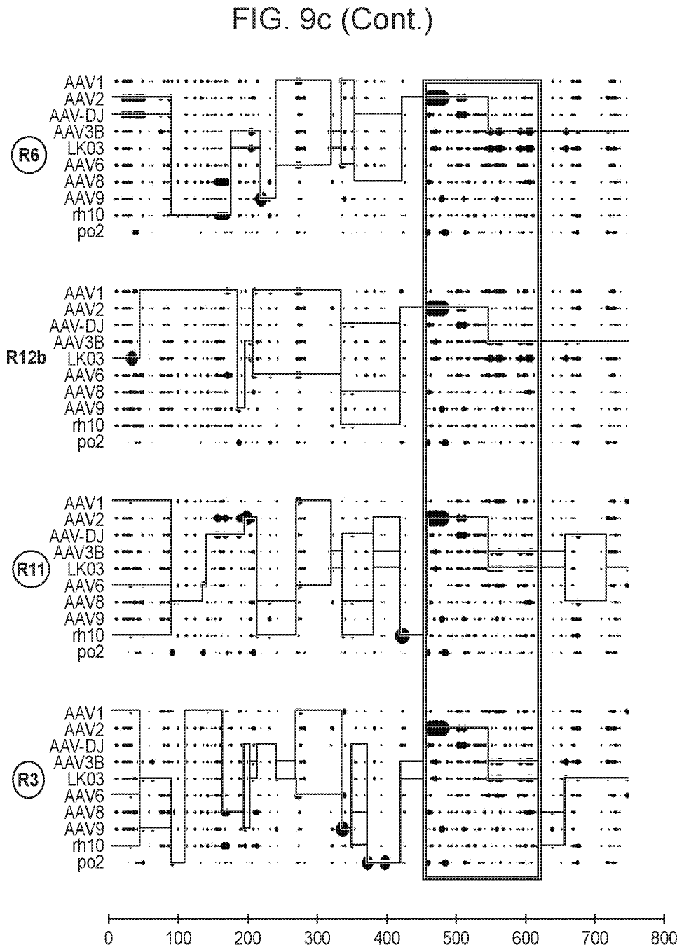

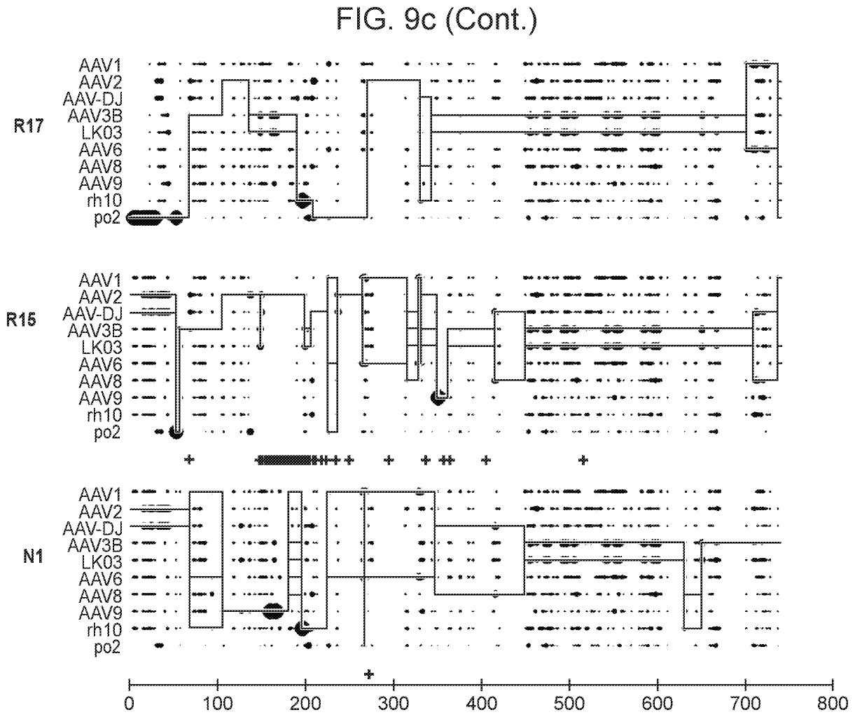

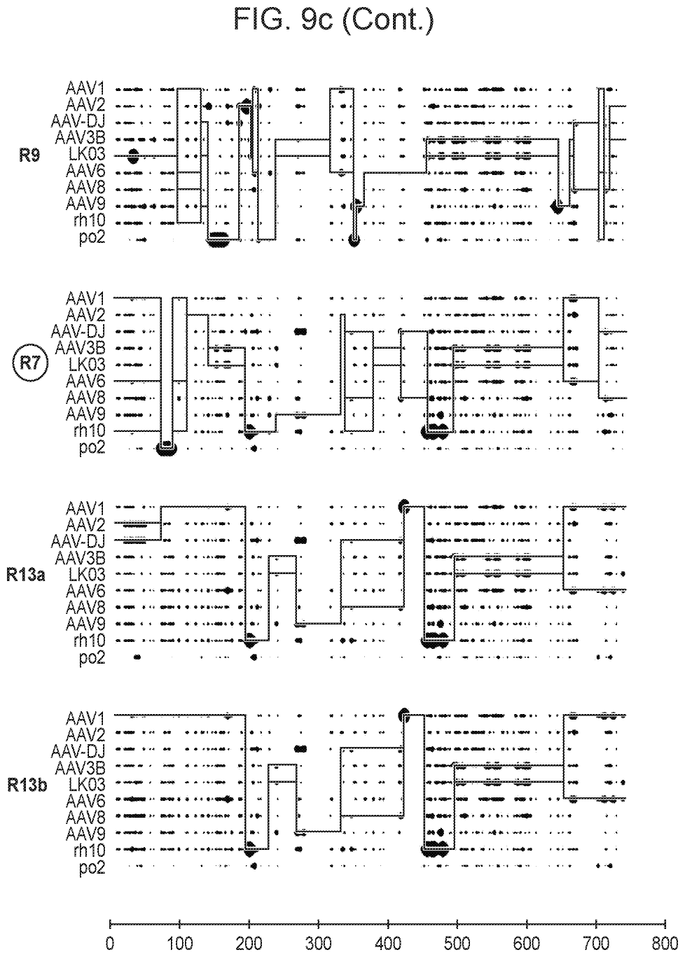

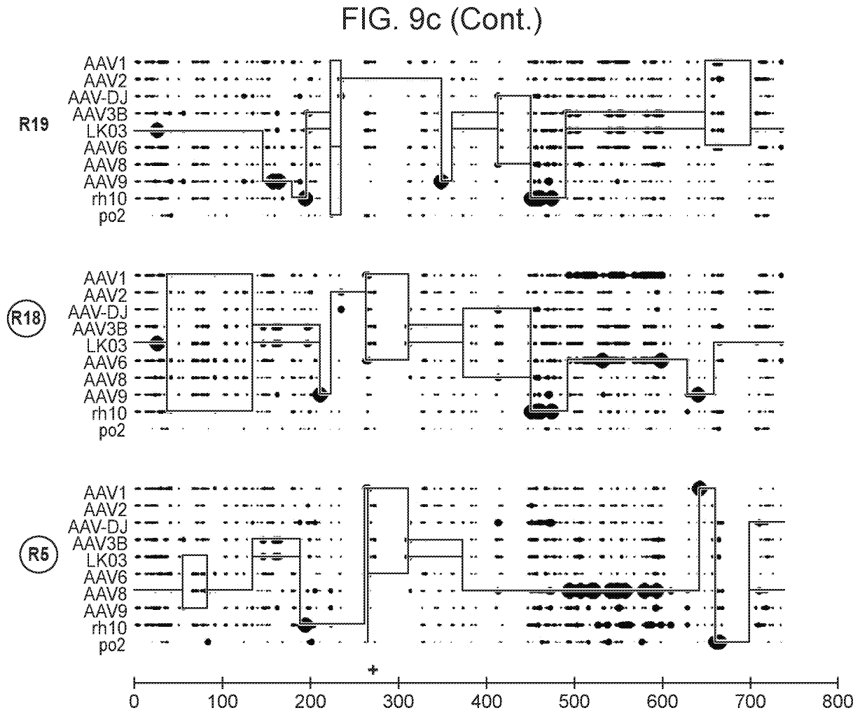

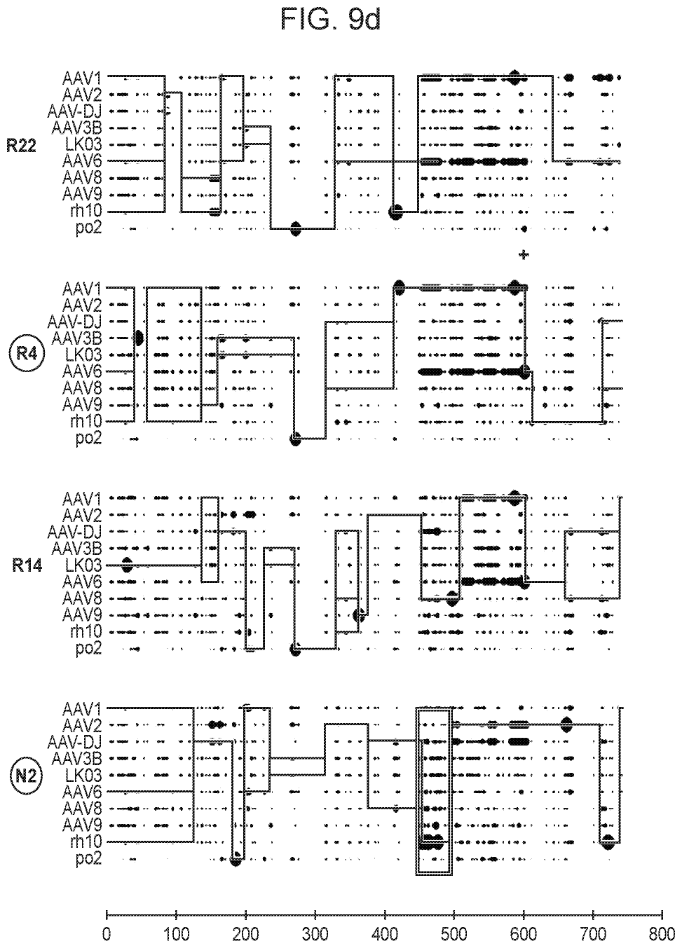

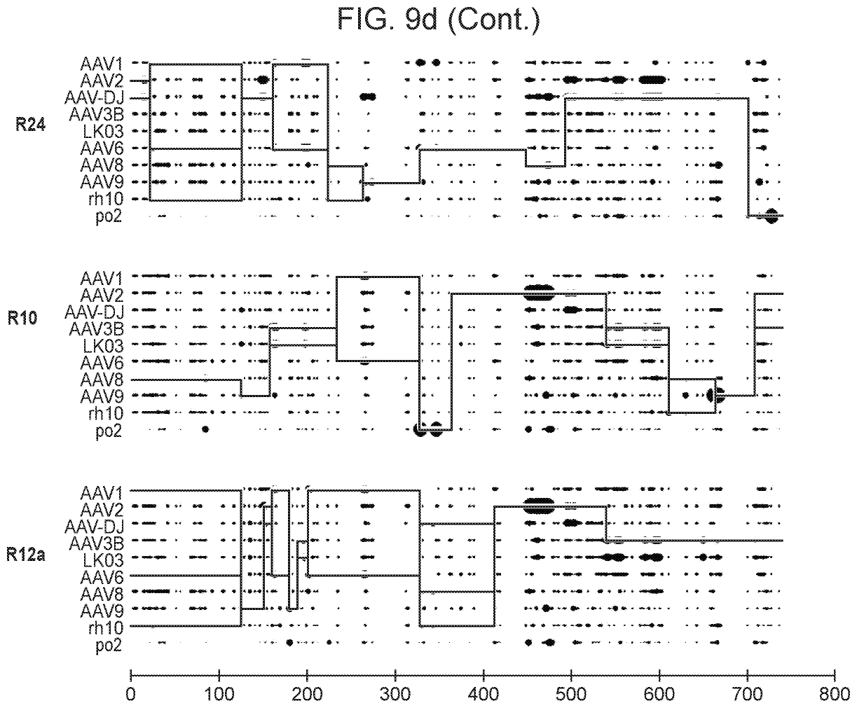

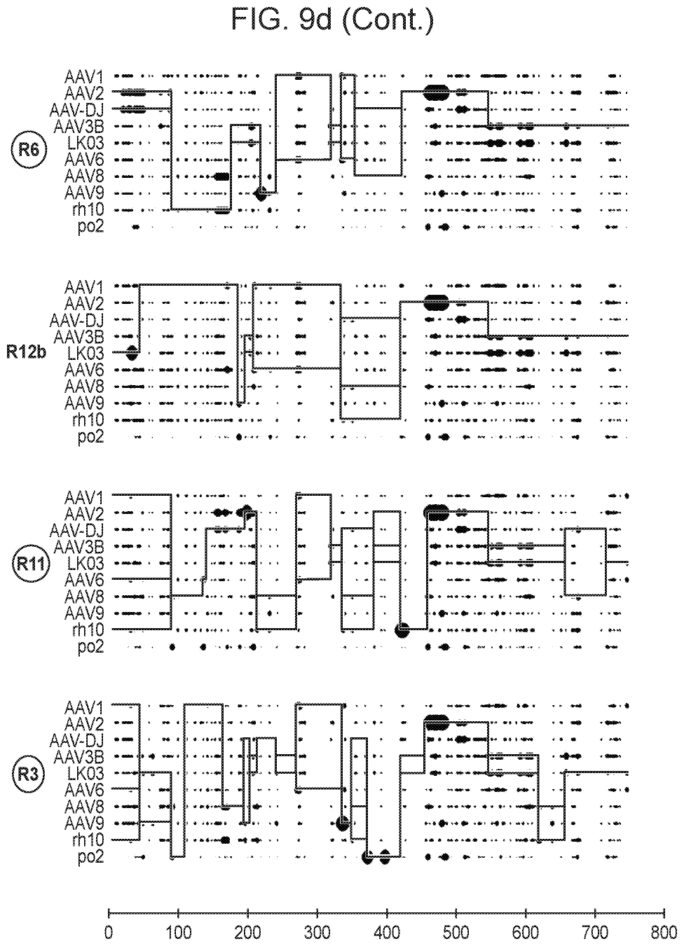

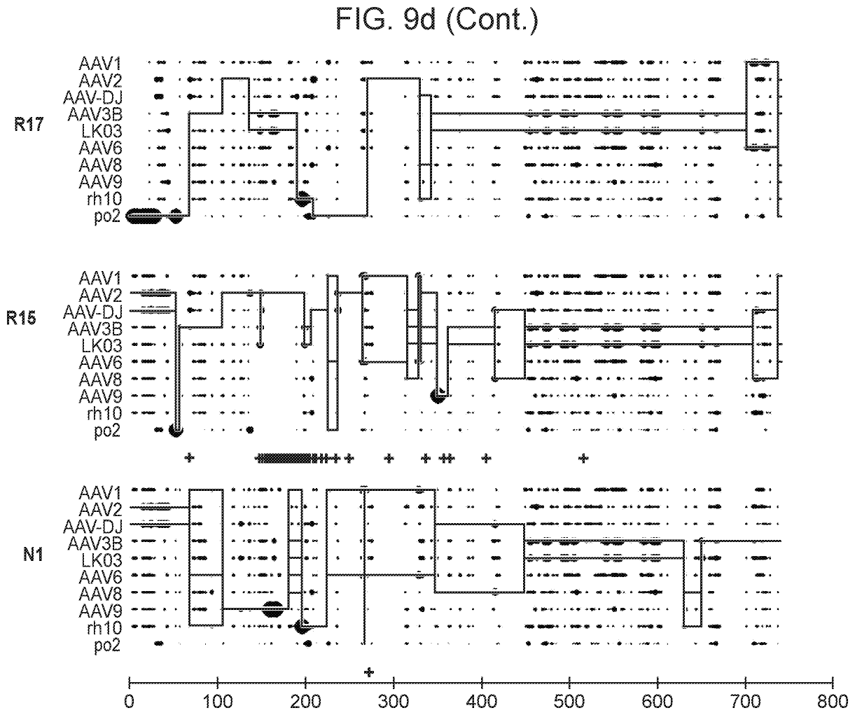

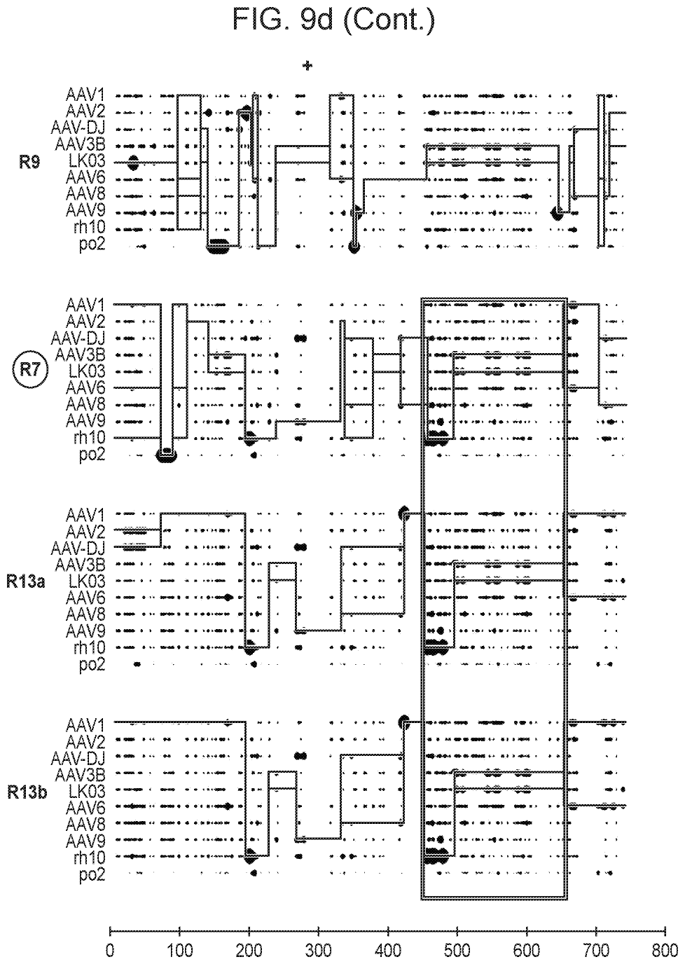

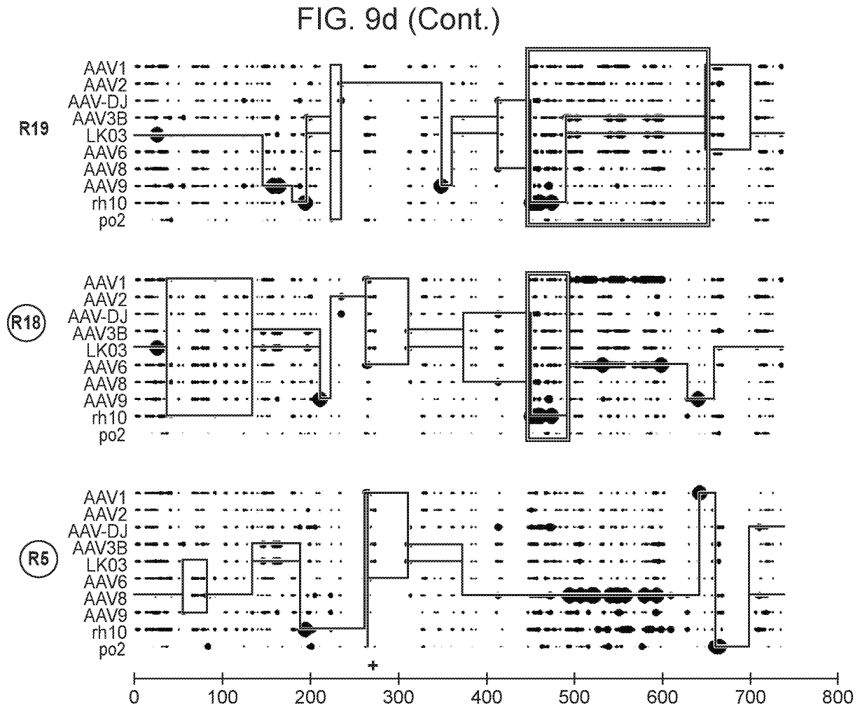

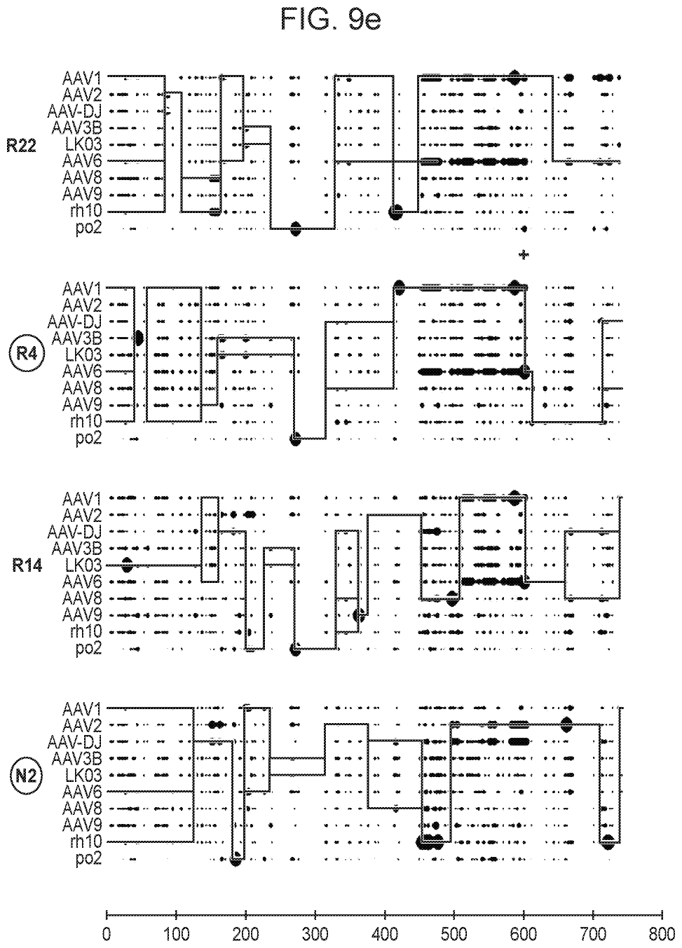

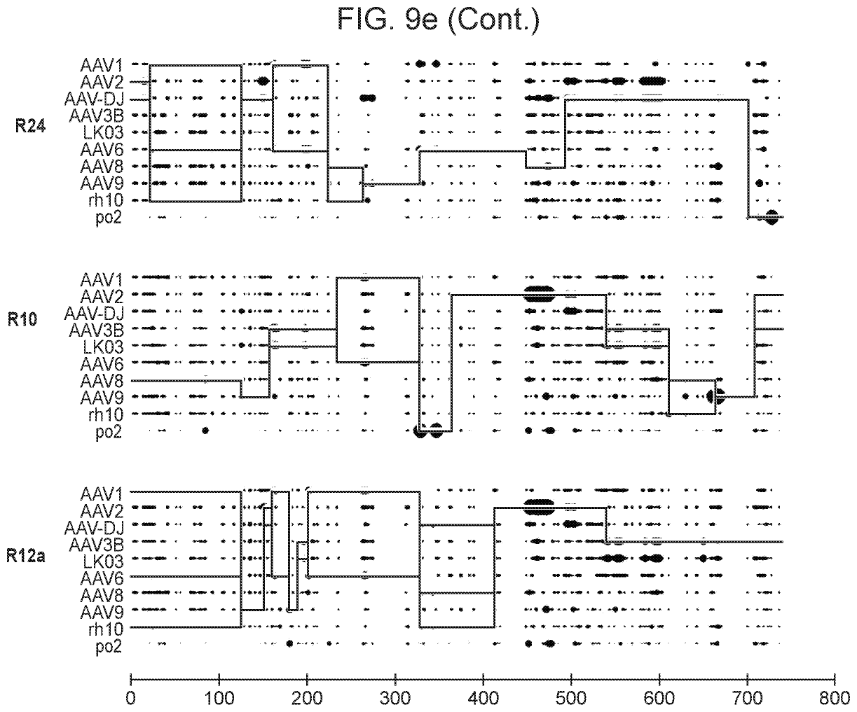

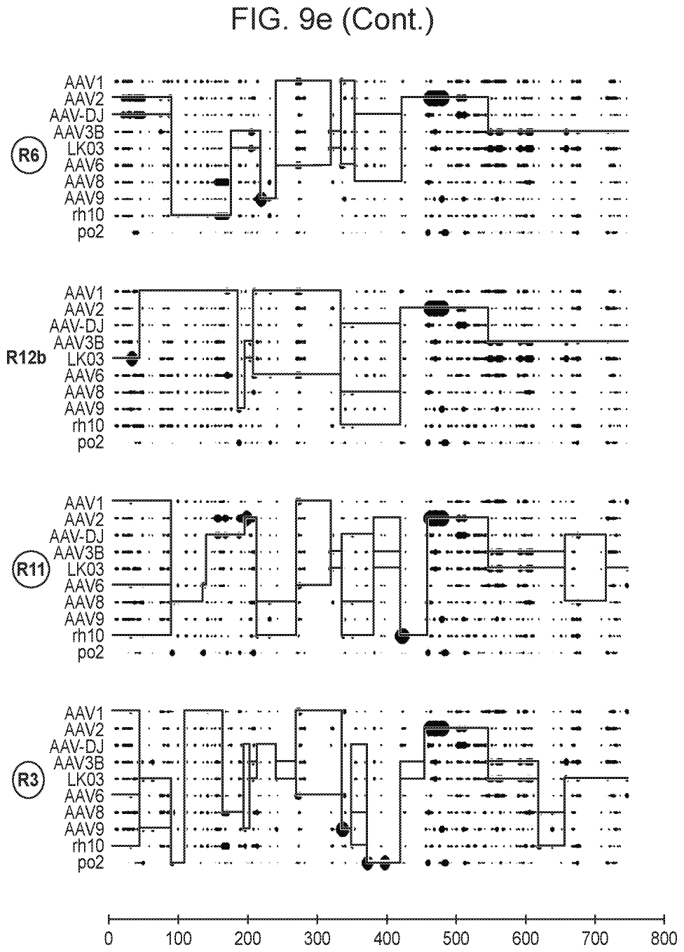

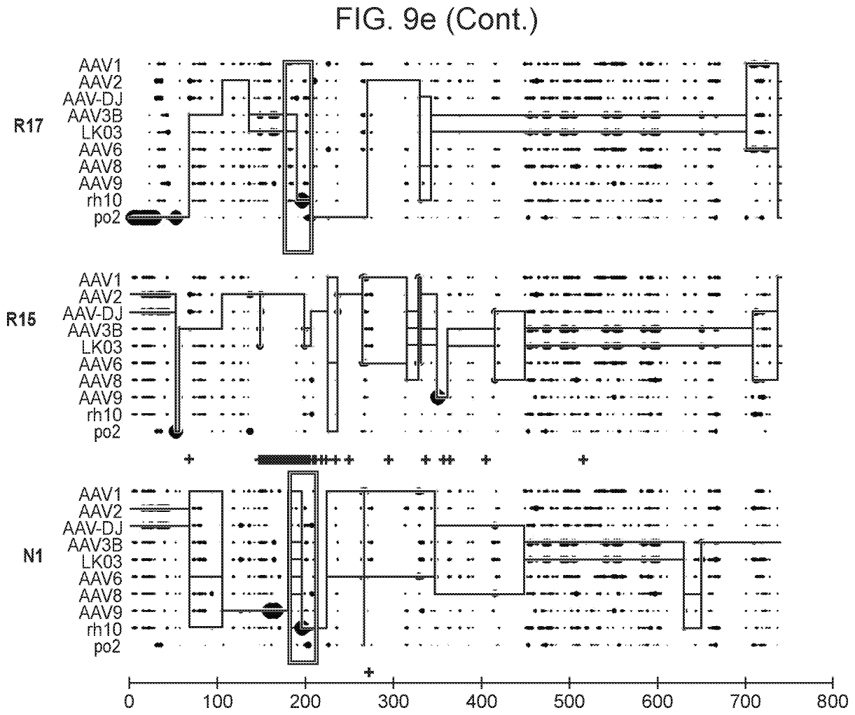

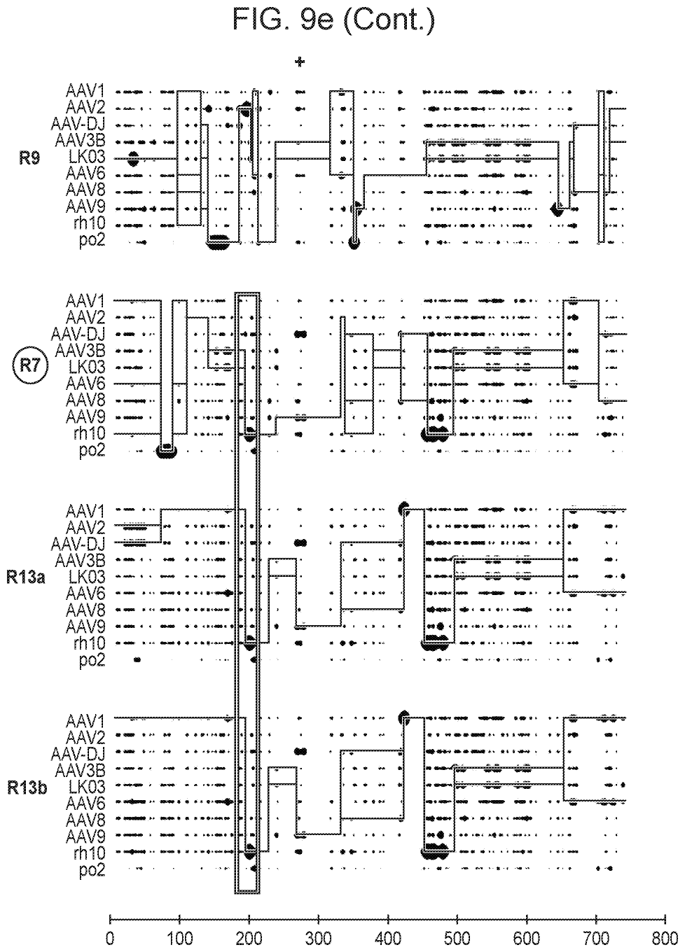

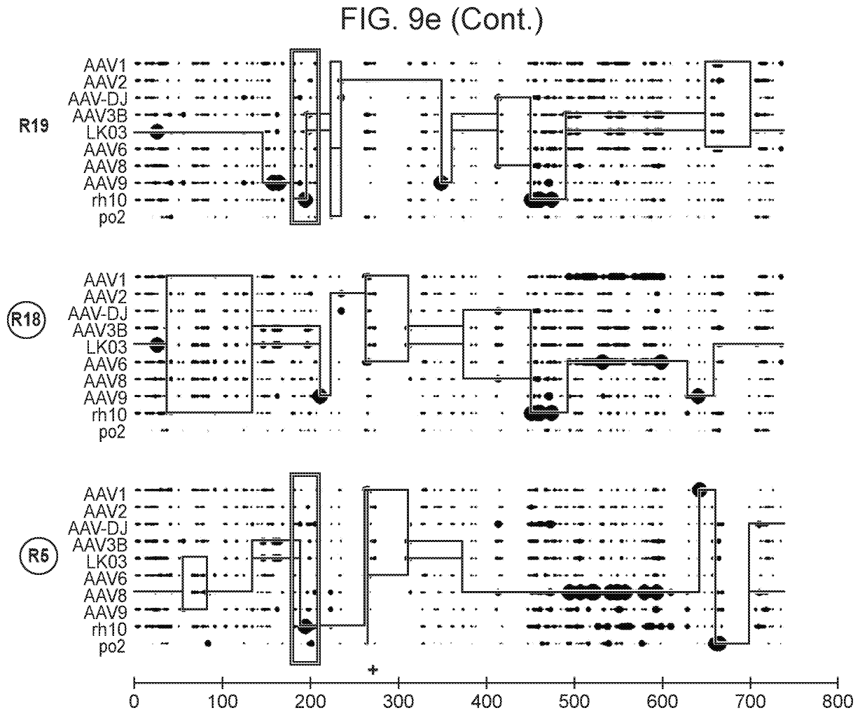

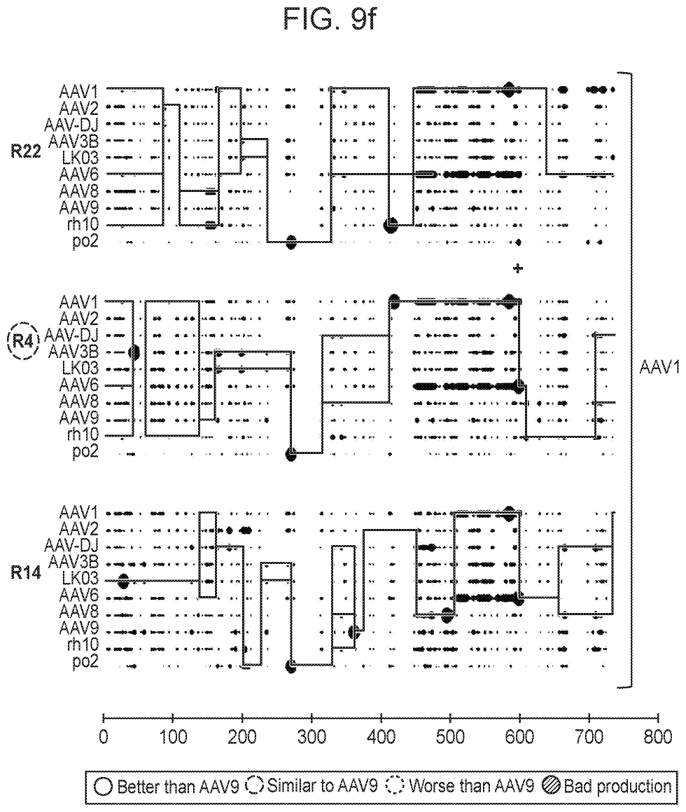

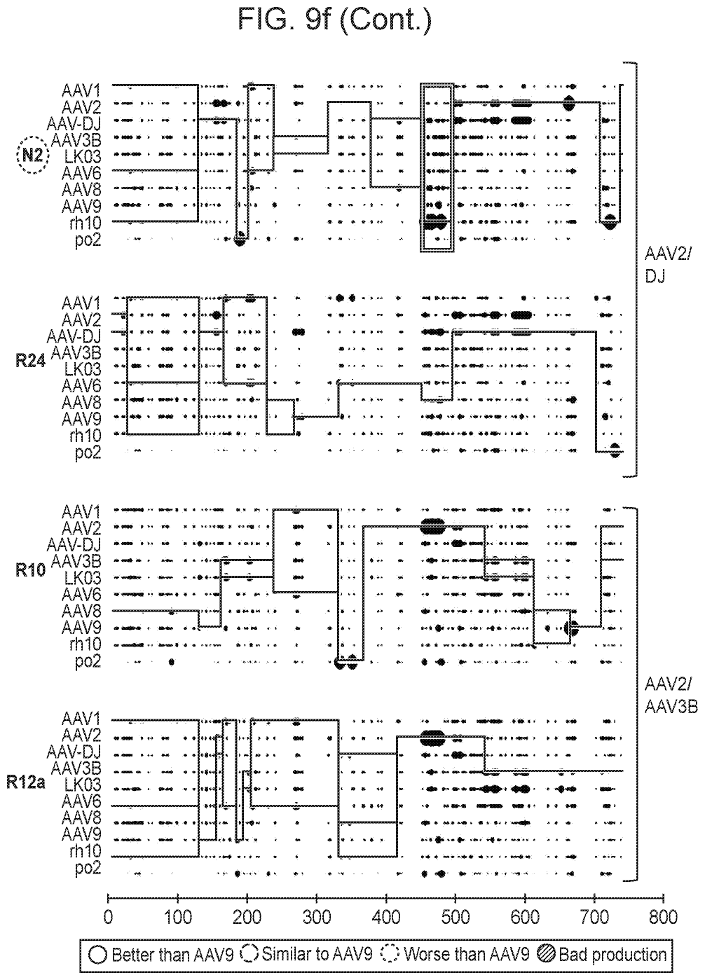

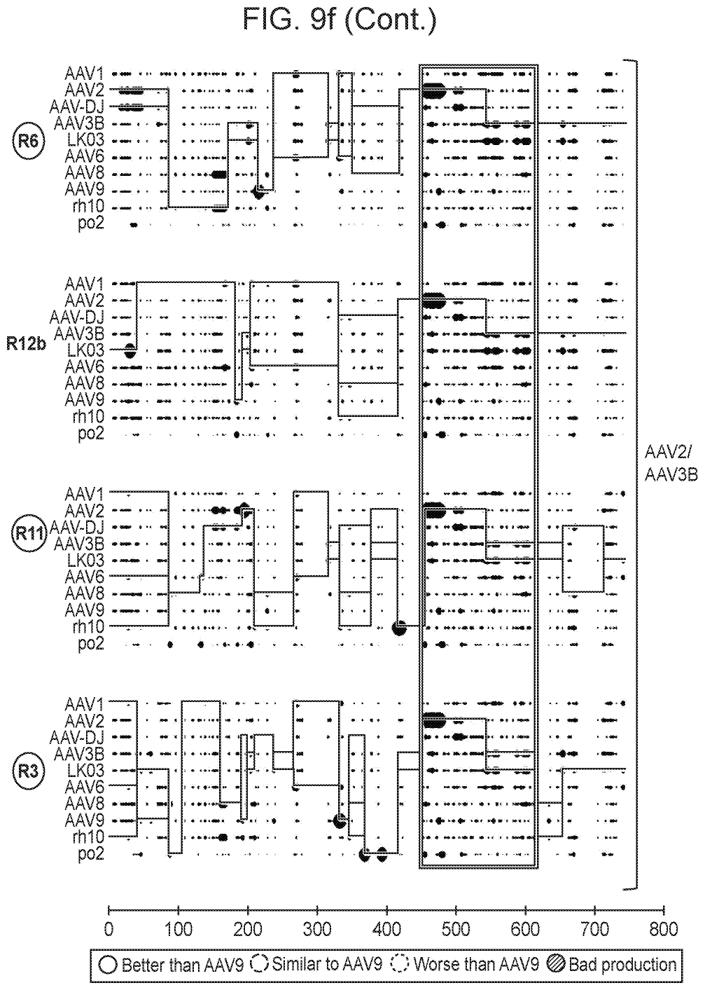

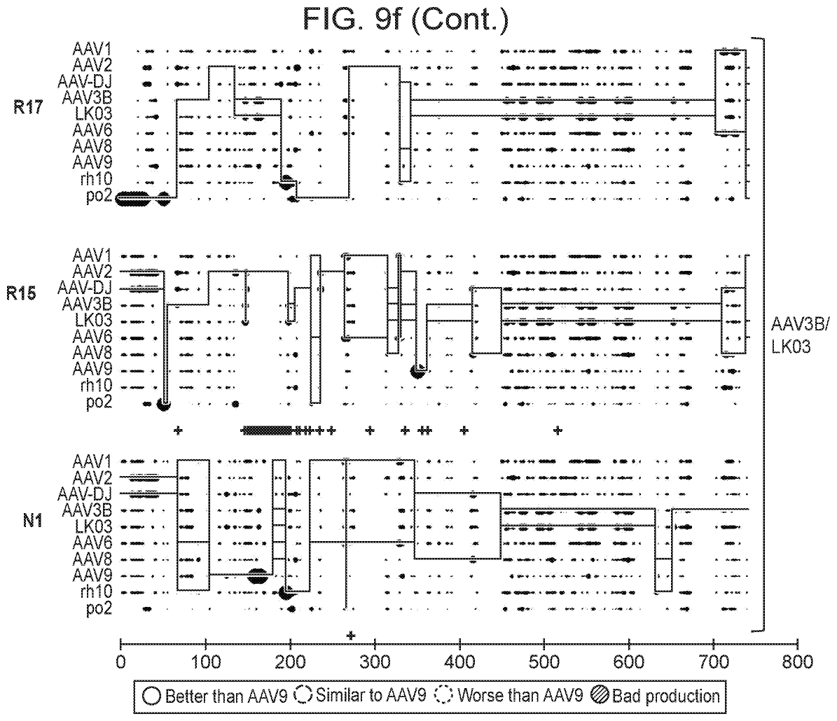

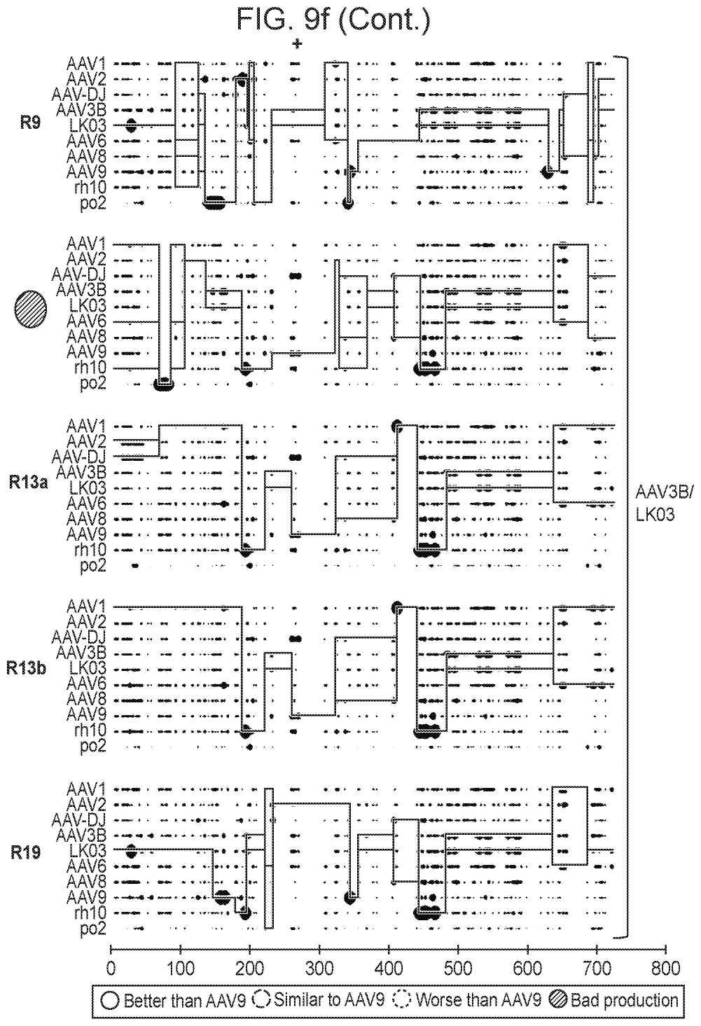

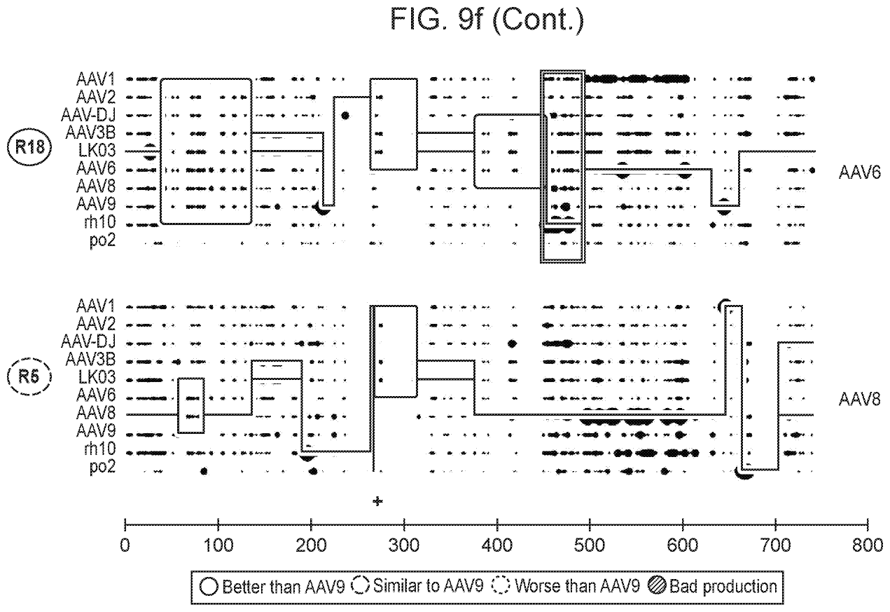

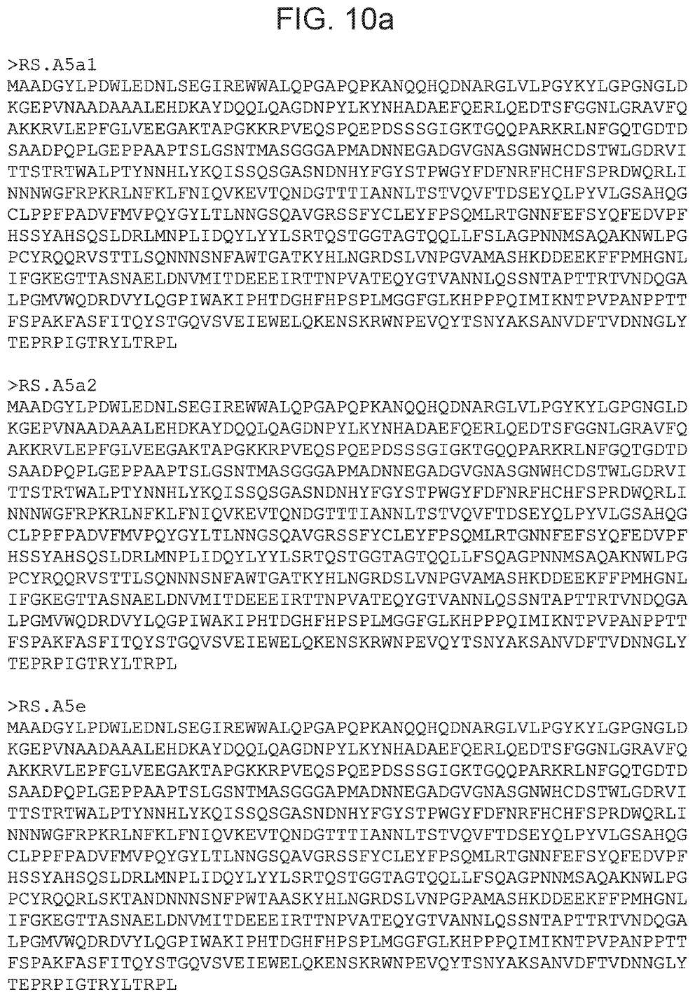

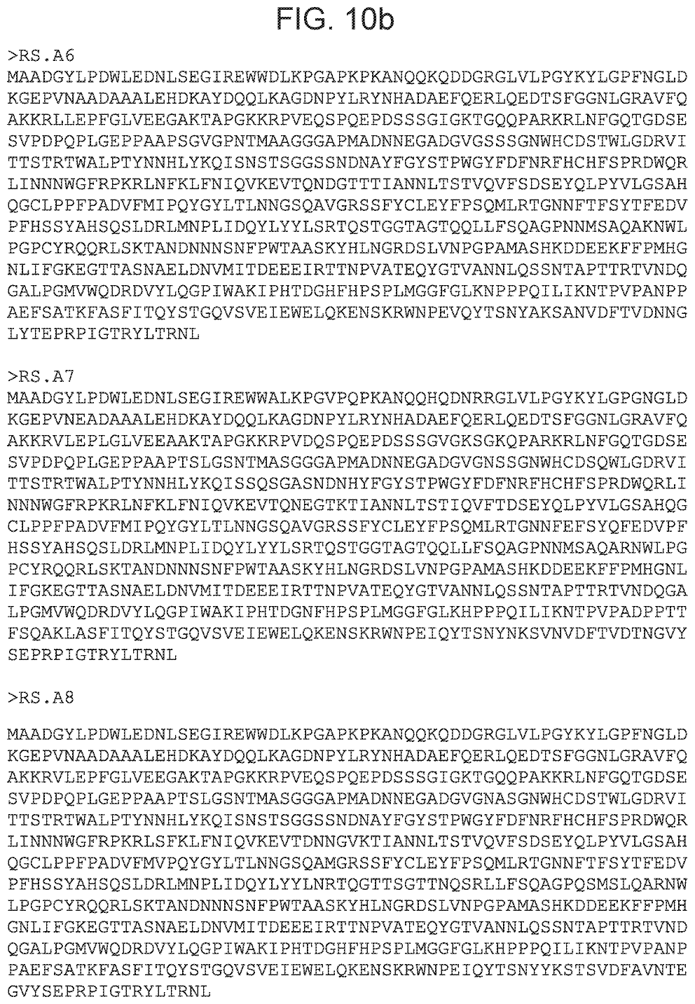

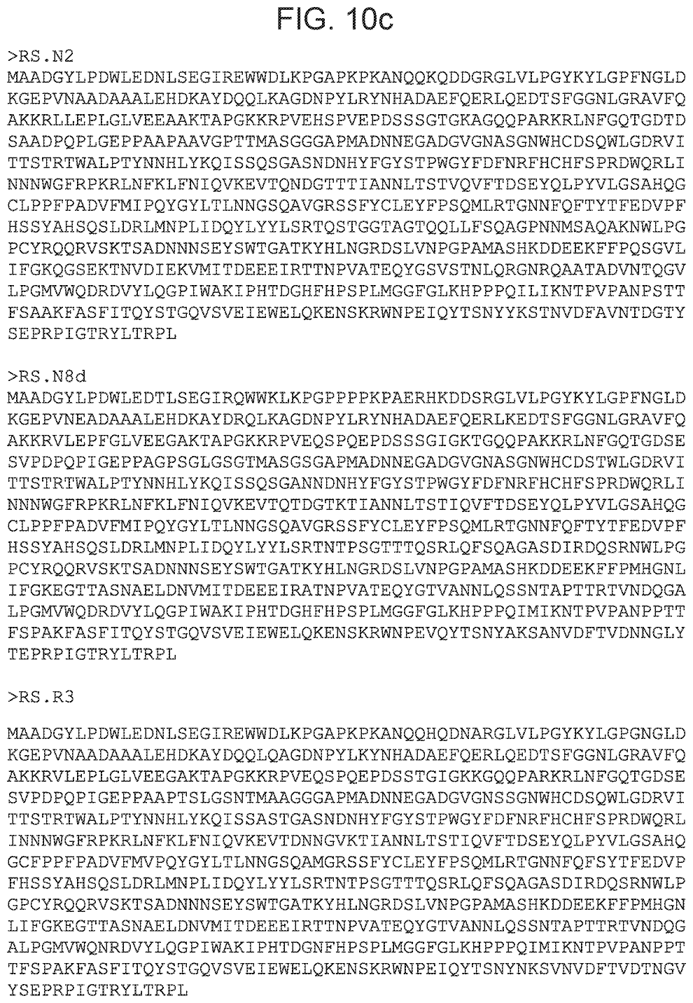

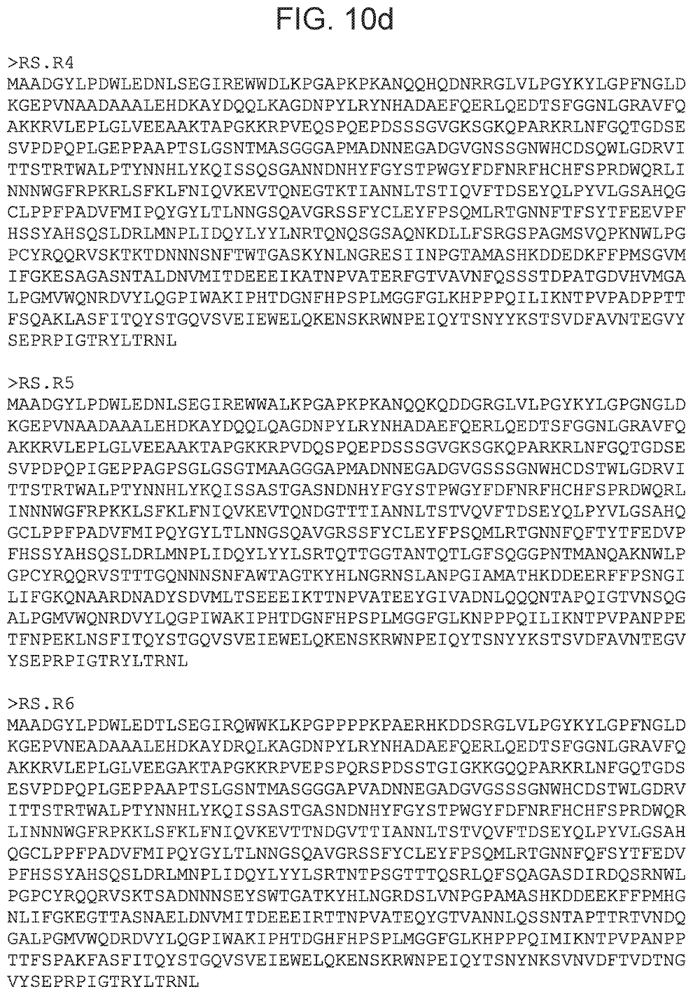

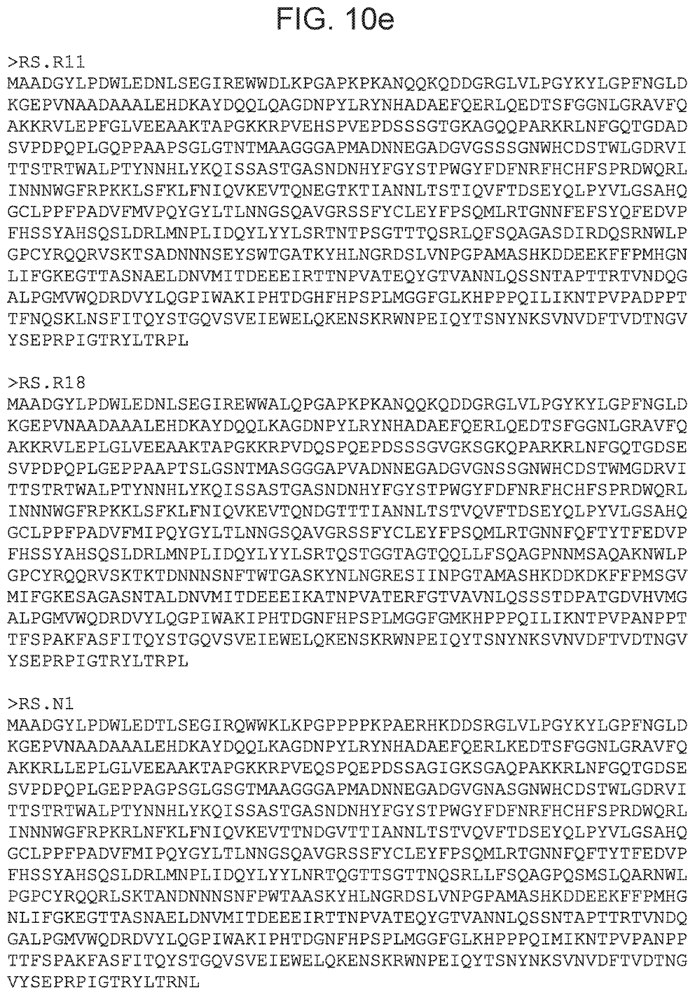

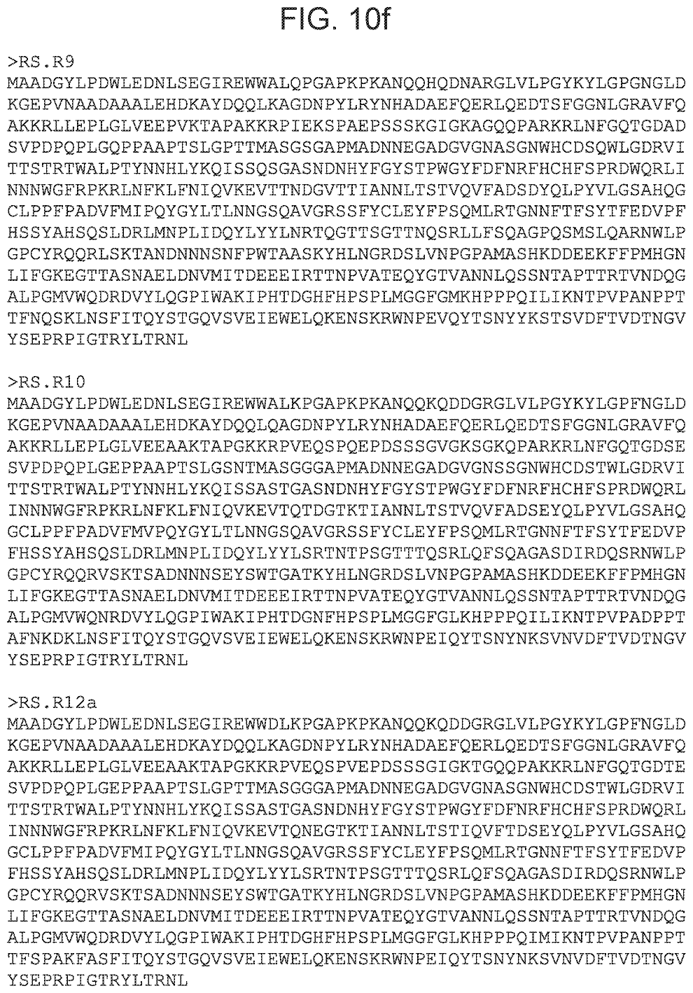

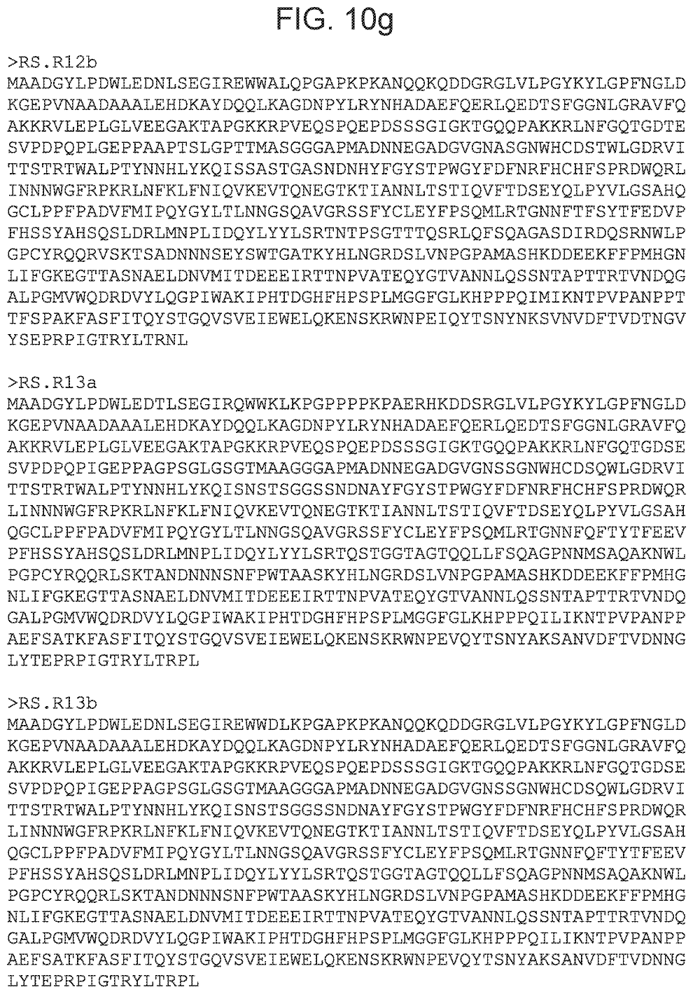

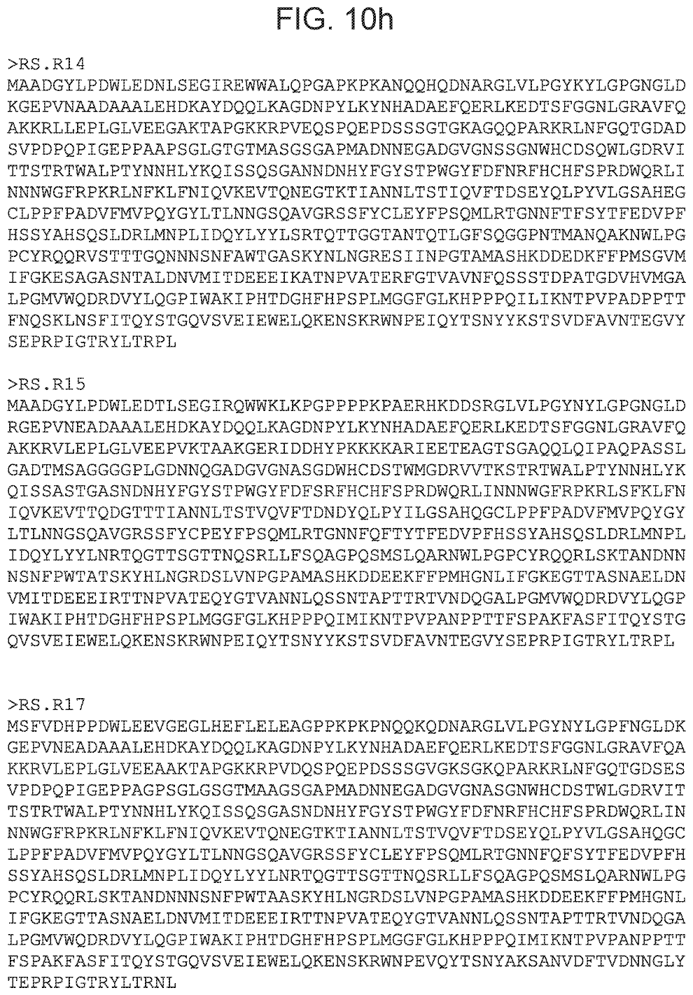

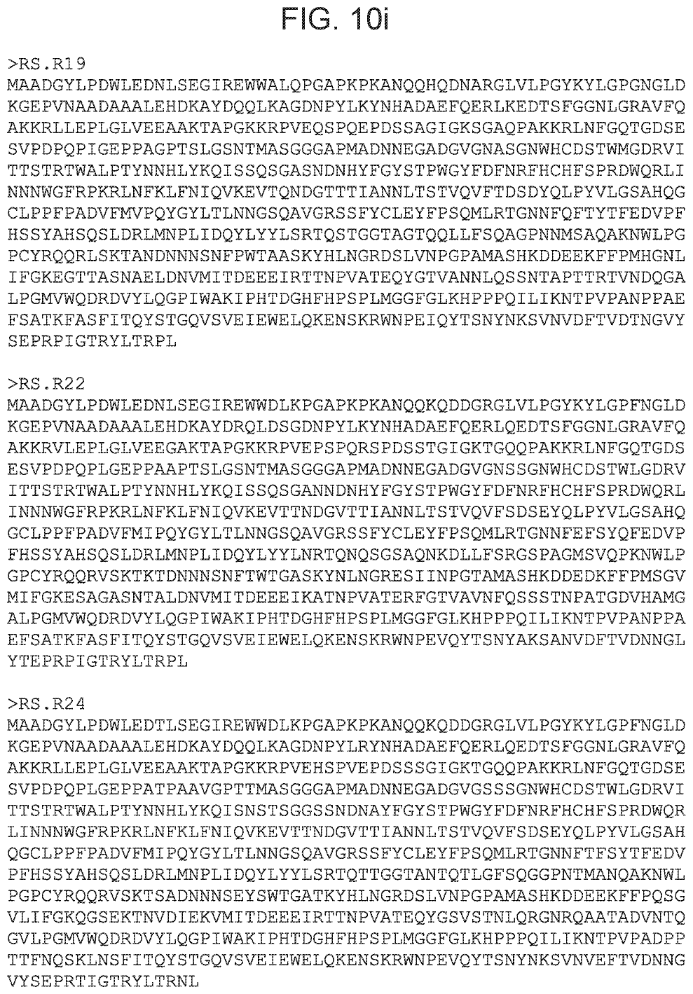

FIG. 8 and FIG. 9 depict sequence crossover (Xover) analyses of the identified AAV capsid proteins and FIG. 10 depicts the AAV capsid amino acid sequences. FIG. 8 depicts a crossover (Xover) analysis of 6 viruses selected from the Astrocytes with Ad5 selection. A5a1, A5a2 and A5e are originally the same virus in the selection, their differences are caused by PCR artifacts during sequencing sample preparation. All A5 viruses as well as A6 and A7 have similar parts in the red box, partially from AAV.rhesus10 parent and partially from AAV3B/LK03 parent, we hypothesize that this is what is required for the virus to be able to cross the endothelial cells, enter astrocytes, as well as cross in to the flowthough. A8, on the other hand, does not have the AAV.rhesus10 contribution right before AAV3B/LK03, and thus performs very similar to AAV3B/LK03 in the transwell in entering the astrocytes, but does not cross into the flowthrough.

FIG. 9a-9f depict a crossover (Xover) analysis of sequences selected in neurons. (FIG. 9a) This and the follow figures show the Xover pattern analysis of the 19 sequences that were found that highly increased in the selection in neurons with replication in a preliminary PacBio sequence analysis. The total sequence actually is 21, but 2 of the sequences have 2 isoforms (labeled as R12a and R12b, and R13a and R13b in the figure) due to PCR artifacts. The sequences are arranged as labeled in FIG. 9a and FIG. 9f) by which parent contributed the most in the C-terminal of the virus sequences. Certain parental contributions appeared with different probability in different parts of the viruses [FIG. 9b: 12/19 had AAV3B at ˜aa530-610; FIG. 9c: 5/19 had AAV2+AAV3B at ˜aa450-610; FIG. 9d: 5/19 had AAVrh10 at ˜aa450-500; FIG. 9e: 6/19 had AAVrh10 at ˜aa200]. FIG. 9f depicts a summary of the previous figures (all 19 viruses selected from Neurons with Ad5 selection). The circles/arrows refer to how they performed in the transwell assay, and boxes show which parent contribution may have been responsible for their phenotype. 7 of the sequences with varying patterns were picked for the analyses described in the previous figures. Conclusions: (1) 4 crossed BBB more efficient than AAV9, 2 crossed BBB similar to AAV9, 1 crossed BBB less efficient AAV9, and 1 did not produce high virus titer; (2) AAV regions that may facilitate crossing BBB: AAV2 (˜aa450-550)+AAV3B (˜aa550-610).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A variant adeno-associated virus (AAV) capsid protein that provides a viral particle with the ability to traverse the human blood brain barrier (BBB) and transduce cells of the central nervous system (CNS), wherein the variant AAV capsid protein comprises an amino acid sequence having 97% or more sequence identity with the amino acid sequence set forth in any one of SEQ ID NOs: 1-27.

2. The variant AAV capsid protein of claim 1, wherein the variant AAV capsid protein comprises an amino acid sequence having 97% or more sequence identity with the amino acid sequence set forth in any one of SEQ ID NOs: 1-14.

3. The variant AAV capsid protein of claim 1, wherein the variant AAV capsid protein comprises an amino acid sequence having 97% or more sequence identity with the amino acid sequence set forth in any one of SEQ ID NOs: 1-6.

4. The variant AAV capsid protein of claim 1, wherein the variant AAV capsid protein comprises an amino acid sequence having 97% or more sequence identity with the amino acid sequence set forth in any one of SEQ ID NOs: 7-14.

5-7. (canceled)

8. The variant AAV capsid protein of claim 1, wherein the variant AAV capsid protein comprises an amino acid sequence having 97% or more sequence identity with the amino acid sequence set forth in any one of SEQ ID NOs: 3, 4, and 11.

9. The variant AAV capsid protein of claim 1, wherein the variant AAV capsid protein comprises an amino acid sequence having 97% or more sequence identity with the amino acid sequence set forth in any one of SEQ ID NOs: 15-27.

10. A variant adeno-associated virus (AAV) capsid protein that provides a viral particle with the ability to traverse the human blood brain barrier (BBB) and transduce cells of the CNS, wherein the variant AAV capsid protein comprises an amino acid sequence having 95% or more sequence identity with the amino acid sequence set forth in any one of SEQ ID NOs: 1-4, 6-10, 12-24, and 27.

11. The variant AAV capsid protein of claim 10, wherein the variant AAV capsid protein comprises an amino acid sequence having 95% or more sequence identity with the amino acid sequence set forth in any one of SEQ ID NOs: 8-9 and 12-14.

12. The variant AAV capsid protein of claim 10, wherein the variant AAV capsid protein comprises an amino acid sequence having 95% or more sequence identity with the amino acid sequence set forth in any one of SEQ ID NOs: 9 and 12-14.

13. The variant AAV capsid protein of claim 10, wherein the variant AAV capsid protein comprises an amino acid sequence having 95% or more sequence identity with the amino acid sequence set forth in any one of SEQ ID NOs: 15-24, and 27.

14. A nucleic acid comprising a nucleotide sequence that encodes the variant AAV capsid protein of claim 1.

15. The nucleic acid of claim 14, further comprising a nucleotide sequence of interest flanked by inverted terminal repeat sequences (ITRs).

16. The nucleic acid of claim 15, wherein the nucleotide sequence of interest encodes a polypeptide or a non-coding RNA.

17-18. (canceled)

19. A cell comprising the nucleic acid of claim 14.

20. A recombinant AAV particle comprising:
(a) the variant AAV capsid protein of claim 1; and
(b) a nucleic acid payload of interest.

21. The recombinant AAV particle of claim 20, wherein the nucleic acid payload of interest is a non-coding RNA, encodes said non-coding RNA, or encodes a polypeptide.

22. The recombinant AAV particle of claim 21, wherein the polypeptide is a genome-editing enzyme or a therapeutic protein.

23-27. (canceled)

28. A cell comprising the recombinant AAV particle of claim 1.

29. A method of delivering a payload of interest to the central nervous system of an individual, the method comprising administering to the individual the recombinant AAV particle of claim 20.

30. The method of claim 29, wherein said administering comprises parenteral administration of the recombinant AAV particle.

31. The variant AAV capsid protein of claim 1, wherein the variant AAV capsid protein comprises the amino acid sequence set forth in any one of SEQ ID NOs: 1-27.

32. The recombinant AAV particle of claim 20, wherein the variant AAV capsid protein comprises the amino acid sequence set forth in any one of SEQ ID NOs: 1-27.


SEQUENCE LISTING (57 sequences)

SEQ ID NO: 1 - protein, 735 aa, Artificial sequence
  Synthetic Sequence
MAADGYLPDWLEDNLSEGIREWWALQPGAPQPKANQQHQDNARGLVLPGYKYLGPGNGLDKGEPVNAADAAALEHDKAYDQQLQAGDNPYLKYNHADAEFQERLQEDTSFGGNLGRAVFQAKKRVLEPFGLVEEGAKTAPGKKRPVEQSPQEPDSSSGIGKTGQQPARKRLNFGQTGDTDSAADPQPLGEPPAAPTSLGSNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSLAGPNNMSAQAKNWLPGPCYRQQRVSTTLSQNNNSNFAWTGATKYHLNGRDSLVNPGVAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Artificial sequence
  Synthetic Sequence
MAADGYLPDWLEDNLSEGIREWWALQPGAPQPKANQQHQDNARGLVLPGYKYLGPGNGLDKGEPVNAADAAALEHDKAYDQQLQAGDNPYLKYNHADAEFQERLQEDTSFGGNLGRAVFQAKKRVLEPFGLVEEGAKTAPGKKRPVEQSPQEPDSSSGIGKTGQQPARKRLNFGQTGDTDSAADPQPLGEPPAAPTSLGSNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEVQYTSNYAKSANVDFTVDNNGLYTEPRPIGTRYLTRPL

SEQ ID NO: 3 - protein, 735 aa, Artificial sequence
  Synthetic Sequence
MAADGYLPDWLEDNLSEGIREWWALQPGAPQPKANQQHQDNARGLVLPGYKYLGPGNGLDKGEPVNAADAAALEHDKAYDQQLQAGDNPYLKYNHADAEFQERLQEDTSFGGNLGRAVFQAKKRVLEPFGLVEEGAKTAPGKKRPVEQSPQEPDSSSGIGKTGQQPARKRLNFGQTGDTDSAADPQPLGEPPAAPTSLGSNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEVQYTSNYAKSANVDFTVDNNGLYTEPRPIGTRYLTRPL

SEQ ID NO: 4 - protein, 737 aa, Artificial sequence
  Synthetic Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRLLEPFGLVEEGAKTAPGKKRPVEQSPQEPDSSSGIGKTGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNDGTTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQSTGGTAGTQQLLFSQAGPNNMSAQAKNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRNL

SEQ ID NO: 5 - protein, 735 aa, Artificial sequence
  Synthetic Sequence
MAADGYLPDWLEDNLSEGIREWWALKPGVPQPKANQQHQDNRRGLVLPGYKYLGPGNGLDKGEPVNEADAAALEHDKAYDQQLKAGDNPYLRYNHADAEFQERLQEDTSFGGNLGRAVFQAKKRVLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYNKSVNVDFTVDTNGVYSEPRPIGTRYLTRNL

SEQ ID NO: 6 - protein, 738 aa, Artificial sequence
  Synthetic Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPAAPTSLGSNTMASGGGAPMADNNEGADGVGNASGNWHCDSTWLGDRVITTSTRTWALPTYNNHLYKQISNSTSGGSSNDNAYFGYSTPWGYFDFNRFHCHFSPRDWQRLINNNWGFRPKRLSFKLFNIQVKEVTDNNGVKTIANNLTSTVQVFSDSEYQLPYVLGSAHQGCLPPFPADVFMVPQYGYLTLNNGSQAMGRSSFYCLEYFPSQMLRTGNNFTFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEIQYTSNYYKSTSVDFAVNTEGVYSEPRPIGTRYLTRNL

SEQ ID NO: 7 - protein, 735 aa, Artificial sequence
  Synthetic Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEHSPVEPDSSSGTGKAGQQPARKRLNFGQTGDTDSAADPQPLGEPPAAPAAVGPTTMASGGGAPMADNNEGADGVGNASGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTNVDFAVNTDGTYSEPRPIGTRYLTRPL

SEQ ID NO: 8 - protein, 735 aa, Artificial sequence
  Synthetic Sequence
MAADGYLPDWLEDTLSEGIRQWWKLKPGPPPPKPAERHKDDSRGLVLPGYKYLGPFNGLDKGEPVNEADAAALEHDKAYDRQLKAGDNPYLRYNHADAEFQERLKEDTSFGGNLGRAVFQAKKRVLEPFGLVEEGAKTAPGKKRPVEQSPQEPDSSSGIGKTGQQPAKKRLNFGQTGDSESVPDPQPIGEPPAGPSGLGSGTMASGSGAPMADNNEGADGVGNASGNWHCDSTWLGDRVITTSTRTWALPTYNNHLYKQISSQSGANNDNHYFGYSTPWGYFDFNRFHCHFSPRDWQRLINNNWGFRPKRLNFKLFNIQVKEVTQTDGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRATNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEVQYTSNYAKSANVDFTVDNNGLYTEPRPIGTRYLTRPL

SEQ ID NO: 9 - protein, 736 aa, Artificial sequence
  Synthetic Sequence
MAADGYLPDWLEDNLSEGIREWWDLKPGAPKPKANQQHQDNARGLVLPGYKYLGPGNGLDKGEPVNAADAAALEHDKAYDQQLQAGDNPYLKYNHADAEFQERLQEDTSFGGNLGRAVFQAKKRVLEPLGLVEEGAKTAPGKKRPVEQSPQEPDSSTGIGKKGQQPARKRLNFGQTGDSESVPDPQPIGEPPAAPTSLGSNTMAAGGGAPMADNNEGADGVGNSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTIQVFTDSEYQLPYVLGSAHQGCFPPFPADVFMVPQYGYLTLNNGSQAMGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQNRDVYLQGPIWAKIPHTDGNFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 10 - protein, 735 aa, Artificial sequence
  Synthetic Sequence
MAADGYLPDWLEDNLSEGIREWWDLKPGAPKPKANQQHQDNRRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGANNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHVMGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTSVDFAVNTEGVYSEPRPIGTRYLTRNL

SEQ ID NO: 11 - protein, 736 aa, Artificial sequence
  Synthetic Sequence
MAADGYLPDWLEDNLSEGIREWWALKPGAPKPKANQQKQDDGRGLVLPGYKYLGPGNGLDKGEPVNAADAAALEHDKAYDQQLQAGDNPYLRYNHADAEFQERLQEDTSFGGNLGRAVFQAKKRVLEPLGLVEEAAKTAPGKKRPVDQSPQEPDSSSGVGKSGKQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKNPPPQILIKNTPVPANPPETFNPEKLNSFITQYSTGQVSVEIEWELQKENSKRWNPEIQYTSNYYKSTSVDFAVNTEGVYSEPRPIGTRYLTRNL

SEQ ID NO: 12 - protein, 737 aa, Artificial sequence
  Synthetic Sequence
MAADGYLPDWLEDTLSEGIRQWWKLKPGPPPPKPAERHKDDSRGLVLPGYKYLGPFNGLDKGEPVNEADAAALEHDKAYDRQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPTSLGSNTMASGGGAPVADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKKLSFKLFNIQVKEVTTNDGVTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 13 - protein, 736 aa, Artificial sequence
  Synthetic Sequence
MAADGYLPDWLEDNLSEGIREWWDLKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPFGLVEEAAKTAPGKKRPVEHSPVEPDSSSGTGKAGQQPARKRLNFGQTGDADSVPDPQPLGQPPAAPSGLGTNTMAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMVPQYGYLTLNNGSQAVGRSSFYCLEYFPSQMLRTGNNFEFSYQFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYNKSVNVDFTVDTNGVYSEPRPIGTRYLTRPL

SEQ ID NO: 14 - protein, 736 aa, Artificial sequence
  Synthetic Sequence
MAADGYLPDWLEDNLSEGIREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVDQSPQEPDSSSGVGKSGKQPARKRLNFGQTGDSESVPDPQPLGEPPAAPTSLGSNTMASGGGAPVADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGNFHPSPLMGGFGMKHPPPQILIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 15 - protein, 737 aa, Artificial sequence
  Synthetic Sequence
MAADGYLPDWLEDTLSEGIRQWWKLKPGPPPPKPAERHKDDSRGLVLPGYKYLGPFNGLDKGEPVNAADAAALEHDKAYDQQLKAGDNPYLRYNHADAEFQERLKEDTSFGGNLGRAVFQAKKRLLEPLGLVEEAAKTAPGKKRPVEQSPQEPDSSAGIGKSGAQPAKKRLNFGQTGDSESVPDPQPLGEPPAGPSGLGSGTMAAGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGNFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 16 - protein, 736 aa, Artificial sequence
  Synthetic Sequence
MAADGYLPDWLEDNLSEGIREWWALQPGAPKPKANQQHQDNARGLVLPGYKYLGPGNGLDKGEPVNAADAAALEHDKAYDQQLKAGDNPYLRYNHADAEFQERLQEDTSFGGNLGRAVFQAKKRLLEPLGLVEEPVKTAPAKKRPIEKSPAEPSSSKGIGKAGQQPARKRLNFGQTGDADSVPDPQPLGQPPAAPTSLGPTTMASGSGAPMADNNEGADGVGNASGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFADSDYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGMKHPPPQILIKNTPVPANPPTTFNQSKLNSFITQYSTGQVSVEIEWELQKENSKRWNPEVQYTSNYYKSTSVDFTVDTNGVYSEPRPIGTRYLTRNL

SEQ ID NO: 17 - protein, 736 aa, Artificial sequence
  Synthetic Sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRLLEPLGLVEEAAKTAPGKKRPVEQSPQEPDSSSGVGKSGKQPARKRLNFGQTGDSESVPDPQPLGEPPAAPTSLGSNTMASGGGAPMADNNEGADGVGNSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQTDGTKTIANNLTSTVQVFADSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQNRDVYLQGPIWAKIPHTDGNFHPSPLMGGFGLKHPPPQILIKNTPVPADPPTAFNKDKLNSFITQYSTGQVSVEIEWELQKENSKRWNPEIQYTSNYNKSVNVDFTVDTNGVYSEPRPIGTRYLTRNL

SEQ ID NO: 18 - protein, 736 aa, Artificial sequence
  Synthetic Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEQSPVEPDSSSGIGKTGQQPAKKRLNFGQTGDTESVPDPQPLGEPPAAPTSLGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 19 - protein, 736 aa, Artificial sequence
  Synthetic Sequence
MAADGYLPDWLEDNLSEGIREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPTSLGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 20 - protein, 737 aa, Artificial sequence
  Synthetic Sequence
MAADGYLPDWLEDTLSEGIRQWWKLKPGPPPPKPAERHKDDSRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPIGEPPAGPSGLGSGTMAAGGGAPMADNNEGADGVGNSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEEVPFHSSYAHSQSLDRLMNPLIDQYLYYLSRTQSTGGTAGTQQLLFSQAGPNNMSAQAKNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 21 - protein, 737 aa, Artificial sequence
  Synthetic Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPIGEPPAGPSGLGSGTMAAGGGAPMADNNEGADGVGNSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEEVPFHSSYAHSQSLDRLMNPLIDQYLYYLSRTQSTGGTAGTQQLLFSQAGPNNMSAQAKNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 22 - protein, 735 aa, Artificial sequence
  Synthetic Sequence
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEGAKTAPGKKRPVEQSPQEPDSSSGTGKAGQQPARKRLNFGQTGDADSVPDPQPIGEPPAAPSGLGTGTMASGSGAPMADNNEGADGVGNSSGNWHCDSQWLGDRVITTSTRTWALPTYNNHLYKQISSQSGANNDNHYFGYSTPWGYFDFNRFHCHFSPRDWQRLINNNWGFRPKRLNFKLFNIQVKEVTQNEGTKTIANNLTSTIQVFTDSEYQLPYVLGSAHEGCLPPFPADVFMVPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTGASKYNLNGRESIINPGTAMASHKDDEDKFFPMSGVMIFGKESAGASNTALDNVMITDEEEIKATNPVATERFGTVAVNFQSSSTDPATGDVHVMGALPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRPL

SEQ ID NO: 23 - protein, 719 aa, Artificial sequence
  Synthetic Sequence
MAADGYLPDWLEDTLSEGIRQWWKLKPGPPPPKPAERHKDDSRGLVLPGYNYLGPGNGLDRGEPVNEADAAALEHDKAYDQQLKAGDNPYLKYNHADAEFQERLKEDTSFGGNLGRAVFQAKKRVLEPLGLVEEPVKTAAKGERIDDHYPKKKKARIEETEAGTSGAQQLQIPAQPASSLGADTMSAGGGGPLGDNNQGADGVGNASGDWHCDSTWMGDRVVTKSTRTWALPTYNNHLYKQISSASTGASNDNHYFGYSTPWGYFDFSRFHCHFSPRDWQRLINNNWGFRPKRLSFKLFNIQVKEVTTQDGTTTIANNLTSTVQVFTDNDYQLPYILGSAHQGCLPPFPADVFMVPQYGYLTLNNGSQAVGRSSFYCPEYFPSQMLRTGNNFQFTYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTATSKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYYKSTSVDFAVNTEGVYSEPRPIGTRYLTRPL

SEQ ID NO: 24 - protein, 735 aa, Artificial sequence
  Synthetic Sequence
MSFVDHPPDWLEEVGEGLHEFLELEAGPPKPKPNQQKQDNARGLVLPGYNYLGPFNGLDKGEPVNEADAAALEHDKAYDQQLKAGDNPYLKYNHADAEFQERLKEDTSFGGNLGRAVFQAKKRVLEPLGLVEEAAKTAPGKKRPVDQSPQEPDSSSGVGKSGKQPARKRLNFGQTGDSESVPDPQPIGEPPAGPSGLGSGTMAAGSGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTQNEGTKTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEVQYTSNYAKSANVDFTVDNNGLYTEPRPIGTRYLTRNL

SEQ ID NO: 25 - protein, 735 aa, Artificial sequence
  Synthetic Sequence
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDSESVPDPQPIGEPPAGPTSLGSNTMASGGGAPMADNNEGADGVGNASGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSDYQLPYVLGSAHQGCLPPFPADVFMVPQYGYLTLNNGSQAVGRSSFYCLEYFPSQMLRTGNNFQFTYTFEDVPFHSSYAHSQSLDRLMNPLIDQYLYYLSRTQSTGGTAGTQQLLFSQAGPNNMSAQAKNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEIQYTSNYNKSVNVDFTVDTNGVYSEPRPIGTRYLTRPL

SEQ ID NO: 26 - protein, 736 aa, Artificial sequence
  Synthetic Sequence
MAADGYLPDWLEDNLSEGIREWWDLKPGAPKPKANQQKQDDGRGLVLPGYKYLGPFNGLDKGEPVNAADAAALEHDKAYDRQLDSGDNPYLKYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPLGEPPAAPTSLGSNTMASGGGAPMADNNEGADGVGNSSGNWHCDSTWLGDRVITTSTRTWALPTYNNHLYKQISSQSGANNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTNPATGDVHAMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 27 - protein, 737 aa, Artificial sequence
  Synthetic Sequence
MAADGYLPDWLEDTLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEHSPVEPDSSSGIGKTGQQPAKKRLNFGQTGDSESVPDPQPLGEPPATPAAVGPTTMASGGGAPMADNNEGADGVGSSSGNWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEVQYTSNYNKSVNVEFTVDNNGVYSEPRTIGTRYLTRNL

SEQ ID NO: 28 - protein, 735 aa, adeno-associated virus 2 [sequence omitted]

SEQ ID NO: 29 - protein, 736 aa, adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 30 - protein, 4 aa, Artificial sequence
  Synthetic Sequence
AAAA

SEQ ID NO: 31 - DNA, 2208 bp, Artificial sequence
  Synthetic Sequence
atggctgccgatggttatcttccagattggctcgaggacaacctctctgagggcattcgcgagtggtgggcgctgcaacctggagcccctcaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctgcaggcgggtgacaatccgtacctcaagtacaaccacgccgacgcggagtttcaggagcgtctgcaagaagatacgtcttttgggggcaacctcgggcgagcagtcttccaggccaagaagagggttctcgaaccttttggtctggttgaggaaggcgctaagacggctcctggaaagaaacgtccggtagagcagtcgccacaagagccagactcctcctcgggcatcggcaagacaggccagcagcctgcgaggaagcgactcaactttggtcagactggagacaccgactccgccgctgacccccagcctctcggagaaccaccagcagcccccacaagtttgggatctaatacaatggcttcaggcggtggcgcaccaatggcagacaataacgagggtgccgacggagtgggtaatgcctcgggaaattggcattgcgattccacatggctgggcgaccgagtcatcaccaccagcacccgaacctgggccctccccacctacaacaaccacctctacaagcaaatctccagccaatcaggagcctcgaacgacaatcactactttggctacagcaccccttgggggtattttgatttcaacagattccactgccacttttcaccacgtgactggcagcgactcatcaacaacaactggggattccgacccaagagactcaacttcaagctctttaacattcaagtcaaagaggtcacgcagaatgacggtacgacgacgattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctctggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttacctcacccgtcccctgtaa

SEQ ID NO: 32 - DNA, 2208 bp, Artificial sequence
  Synthetic Sequence
atggctgccgatggttatcttccagattggctcgaggacaacctctctgagggcattcgcgagtggtgggcgctgcaacctggagcccctcaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctgcaggcgggtgacaatccgtacctcaagtacaaccacgccgacgcggagtttcaggagcgtctgcaagaagatacgtcttttgggggcaacctcgggcgagcagtcttccaggccaagaagagggttctcgaaccttttggtctggttgaggaaggcgctaagacggctcctggaaagaaacgtccggtagagcagtcgccacaagagccagactcctcctcgggcatcggcaagacaggccagcagcctgcgaggaagcgactcaactttggtcagactggagacaccgactccgccgctgacccccagcctctcggagaaccaccagcagcccccacaagtttgggatctaatacaatggcttcaggcggtggcgcaccaatggcagacaataacgagggtgccgacggagtgggtaatgcctcgggaaattggcattgcgattccacatggctgggcgaccgagtcatcaccaccagcacccgaacctgggccctccccacctacaacaaccacctctacaagcaaatctccagccaatcaggagcctcgaacgacaatcactactttggctacagcaccccttgggggtattttgatttcaacagattccactgccacttttcaccacgtgactggcagcgactcatcaacaacaactggggattccgacccaagagactcaacttcaagctctttaacattcaagtcaaagaggtcacgcagaatgacggtacgacgacgattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttacctcacccgtcccctgtaa

SEQ ID NO: 33 - DNA, 2208 bp, Artificial sequence
  Synthetic Sequence
atggctgccgatggttatcttccagattggctcgaggacaacctctctgagggcattcgcgagtggtgggcgctgcaacctggagcccctcaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctgcaggcgggtgacaatccgtacctcaagtacaaccacgccgacgcggagtttcaggagcgtctgcaagaagatacgtcttttgggggcaacctcgggcgagcagtcttccaggccaagaagagggttctcgaaccttttggtctggttgaggaaggcgctaagacggctcctggaaagaaacgtccggtagagcagtcgccacaagagccagactcctcctcgggcatcggcaagacaggccagcagcctgcgaggaagcgactcaactttggtcagactggagacaccgactccgccgctgacccccagcctctcggagaaccaccagcagcccccacaagtttgggatctaatacaatggcttcaggcggtggcgcaccaatggcagacaataacgagggtgccgacggagtgggtaatgcctcgggaaattggcattgcgattccacatggctgggcgaccgagtcatcaccaccagcacccgaacctgggccctccccacctacaacaaccacctctacaagcaaatctccagccaatcaggagcctcgaacgacaatcactactttggctacagcaccccttgggggtattttgatttcaacagattccactgccacttttcaccacgtgactggcagcgactcatcaacaacaactggggattccgacccaagagactcaacttcaagctctttaacattcaagtcaaagaggtcacgcagaatgacggtacgacgacgattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttacctcacccgtcccctgtaa

SEQ ID NO: 34 - DNA, 2214 bp, Artificial sequence
  Synthetic Sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaaccttttggtctggttgaggaaggtgctaagacggctcctggaaagaaacgtccggtagagcagtcgccacaagagccagactcctcctcgggcattggcaagacaggccagcagcccgccagaaaaagactcaattttggtcagactggcgactcagagtcagttccagaccctcaacctctcggagaacctccagcagcgccctctggtgtgggacctaatacaatggctgcaggcggtggcgctccaatggcagacaataacgaaggcgccgacggagtgggtagttcctcaggaaattggcattgcgattccacatggctgggcgacagagtcatcaccaccagcaccagaacctgggccctgcccacttacaacaaccatctctacaagcaaatctccaacagcacatctggaggatcttcaaatgacaacgcctacttcggctacagcaccccttgggggtattttgacttcaaccgcttccactgccacttctccccgcgagactggcagcggctcatcaacaacaactgggggttccggcccaagcgactcaacttcaagctcttcaacatccaagttaaagaggtcacgcagaacgatggcacgacgactattgccaataaccttaccagcacggttcaagtcttctcggactcggagtaccagcttccgtacgtcctcggctctgcgcaccagggctgcctccctccgttcccggcggacgtgttcatgattccccagtacgggtatctgacgctcaacaatggcagccaagccgtgggacgttcatccttttactgcctggaatatttcccatcgcagatgctgagaacgggcaataactttaccttcagctacaccttcgaggacgttcctttccacagcagctacgcgcacagccagagtctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtacacatccaattatgcaaaatctgccaacgttgattttactgtggacaacaatggactttatactgagcctcgccccattggcacccgttacctcacccgtaatctgtaa

SEQ ID NO: 35 - DNA, 2208 bp, Artificial sequence
  Synthetic Sequence
atggctgctgacggttatcttccagattggctcgaggacaacctttctgaaggcattcgcgagtggtgggctttgaaacctggagtccctcaacccaaagcgaaccaacaacaccaggacaaccgtcggggtcttgtgcttccgggttacaaataccttggacccggcaacggactcgacaagggagagccggtcaacgaggcagacgccgcggccctcgaacacgacaaagcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctcttggtctggttgaggaagcggctaagacggctcctggaaagaagaggcctgtagatcagtctcctcaggaaccggactcatcatctggtgttggcaaatcgggcaaacagcccgccagaaaaagactcaattttggtcagactggcgactcagagtcagttccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcacccgaacatgggccttgcccacctataacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagagactcaacttcaaactcttcaacatccaagtcaaggaggtcacgcagaatgaaggcaccaagaccatcgccaataacctcaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggacggcaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaggaaaacagcaagcgctggaatcccgaaattcagtacacttccaactacaacaagtctgttaatgtggactttactgtagacactaatggtgtttatagtgaacctcgccccattggcacccgttacctcacccgtaatctgtaa

SEQ ID NO: 36 - DNA, 2217 bp, Artificial sequence
  Synthetic Sequence
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaaggcgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaaccaccagcagcccccacaagtttgggatctaatacaatggcttcaggcggtggcgcaccaatggcagacaataacgagggcgccgacggagtgggtaatgcctcgggaaattggcattgcgattccacatggctgggcgaccgagtcatcaccaccagcacccgcacctgggccctgcccacctacaataaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacattcaggtcaaagaggttacggacaacaatggagtcaagaccatcgccaataaccttaccagcacggttcaagtcttctcggactcggagtaccagctcccgtacgtcctcggctcggcgcatcaaggatgcctcccgccgttcccagcagacgtcttcatggtcccgcagtacgggtatttgacgctgaacaacggcagccaggcgatgggtcgctcgtccttctactgcctggagtactttccgtcgcagatgctgcgtaccggaaacaactttaccttcagctacacttttgaggacgttcctttccacagcagctacgcccacagccagagcctggaccggctgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtacctgcagggtcccatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 37 - DNA, 2208 bp, Artificial sequence
  Synthetic Sequence
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcgaggaagcgactcaactttggtcagactggagacaccgactccgccgctgacccccagcctctcggagaaccaccagcagcccccgctgctgtgggacctactacaatggcttcaggcggtggcgcaccaatggcagacaataacgaaggcgccgacggagtgggtaatgcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttttcaccacgtgactggcagcggctcatcaacaacaactggggattccggcccaagagactcaacttcaagctcttcaacatccaagttaaagaggtcacgcagaacgatggcacgacgactattgccaataaccttaccagcacggttcaagtgtttacggactcggagtatcagctcccgtacgtgctcggctctgcgcaccagggctgcctgcctccgttcccggcggacgtgttcatgattccgcagtacggctacctaacgctcaacaatggcagccaggcagtgggacggtcatccttttactgcctggaatatttcccatcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcagtccacgggaggtaccgcaggaactcagcagttgctattttctcaggccgggcctaataacatgtcggctcaggccaaaaactggctacctggaccctgttatcggcagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacaccccggtacctgcgaatccttcgaccaccttcagtgcggcaaagtttgcttccttcatcacacagtactccacgggacaggtcagcgtggagatcgagtgggagctgcagaaagaaaacagcaaacgctggaacccagagattcaatacacttccaactactacaaatctacaaatgtggactttgctgttaacacagatggcacttattctgaacctcgccccattggcacccgttaccttacccgtcccctgtaa

SEQ ID NO: 38 - DNA, 2208 bp, Artificial sequence
  Synthetic Sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcaaagcgggtgacaatccgtacctgcggtataaccacgccgacgccgagttccaggagcgactcaaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaactttgggcagactggcgactcagagtcagtgcccgaccctcaaccaatcggagaaccccccgcaggcccctctggtctgggatctggtacgatggcttcaggcagtggcgcaccaatggcagacaataacgaaggcgccgacggagtgggtaatgcctcaggaaattggcattgcgattccacatggctgggcgacagagtcatcaccaccagcacccgaacctgggccctgcccacttacaacaaccacctctacaagcaaatctccagccagtctggagccaacaacgacaaccactactttggctacagcaccccctgggggtactttgacttcaaccgcttccactgccacttctccccgcgagactggcagcggctcatcaacaacaactgggggttccggcccaagcgactcaacttcaagctcttcaacatccaagtcaaggaggtcactcagacggacggcacgaagaccatcgccaataaccttaccagcacgattcaggtctttacggactcggaataccagctcccgtacgtcctcggctctgcgcaccagggctgcctccctccgttcccggcggacgtgttcatgattccgcagtacggctacctaacgctcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctcatcgaccagtacctgtattacttgagcagaacaaacactccaagtggaaccaccacgcagtcaaggcttcagttttctcaggccggagcgagtgacattcgggaccagtctaggaactggctacctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctatgcacggcaatctaatatttggcaaagaagggacaacggcaagtaacgcagaattagataatgtaatgattacggatgaagaagagattcgtgccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttccttcatcacgcaatacagcaccggacaggtcagcgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttacctcacccgtcccctgtaa

SEQ ID NO: 39 - DNA, 2211 bp, Artificial sequence
  Synthetic Sequence
atggctgctgacggttatcttccagattggctcgaggacaacctttctgaaggcattcgagagtggtgggacttgaaacctggagccccgaagcccaaagccaaccaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctgcaggcgggtgacaacccctacctcaagtacaaccacgccgacgccgagttccaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcgccacaagagccagactcctctacgggcatcggcaagaaaggccaacagcccgccagaaaaagactcaattttggtcagactggcgactcagagtcagttccagaccctcaaccaatcggagaaccaccagcagcccccacaagtttgggatctaatacaatggctgcaggcggtggcgcaccaatggcagacaataacgagggcgccgacggagtgggtaattcctcgggaaattggcattgcgattcccaatggctgggcgacagagtcatcaccaccagcaccagaacctgggccctgcccacctacaataaccacctctacaagcaaatctccagtgcttcaacgggggccagcaacgacaaccactactttggctacagcaccccttgggggtattttgactttaacagattccactgccacttctcaccacgtgactggcagcgactcatcaacaacaactgggggttccggcccaagcgactcaacttcaagctcttcaacattcaggtcaaagaggttacggacaacaatggagtcaagaccatcgccaataaccttaccagcacgattcaggtctttacggactcggaataccagctcccgtacgtcctcggctctgcgcaccagggctgcttcccgccgttcccggcggacgtcttcatggtcccgcagtacgggtatttgacgctgaacaacggcagccaggcgatgggtcgctcgtccttctactgcctggagtactttccgtcgcagatgctgagaacgggcaataacttccaattcagctataccttcgaggatgtaccttttcacagcagctacgcgcacagccagagcttggaccggctgatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacccggtatggtctggcagaaccgggacgtgtacctgcagggtcctatctgggccaagattcctcacacggacggaaactttcatccctcgccgctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 40 - DNA, 2208 bp, Artificial sequence
  Synthetic Sequence
atggctgccgatggttatcttccagattggctcgaggacaacctttctgaaggcattcgtgagtggtgggacttgaaacctggagccccgaagcccaaagcgaaccaacaacaccaggacaaccgtcggggtcttgtgcttccgggttacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgcggagtttcaggagcgtctgcaagaagatacgtcttttgggggcaacctcgggcgagcagtcttccaggccaagaagcgggttctcgaacctctcggtctggttgaggaagcggctaagacggctcctggaaagaagaggcctgtagagcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacctacaacaaccacctctacaagcaaatctccagccagtctggagccaacaacgacaaccactactttggctacagcaccccctgggggtactttgacttcaaccgcttccactgccatttctcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgggggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccccgtggccaccgaaagatttgggactgtggcagtcaatttccagagcagcagcacagaccctgcgaccggagatgtgcatgttatgggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaagtgtggactttgctgttaatacagaaggcgtgtactctgaaccccgccccattggcacccgttacctcacccgtaatctgtaa

SEQ ID NO: 41 - DNA, 2211 bp, Artificial sequence
  Synthetic Sequence
atggctgccgatggttatcttccagattggctcgaggacaacctctctgagggcattcgcgagtggtgggcgctgaaacctggagccccgaagcccaaagccaaccagcaaaagcaggacgacggccggggtcttgtgcttccgggttacaaatacctcggacccggtaacggactcgacaaaggagagccggtcaacgcggcggatgcagcggccctcgagcacgacaaggcctacgaccagcagctgcaggcgggtgacaatccgtaccttcggtataaccacgccgacgccgagtttcaggagcgtctgcaagaagatacgtcttttgggggcaacctcgggcgagcagtcttccaggccaagaagcgggttctcgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtgcccgaccctcaaccaatcggagaaccccccgcaggcccctctggtctgggatctggtacaatggctgcaggcggtggcgcaccaatggcagacaataacgagggcgccgacggagtgggtagttcctcgggaaattggcattgcgattccacatggctgggggacagagtcatcaccaccagcaccagaacctgggccctgcccacttacaacaaccatctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagaaactcagcttcaagctcttcaacatccaagttaaagaggtcacgcagaacgatggcacgacgactattgccaataaccttaccagcacggttcaagtgtttacggactcggaataccagctcccgtacgtcctcggctctgcgcaccagggctgcctgcctccgttcccggcggacgtgttcatgattccccagtacggctacctaacactcaacaatggcagccaggcagtgggacggtcatccttttactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggaaactttcatccctcgccgctgatgggaggctttggactcaagaacccgcctcctcagatcctcatcaaaaacacgcctgttcctgcgaatcctccagagacgttcaacccggaaaagctcaattctttcataactcaatattctacgggccaggtcagcgtggagatcgagtgggagctgcagaaggagaacagcaagcgctggaaccccgagattcagtacacctccaactactacaaatctacaagtgtggactttgctgttaatacagaaggcgtgtactctgaaccccgccccattggcacccgttacctcacccgtaatctgtaa

SEQ ID NO: 42 - DNA, 2214 bp, Artificial sequence
  Synthetic Sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagccatcaccccagcgttctccagactcctctacgggcatcggcaagaaaggccaacagcccgccagaaaaagactaaatttcggtcagactggcgactcagagtcagttccagaccctcaacctctcggagaaccaccagcagcccccacaagtttgggatctaatacaatggcttcaggtggtggcgcaccagtggcagacaataacgaaggtgccgatggagtgggtagttcctcgggaaattggcattgcgattccacatggctgggcgaccgagtcatcaccaccagcacccgaacatgggccttgcccacctacaataaccacctctacaagcaaatctccagtgcttcaacgggggccagcaacgacaaccactactttggctacagcaccccttgggggtattttgacttcaacagattccactgccacttctcaccacgtgactggcagcgactcattaacaacaactggggattccggcccaagaaactcagcttcaaactcttcaacatccaagtcaaggaggtcacgacgaatgatggcgtcacaaccatcgctaataaccttaccagcacggttcaagtgtttacggactcggagtatcagctcccgtacgtcctcggctcggcgcatcaaggatgcctccctccgttcccggcggacgtgttcatgattccgcaatacggctacctgacgctcaacaatggcagccaagccgtgggacgttcatccttttactgcctggagtacttcccttcgcagatgctaaggactggaaataacttccaattcagctataccttcgaggatgtaccttttcacagcagctacgctcacagccagagtttggaccggctgatgaatcctctgattgaccagtacctgtattatttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttacctcacccgtaatctgtaa

SEQ ID NO: 43 - DNA, 2211 bp, Artificial sequence
  Synthetic Sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctgcaggcggtggcgcaccaatggcagacaataacgaaggcgccgacggagtgggtagttcctcgggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccttgggggtattttgactttaacagattccactgccacttttcaccacgtgactggcagcgactcattaacaacaactggggattccggcccaagaaactcagcttcaagctcttcaacatccaggtcaaggaggtcacgcagaatgaaggcaccaagaccatcgccaataaccttaccagcacgattcaggtctttacggactcggagtatcagctcccgtacgtgctcgggtcggcgcaccaaggctgtctcccgccgtttccagcggacgtcttcatggtgccacagtatggatacctcaccctgaacaacggaagtcaagcggtgggacgctcatccttctactgcctggagtactttccttctcaaatgctgagaacgggcaacaactttgagttcagctaccagtttgaggacgtgcctttccacagcagctacgcccacagccagagcttggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggacttaagcacccgcctcctcagatcctgatcaagaacacgcctgtacctgcggatcctccgaccaccttcaaccagtcaaagctgaactctttcatcacgcaatacagcaccggacaggtcagcgtggaaattgagtgggagctgcagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 44 - DNA, 2211 bp, Artificial sequence
  Synthetic Sequence
atggctgccgatggttatcttccagattggctcgaggacaacctttctgaaggcattcgagagtggtgggcgctgcaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtaccttcggtataaccacgccgacgccgagtttcaggagcgtctgcaagaagatacgtcttttgggggcaaccttggcagagcagtcttccaggccaagaagagggttctcgaacctctcggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggtggtggcgcaccagtggcagacaataacgaaggtgccgatggagtgggtaattcctcgggaaattggcattgcgattccacatggatgggcgacagagtcatcaccaccagcacccgaacctgggccctgcccacctacaacaaccacctctacaagcaaatctccagtgcttcaacgggggccagcaacgacaaccactacttcggctacagcaccccctgggggtattttgacttcaacagattccactgccacttctcaccacgtgactggcagcgactcattaacaacaactggggattccggcccaagaaactcagcttcaagctctttaacattcaagtcaaagaggtcacgcagaatgacggtacgacgacgattgccaataaccttaccagcacggttcaggtgtttactgactcggagtaccagttgccgtacgtcctcggctctgcgcaccagggctgcctccctccgttcccggcggacgtcttcatgattcctcagtacgggtacctgactctgaacaatggcagtcaggccgtgggccgttcctccttctactgcctggagtactttccttcgcagatgctgagaaccggcaacaacttccagtttacttacaccttcgaggacgtgcctttccacagcagctacgcccacagccagagcttggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaagattcctcacacggacggaaactttcatccctcgccgctgatgggagggtttggaatgaaacacccgcctcctcagatcctcatcaaaaacacgcctgttcctgcgaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtggacactaatggcgtgtattcagagcctcgccccattggcacccgttaccttacccgtcccctgtaa

SEQ ID NO: 45 - DNA, 2214 bp, Artificial sequence
  Synthetic Sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagccggtcaacgcagcagacgcggcggccctcgagcacgacaaggcctacgaccagcagctcaaggccggtgacaatccgtaccttcggtataaccacgccgacgccgagttccaggagcggctcaaagaagatacgtcttttgggggcaacctcgggcgagcagtcttccaggccaaaaagaggcttcttgaacctcttggtctggttgaggaagcagctaaaacggctcctggaaagaagaggcctgtagagcagtctcctcaggaaccggactcctccgcgggtattggcaaatcgggtgcacagcccgctaaaaagagactcaatttcggtcagactggcgactcagagtcagtccccgacccacaacctctcggagaaccccccgcaggcccctctggtctgggatctggtacaatggctgcaggcggtggcgctccaatggcagacaataacgaaggcgccgacggagtgggtaatgcctcaggaaattggcattgcgattccacatggctgggcgacagagtcatcaccaccagcacccgcacctgggccctgcccacctacaacaaccacctctacaagcaaatctccagtgcttcaacgggggccagcaacgacaaccactacttcggctacagcaccccctgggggtattttgatttcaacagattccactgccacttctcaccacgtgactggcagcgactcatcaacaacaattggggattccgacccaagagactcaacttcaagctctttaacattcaagtcaaagaggtcacgacgaatgatggcgtcacgaccatcgccaataaccttaccagcacggtccaggtcttcacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggccaagattcctcacacggacggaaactttcatccctcgccgctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaaccccgccccattggcacccgttacctcacccgtaatctgtaa

SEQ ID NO: 46 - DNA, 2211 bp, Artificial sequence
  Synthetic Sequence
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaacctgttaagacggctccagccaagaagcggccgatagagaagtctccggcggaaccgagctcttcgaagggcatcggaaaggcgggccagcagcctgcaagaaaaagattgaattttggtcagactggagacgcagactcagtacctgacccccagcctctcggacagccaccagcagcccccacaagtttgggacctactacaatggcttcaggcagtggcgcaccaatggcagacaataacgaaggcgccgacggagtgggtaatgcctcgggaaattggcattgcgattcccaatggctgggcgacagagtcatcaccaccagcacccgcacctgggccttgcccacctacaataaccacctctacaagcaaatctccagccaatcaggagcttcaaacgacaaccactactttggctacagcaccccttgggggtattttgactttaacagattccactgccacttttcaccacgtgactggcagcgactcatcaacaacaactggggattccgacccaagagactcaacttcaagctcttcaacatccaagtcaaggaggtcacgacgaatgatggcgtcacgaccatcgccaataaccttaccagcacggttcaggtctttgcggactcagactatcagctcccgtacgtgctcgggtcggcgcaccagggctgcctgcctccgttcccagcggacgttttcatgattccgcagtacggctacctaacgctcaacaatggcagccaagccgtgggacgttcatccttttactgcctggaatatttcccttctcagatgctgagaacgggcaataactttaccttcagctacaccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctccgctgatgggagggtttggaatgaaacacccgcctcctcagatcctcatcaaaaacacgcctgttcctgcgaatcctccgaccaccttcaaccagtcaaagctgaactctttcatcacgcaatacagcaccggacaggtcagcgtggaaattgaatgggagctgcagaaggaaaacagcaagcgctggaaccccgaggtccagtacacgtccaactactacaaatctacaagtgtggactttactgtggacactaatggcgtgtattcagagcctcgccccattggcaccagatacctgactcgtaatctgtaa

SEQ ID NO: 47 - DNA, 2211 bp, Artificial sequence
  Synthetic Sequence
atggctgccgatggttatcttccagattggctcgaggacaacctctctgagggcattcgcgagtggtgggctctgaaacctggagccccgaaacccaaagccaaccagcaaaagcaggacgacggccggggtctggtgcttcctggctacaagtacctcggacccttcaacggactcgacaagggggagccggtcaacgcagcagacgcggcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaaaaagaggcttcttgaacctctcggtctggttgaggaagcggctaagacggctcctggaaagaagaggcctgtagagcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggcgccgatggagtgggtaattcctcaggaaattggcattgcgattccacatggctgggcgacagagtcatcaccaccagcacccgaacatgggccttgcccacctacaataaccacctctacaagcaaatctccagtgcttcaacgggggccagcaacgacaaccactacttcggctacagcaccccctgggggtattttgacttcaacagattccactgccacttttcaccacgtgactggcagcgactcatcaacaacaactggggattccggcccaagagactcaacttcaaactcttcaacatccaggtcaaggaggtcactcagacggacggcacgaagaccatcgccaataaccttaccagcacggttcaggtctttgcggactcggagtaccagctcccgtacgtcctcggctcggcgcatcaaggatgcctcccgccgttcccagcagacgtcttcatggtgccacagtatggatacctcaccctgaacaacggaagtcaagcggtgggacgctcatccttttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtctggcagaaccgggacgtgtacctgcagggtcccatctgggccaagattcctcacacggacggaaactttcatccctcgccgctgatgggaggctttggactgaaacatccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactacaacaagtctgttaatgtggactttactgtagacactaatggtgtttatagtgaacctcgccctattggaacccggtatctcacacgaaacttgtaa

SEQ ID NO: 48 - DNA, 2211 bp, Artificial sequence
  Synthetic Sequence
atggctgccgatggttatcttccagattggctcgaggacaaccttagtgaaggaattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctgtggagccagactcctcctcgggcattggcaagacaggccagcagcccgctaaaaagagactcaattttggtcagactggcgacacagagtcagttccagaccctcaacctctcggagaacctccagcagcgcccacaagtttgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaagctcttcaacatccaggtcaaggaggtcacgcagaatgaaggcaccaagaccatcgccaataacctcaccagcaccatccaggtgtttacggactcggagtaccagcttccgtacgtcctcggctctgcgcaccagggctgcctccctccgttcccggcggacgtgttcatgattccccagtacggctacctaacactcaacaacggtagtcaggccgtgggacgctcctccttctactgcctggaatatttcccttctcagatgctgagaacgggcaataactttaccttcagctacaccttcgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcaccagatacctgactcgtaatctgtaa

SEQ ID NO: 49 - DNA, 2211 bp, Artificial sequence
  Synthetic Sequence
atggctgctgacggttatcttccagattggctcgaggacaacctttctgaaggcattcgagagtggtgggcgctgcaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgacacagagtcagttccagaccctcaacctctcggagaacctccagcagcgcccacaagtttgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaagctcttcaacatccaggtcaaggaggtcacgcagaatgaaggcaccaagaccatcgccaataacctcaccagcaccatccaggtgtttacggactcggagtaccagcttccgtacgtcctcggctctgcgcaccagggctgcctccctccgttcccggcggacgtgttcatgattccccagtacggctacctaacactcaacaacggtagtcaggccgtgggacgctcctccttctactgcctggaatatttcccttctcagatgctgagaacgggcaataactttaccttcagctacaccttcgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcaccagatacctgactcgtaatctgtaa

SEQ ID NO: 50 - DNA, 2214 bp, Artificial sequence
  Synthetic Sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcggacgagcagtcttccaggcgaaaaagagggttcttgaacctctgggcctggttgaggaaggcgctaagacggctcctggaaagaaacgtccggtagagcagtcgccacaagagccagactcctcctcgggcattggcaagacaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgagggtgccgatggagtgggtaattcctcaggaaattggcattgcgattcccaatggctgggcgacagagtcatcaccaccagcacccgaacctgggccctgcccacttacaacaaccacctctacaagcaaatctccaacagcacatctggaggatcttcaaatgacaacgcctacttcggctacagcaccccctgggggtattttgactttaacagattccactgccatttctcaccacgtgactggcagcgactcatcaacaacaactgggggttccggcccaagcgactcaacttcaagctcttcaacatccaagtcaaggaggtcacgcagaatgaaggcaccaagaccatcgccaataaccttaccagcacgattcaggtctttacggactcggagtaccagctgccgtacgttctcggctctgcccaccagggctgcctgcctccgttcccggcggacgtgttcatgattccgcaatacggctacctgacgctcaacaatggcagccaagccgtgggacgttcatccttttactgcctggaatatttcccttctcagatgctgagaaccggcaacaacttccagtttacttacacctttgaggaagtgcctttccacagcagctacgcgcacagccagagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggcggctttggacttaagcacccgcctcctcagatcctcatcaaaaacacgcctgttcctgcgaatcctccggcggagttttcagctacaaagtttgcttcattcatcacgcaatacagcaccggacaggtcagcgtggagatcgagtgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 51 - DNA, 2214 bp, Artificial sequence
  Synthetic Sequence
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgagggtgccgatggagtgggtaattcctcaggaaattggcattgcgattcccaatggctgggcgacagagtcatcaccaccagcacccgaacctgggccctgcccacttacaacaaccacctctacaagcaaatctccaacagcacatctggaggatcttcaaatgacaacgcctacttcggctacagcaccccctgggggtattttgactttaacagattccactgccatttctcaccacgtgactggcagcgactcatcaacaacaactgggggttccggcccaagcgactcaacttcaagctcttcaacatccaagtcaaggaggtcacgcagaatgaaggcaccaagaccatcgccaataaccttaccagcacgattcaggtctttacggactcggagtaccagctgccgtacgttctcggctctgcccaccagggctgcctgcctccgttcccggcggacgtgttcatgattccgcaatacggctacctgacgctcaacaatggcagccaagccgtgggacgttcatccttttactgcctggaatatttcccttctcagatgctgagaaccggcaacaacttccagtttacttacacctttgaggaagtgcctttccacagcagctacgcgcacagccagagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggcggctttggacttaagcacccgcctcctcagatcctcatcaaaaacacgcctgttcctgcgaatcctccggcggagttttcagctacaaagtttgcttcattcatcacgcaatacagcaccggacaggtcagcgtggagatcgagtgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 52 - DNA, 2208 bp, Artificial sequence
  Synthetic Sequence
atggctgctgacggttatcttccagattggctcgaggacaacctctctgagggcattcgcgagtggtgggcgctgcaacctggagcccctaaacccaaggcaaatcaacaacatcaggacaacgctcgaggtcttgtgcttccgggttacaaatacctcggacccggtaacggactcgacaagggggagcccgtcaacgcagcagacgcggcagccctcgaacacgacaaagcctacgaccagcagctcaaggccggtgacaacccgtacctcaagtacaaccacgccgacgccgagttccaggagcggctcaaagaagatacgtcttttgggggcaacctcggacgagcagtcttccaggcgaaaaagaggcttcttgaacctctcggtctggttgaggaaggcgctaagacggctcctggaaagaaacgtccggtagagcagtcgccacaagagccagactcctcctcgggaaccggaaaggcgggccagcagcctgcaagaaaaagattgaattttggtcagactggagacgcagactcagtcccagaccctcaaccaatcggagaacctcccgcagccccctctggtctgggaactggtacgatggcttcaggcagtggcgcaccaatggcagacaataacgaaggcgccgacggagtgggtaattcctcgggaaattggcattgcgattcccaatggctgggcgaccgagtcatcaccaccagcacccgaacctgggccctgcccacctacaacaaccatctctacaagcaaatctccagccagtctggagccaacaacgacaaccactactttggctacagcaccccctgggggtattttgatttcaacagattccactgccatttctcaccacgtgactggcagcgactcatcaacaacaactggggattccggcccaagagactcaacttcaaactcttcaacatccaagtcaaggaggtcacgcagaatgaaggcaccaagaccatcgccaataacctcaccagcaccatccaggtgtttacggactcggagtatcagctcccgtacgtgctcgggtcggctcacgagggctgcctcccgccgttcccagcggacgtcttcatggtccctcagtatggatacctcaccctgaacaacggaagtcaagcggtgggacgctcatccttttactgcctggagtacttcccttctcagatgctgcggacggggaacaacttcacgttcagctacaccttcgaggacgtgcccttccacagcagctacgcgcacagccagagtctggaccggctgatgaaccccctcatcgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactggtgcttcaaaatataaccttaatgggcgtgaatctataatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtcccctgtaa

SEQ ID NO: 53 - DNA, 2160 bp, Artificial sequence
  Synthetic Sequence
atggctgccgatggttatcttccagattggctcgaggacactctctctgaaggaataagacagtggtggaagctcaaacctggcccaccaccaccaaagcccgcagagcggcataaggacgacagcaggggtcttgtgctgcctggttataactatctcggacccggaaacggtctcgatcgaggagagccggtcaacgaggcagacgccgcggccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaagaagcgggttctcgaacctctcggtctggttgaggagcctgttaagacggctgctaaaggcgagcggatagacgaccactatcccaaaaagaagaaggctcggatcgaagagaccgaagctggaaccagcggagcccagcagctgcagatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgcacctgggccttgcccacctacaataaccacctctacaagcaaatctccagtgcttcaacgggggccagcaacgacaaccactacttcggctacagcaccccctgggggtattttgatttcagcagattccactgccacttctcaccacgtgactggcagcgactcatcaacaacaactggggattccggcccaagagactcagcttcaagctcttcaacatccaagtcaaggaggtcacgacgcaggacggcaccaccaccatcgccaacaacctcacctccaccgtccaagtgtttacggacaacgactaccagctaccatacatcctcggctcggcgcatcaaggatgcctcccgccgttcccagcagacgtcttcatggtgccacagtatggatacctcaccctgaacaacggaagtcaagcggtgggacgctcatccttttactgcccggaatactttccttcgcagatgctgagaaccggcaacaacttccagtttacttacaccttcgaggacgtgcctttccacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcgaccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcacgcagtacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttaccttacccgtcccctgtaa

SEQ ID NO: 54 - DNA, 2208 bp, Artificial sequence
  Synthetic Sequence
atgtcgtttgttgatcaccctccagattggttagaagaagttggtgaaggccttcacgagtttttggagctcgaagctggcccacccaaaccgaagcccaaccagcagaagcaggacaacgcccgtggtcttgtactgcctggatataattatctgggacccttcaacggactcgacaagggagagccggtcaacgaggcagacgccgcggccctcgagcacgacaaggcctacgaccagcagctcaaggccggagacaacccgtacctcaagtacaaccacgccgacgcggagtttcaggagcgccttaaagaagatacgtcttttgggggcaacctcgggcgagcagtcttccaggccaagaagcgggttctcgaacctctcggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtgcccgaccctcaaccaatcggagaaccccccgcaggcccctctggtctgggatctggtacaatggctgcaggcagtggcgcaccaatggcagacaataacgaaggcgccgacggagtgggtaatgcctcgggaaattggcattgcgattccacatggctgggcgaccgagtcatcaccaccagcacccgcacctgggccctgcccacctacaacaaccatctctacaagcaaatttccagccaatcaggagcctcgaacgacaaccactacttcggctacagcaccccctgggggtattttgactttaacagattccactgccacttctcaccacgtgactggcagcgactcatcaacaacaactgggggttccggcccaagcgactcaacttcaagctcttcaacatccaagtcaaggaggtcactcagaatgaaggcaccaagaccatcgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgctggaatcccgaagtgcagtatacatctaactatgcaaaatctgccaacgttgatttcactgtggacaacaatggactttatactgaaccccgccccattggcacccgttacctcacccgtaatctgtaa

SEQ ID NO: 55 - DNA, 2208 bp, Artificial sequence
  Synthetic Sequence
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggagcccgtcaacgcggcggacgcagcggccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaactttgggcagactggcgactcagagtcagtgcccgaccctcaaccaatcggagaaccccccgcaggccccacaagtttgggatctaatacaatggcttcaggcggtggcgcaccaatggcagacaataacgaaggcgccgacggagtgggtaatgcctcaggaaattggcattgcgattccacatggatgggcgacagagtcatcaccaccagcacccgaacctgggccctgcccacctacaacaaccacctctacaaacaaatttccagccaatcaggagcctcgaacgacaaccactactttggctacagcaccccttgggggtattttgactttaacagattccactgccacttctcaccacgtgactggcagcgactcatcaacaacaactggggattccggcctaagcgactcaacttcaagctcttcaacatccaagttaaagaggtcacgcagaacgatggcacgacgactattgccaataaccttaccagcacggtccaggtcttcacggactcagactatcagctcccgtacgtgctcgggtcggcgcaccaaggctgtctcccgccgtttccagcggacgtcttcatggtcccacagtatggatacctcaccctgaacaacgggagtcaggcagtaggacgctcttcattttactgcctggagtactttccttcgcagatgctgagaaccggcaacaacttccagtttacttacaccttcgaggacgtgcctttccacagcagctacgcccacagccagagcttggaccggctcatgaacccactcatcgaccagtacctgtactacctgtctcggactcagtccacgggaggtaccgcaggaactcagcagttgctattttctcaggccgggcctaataacatgtcggctcaggccaaaaactggctacccgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagacagagacgtgtacctgcagggtcccatttgggccaaaattcctcacacagatggacactttcacccgtctcctctcatgggcggctttggacttaagcacccgcctcctcagatcctcatcaaaaacacgcctgttcctgcgaatcctccggcagagttttcggctacaaagtttgcttcattcatcactcagtactccacgggacaggtcagcgtggagatcgagtgggagctgcagaaggaaaacagcaaacgctggaatcccgaaattcagtacacttccaactacaacaagtctgttaatgtggactttactgtagacactaatggtgtttatagtgaaccccgccccattggcacccgttaccttacccgtcccctgtaa

SEQ ID NO: 56 - DNA, 2211 bp, Artificial sequence
  Synthetic Sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaagcctacgaccggcagctcgacagcggagacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcattggcaagacaggccagcagcccgctaaaaagagactcaattttggtcagactggcgactcagagtcagttccagacccccagcctctcggagaaccaccagcagcccccacaagtttgggatctaatacaatggcttcaggcggtggcgcaccaatggcagacaataacgagggtgccgatggagtgggtaattcctcaggaaattggcattgcgattccacatggctgggcgacagagtcatcaccaccagcacccgaacctgggccctgcccacctacaacaaccacctctacaaacaaatttccagccaatcaggagccaacaacgacaaccactactttggctacagcaccccctgggggtattttgactttaacagattccactgccacttttcaccacgtgactggcagcgactcattaacaacaactggggattccgacccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaacaactttgagttcagctaccagtttgaggacgtgcccttccacagcagctacgcgcacagccagagcctggaccggctgatgaatcctctcatcgaccaatacctgtattacctgaacagaactcagaatcagtccggaagtgcccaaaacaaggacttgctgtttagccgtgggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacaaaccctgcgaccggagatgtgcatgctatgggagcattacctggcatggtgtggcaagatagagacgtgtacctgcagggtcccatttgggccaaaattcctcacacggatggacactttcacccgtctccgctgatgggcggctttggcctgaaacatcctccgcctcagatcctgatcaagaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 57 - DNA, 2214 bp, Artificial sequence
  Synthetic Sequence
atggctgccgatggttatcttccagattggctcgaggacactctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcggacgagcagtcttccaggcgaaaaagaggcttcttgaacctcttggtctggttgaggaagcggctaagacggctccgggaaaaaagaggccggtagagcactctcctgtggagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaattggggattccggcccaagagactcaacttcaaactcttcaacatccaagtcaaggaggtcacgacgaatgatggcgtcacgaccatcgctaataaccttaccagcacggttcaagtcttctcggactcggagtaccagcttccgtacgtcctcggctctgcgcaccagggctgcctccctccgttcccggcggacgtgttcatgattccgcagtacggctacctaacgctcaacaacggtagtcaggccgtgggacgctcctccttctactgcctggaatactttccttcgcagatgctgagaacgggcaataactttaccttcagctacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggagaacagcaagcgctggaaccccgaggtccagtacacgtccaactacaacaagtccgtcaacgtggaatttacagtggacaacaacggcgtgtattcggaaccgcgcaccatcggcacccgctaccttactcgtaatctgtaa